(19) United States
(12) Patent Application Publication
(10) Pub. No.: US 2006/0194274 A1
(43) Pub. Date: Aug. 31, 2006

(54) Method for producing ketocarotinoids in genetically modified, non-human organisms

(75) Inventors: Ralf Flachmann, Quedlinburg (DE); Christel Renate Schopfer, Quedlinburg (DE); Karin Herbers, Quedlinburg (DE); Irene Kunze, Gatersleben (DE); Matt Sauer, Quedlinburg (DE); Martin Klebsattel, Quedlinburg (DE); Thomas Luck, Neustadt (DE); Dirk Voeste, Limburgerhof (DE); Angelika-Maria Pfeiffer, Birkenheide (DE)

Correspondence Address: CONNOLLY BOVE LODGE & HUTZ, LLP, P O BOX 2207, WILMINGTON, DE, 19899, US

(73) Assignee: SunGene GmbH, GATERSLEBEN (DE)

(21) Appl. No.: 10/569,022

(22) PCT Filed: Jul. 31, 2004
(86) PCT No.: PCT/EP04/08623

§ 371 Date: Feb. 17, 2006

(30) Foreign Application Priority Data
Aug. 18, 2003 (WO) PCT/EP03/09102
Aug. 18, 2003 (EP) PCTEP0309107
Aug. 18, 2003 (EP) PCTEP0309105
Aug. 18, 2003 (EP) PCTEP0309106
Aug. 18, 2003 (EP) PCTEP0309109
Aug. 18, 2003 (EP) PCTEP0309101
Feb. 17, 2004 (DE) 10 204 007 622.7

(51) Int. Cl. C12P 23/00; C12N 15/82; C12N 5/04; A01H 1/00

(52) U.S. Cl. 435/67; 435/419; 435/468; 800/282

(57) ABSTRACT

The present invention relates to a process for the preparation of ketocarotenoids by culturing genetically modified organisms which in comparison with the wild-type have a modified ketolase activity and a modified β-cyclase activity, to the genetically modified organisms, and to their use as foodstuffs and feedstuffs for the production of ketocarotenoid extracts.

The present invention relates to a process for the preparation of ketocarotenoids by culturing genetically modified organisms, which in comparison with the wild-type have a modified ketolase activity and a modified β-cyclase activity, to the genetically modified organisms, and to their use as foodstuffs and feedstuffs for the production of ketocarotenoid extracts.

Carotenoids are synthesized de novo in bacteria, algae, fungi and plants. Ketocarotenoids, that is carotenoids which comprise at least one keto group, such as, for example, astaxanthin, canthaxanthin, echinenone, 3-hydroxyechinenone, 3′-hydroxy-echinenone, adonirubin and adonixanthin are natural antioxidants and pigments which are produced by some algae and microorganisms as secondary metabolites.

On account of their color-imparting properties, the ketocarotenoids and in particular astaxanthin are used as pigmenting aids in animal nutrition, in particular in trout, salmon, and shrimp farming.

The preparation of astaxanthin is nowadays carried out mainly by means of chemical synthesis processes. Natural ketocarotenoids, such as, for example, natural astaxanthin, are nowadays obtained in small amounts in biotechnological processes by culturing algae, for example Haematococcus pluvialis or by fermentation of microorganisms optimized by genetic engineering, and subsequent isolation.

An economic biotechnological process for the preparation of natural ketocarotenoids is therefore of great importance.

Nucleic acids encoding a ketolase and the corresponding protein sequences have been isolated from various organisms and annotated, such as, for example, nucleic acids encoding a ketolase from Agrobacterium aurantiacum (EP 735 137, Accession NO: D58420), from Alcaligenes sp. PC-1 (EP 735137, Accession NO: D58422), Haematococcus pluvialis Flotow em. Wille and Haematoccus pluvialis, NIES-144 (EP 725137, WO 98/18910 and Lotan et al, FEBS Letters 1995, 364,125-128, Accession NO: X86782 and D45881), Paracoccus marcusii (Accession NO: Y15112), Synechocystis sp. Strain PC6803 (Accession NO: NP_{—}442491), Bradyrhizobium sp. (Accession NO: AF218415) and Nostoc sp. PCC 7120 (Kaneko et al, DNA Res. 2001, 8(5), 205-213; Accession NO: AP003592, BAB74888).

EP 735 137 describes the preparation of xanthophylls in microorganisms, such as, for example, E. coli by insertion of ketolase genes (crtW) from Agrobacterium aurantiacum or Alcaligenes sp. PC-1 into microorganisms.

It is known from EP 725 137, WO 98/18910, Kajiwara et al. (Plant Mol. Biol. 1995, 29, 343-352) and Hirschberg et al. (FEBS Letters 1995, 364,125-128) to prepare astaxanthin by insertion of ketolase genes from Haematococcus pluvialis (crtW, crtO or bkt) into E. coli.

Hirschberg et al. (FEBS Letters 1997, 404,129-134) describe the preparation of astaxanthin in Synechococcus by insertion of ketolase genes (crtO) from Haematococcus pluvialis. Sandmann et al. (Photochemistry and Photobiology 2001, 73(5), 551-55) describe an analogous process which, however, leads to the preparation of canthaxanthin and yields only traces of astaxanthin.

WO 98/18910 and Hirschberg et al. (Nature Biotechnology 2000, 18(8), 888-892) describe the synthesis of ketocarotenoids in nectaries of tobacco flowers by insertion of the ketolase gene from Haematococcus pluvialis (crtO) in tobacco.

WO 01/20011 describes a DNA construct for the production of ketocarotenoids, in particular astaxanthin, in seeds of oilseed plants such as rapeseed, sunflower, soybeans and hemp using a seed-specific promoter and a ketolase from Haematococcus pluvialis.

All processes for the preparation of ketocarotenoids described in the prior art and in particular the processes described for the preparation of astaxanthin have the disadvantage that on the one hand the yield is still not satisfactory and on the other hand the transgenic organisms yield a large amount of hydroxylated byproducts, such as, for example, zeaxanthin and adonixanthin.

The invention is therefore based on the object of making available a process for the preparation of ketocarotenoids by culturing genetically modified, nonhuman organisms, or further making available genetically modified, nonhuman organisms which produce ketocarotenoids, which to a lesser extent or no longer have the disadvantages of the prior art described above or produce the desired ketocarotenoids in higher yields.

Accordingly, a process for the preparation of ketocarotenoids by culturing genetically modified, nonhuman organisms which in comparison with the wild-type have a modified ketolase activity and a modified β-cyclase activity has been found, and the modified β-cyclase activity is caused by a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

A “ketolase activity modified in comparison with the wild-type” is understood as meaning, for the case in which the starting organism or wild-type has no ketolase activity, preferably a “ketolase activity caused in comparison with the wild-type”.

A “ketolase activity modified in comparison with the wild-type” is understood as meaning, for the case in which the starting organism or wild-type has a ketolase activity, preferably a “ketolase activity increased in comparison with the wild-type”.

A “β-cyclase activity modified in comparison with the wild-type” is understood as meaning for the case in which the starting organism or wild-type has no β-cyclase activity, preferably a “β-cyclase activity caused in comparison with the wild-type”.

A “β-cyclase activity modified in comparison with the wild-type” is understood as meaning for the case in which the starting organism or wild-type has a β-cyclase activity, preferably a “β-cyclase activity increased in comparison with the wild-type”.

The nonhuman organisms according to the invention such as, for example, microorganisms or plants are preferably, as starting organisms, naturally in the position to produce carotenoids such as, for example, β-carotene or zeaxanthin, or can be placed by genetic modification, such as, for example, reregulation of metabolic pathways or complementation, in the position to produce carotenoids such as, for example, β-carotene or zeaxanthin.

Some organisms are, as starting or wild-type organisms, already in the position to produce ketocarotenoids such as, for example, astaxanthin or canthaxanthin. These organisms, such as, for example, Haematococcus pluvialis, Paracoccus marcusii, Xanthophyllomyces dendrorhous, Bacillus circulans, Chlorococcum, Phaffia rhodozyma, pheasant's-eye, Neochloris wimmeri, Protosiphon botryoides, Scotiellopsis oocystiformis, Scenedesmus vacuolatus, Chlorela zofingiensis, Ankistrodesmus braunii, Euglena sanguinea and Bacillus atrophaeus already have, as a starting or wild-type organism, a ketolase activity and a β-cyclase activity.

The term “wild-type” is understood according to the invention as meaning the corresponding starting organism.

Depending on the context, the term “organism” can be understood as meaning the nonhuman starting organism (wild-type) or a genetically modified, nonhuman organism according to the invention or both.

Preferably and in particular in cases in which the plant or the wild-type cannot be clearly assigned, “wild-type” is in each case understood as meaning a reference organism for the increasing or causing of the ketolase activity, for the increasing or causing of the hydroxylase activity described below, for the increasing or causing of the β-cyclase activity described below, for the increasing of the HMG-CoA reductase activity described below, for the increasing of the (E)-4-hydroxy-3-methylbut-2-enyl diphosphate reductase activity described below, for the increasing of the 1-deoxy-D-xylose 5-phosphate synthase activity described below, for the increasing of the 1-deoxy-D-xylose 5-phosphate reductoisomerase activity described below, for the increasing of the isopentenyl diphosphate Δ-isomerase activity described below, for the increasing of the geranyl diphosphate synthase activity described below, for the increasing of the farnesyl diphosphate synthase activity described below, for the increasing of the geranylgeranyl diphosphate synthase activity described below, for the increasing of the phytoene synthase activity described below, for the increasing of the phytoene desaturase activity described below, for the increasing of the zeta-carotene desaturase activity described below, for the increasing of the crtISO activity described below, for the increasing of the FtsZ activity described below, for the increasing of the MinD activity described below, for the reduction of the ε-cyclase activity described below and for the reduction of the endogenous β-hydroxylase activity described below and the increasing of the content of ketocarotenoids.

This reference organism is for microorganisms which already, as the wild-type, have a ketolase activity, preferably Haematococcus pluvialis.

This reference organism is for microorganisms which already, as the wild-type, have no ketolase activity, preferably Blakeslea.

This reference organism is for plants which already, as the wild-type, have a ketolase activity, preferably Adonis aestivalis, Adonis flammeus or Adonis annuus, particularly preferably Adonis aestivalis.

This reference organism is for plants which already, as the wild-type, have no ketolase activity in petals, preferably Tagetes erecta, Tagetes patula, Tagetes lucida, Tagetes pringlei, Tagetes palmeri, Tagetes minuta or Tagetes campanulata, particularly preferably Tagetes erecta.

Ketolase activity is understood as meaning the enzyme activity of a ketolase.

A ketolase is understood as meaning a protein which has the enzymatic activity to introduce a keto group on the optionally substituted, β-ionone ring of carotenoids.

In particular, a ketolase is understood as meaning a protein which has the enzymatic activity to convert β-carotene to canthaxanthin.

Accordingly, ketolase activity is understood as meaning the amount of β-carotene reacted in a certain time by the protein ketolase or amount of canthaxanthin formed.

In one embodiment of the process according to the invention, the starting organisms used are nonhuman organisms which already, as a wild-type or starting organism, have a ketolase activity, such as, for example, Haematococcus pluvialis, Paracoccus marcusii, Xanthophyllomyces dendrorhous, Bacillus circulans, Chlorococcum, Phaffia rhodozyma, pheasant's eye, Neochloris wimmeri, Protosiphon botryoides, Scotiellopsis oocystiformis, Scenedesmus vacuolatus, Chlorela zofingiensis, Ankistrodesmus braunii, Euglena sanguinea or Bacillus atrophaeus. In this embodiment, the genetic modification causes an increasing of the ketolase activity in comparison with the wild-type or starting organism.

With an increased ketolase activity compared to the wild-type, the amount of β-carotene reacted or the amount of canthaxanthin formed by the protein ketolase in a certain time is increased in comparison with the wild-type.

Preferably, this increasing of the ketolase activity is at least 5%, furthermore preferably at least 20%, furthermore preferably at least 50%, furthermore preferably at least 100%, more preferably at least 300%, even more preferably at least 500%, in particular at least 600% of the ketolase activity of the wild-type.

The determination of the ketolase activity in genetically modified organisms according to the invention and in wild-type or reference organisms is preferably carried out under the following conditions:

The determination of the ketolase activity in plant or microorganism material is carried out following the method of Fraser et al., (J. Biol. Chem. 272(10): 6128-6135, 1997). The ketolase activity in plant or microorganism extracts is determined using the substrates β-carotene and canthaxanthin in the presence of lipid (soybean lecithin) and detergent (sodium cholate). Substrate/product ratios from the ketolase assays are determined by means of HPLC.

The increasing of the ketolase activity can be carried out by various routes, for example by switching off inhibitory regulation mechanisms at the translation and protein level or by increasing the gene expression of a nucleic acid encoding a ketolase compared to the wild-type, for example by induction of the ketolase gene by means of activators or by insertion of nucleic acids encoding a ketolase into the organism.

Increasing the gene expression of a nucleic acid encoding a ketolase is understood according to the invention in this embodiment as also meaning the manipulation of the expression of the organism's own endogenous ketolases. This can be achieved, for example, by modifying the promoter DNA sequence for ketolase-encoding genes. Such a modification, which results in a modified or preferably increased expression rate, at least of an endogenous ketolase gene, can be carried out by deletion or insertion of DNA sequences.

It is possible as described above to modify the expression of at least one endogenous ketolase by the application of exogenous stimuli. This can be carried out by means of special physiological conditions, that is by the application of foreign substances.

In addition, increased expression of at least one endogenous ketolase gene can be achieved by a regulator protein which does not occur or is modified in the wild-type organism interacting with the promoter of these genes.

Such a regulator can be a chimeric protein which consists of a DNA binding domain and a transcription activator domain, such as described, for example, in WO 96/06166.

In a preferred embodiment, the increasing of the ketolase activity compared to the wild-type is carried out by the increasing of the gene expression of a nucleic acid encoding a ketolase.

In a furthermore preferred embodiment, the increasing of the gene expression of a nucleic acid encoding a ketolase is carried out by introduction of nucleic acids which encode ketolases into the organism.

In the transgenic organisms according to the invention, at least one further ketolase gene is therefore present in this embodiment compared to the wild-type. In this embodiment, the genetically modified organism according to the invention preferably has at least one exogenous (=heterologous) nucleic acid, encoding a ketolase, or at least two endogenous nucleic acids encoding a ketolase.

In another preferred embodiment of the process according to the invention, the starting organisms used are nonhuman organisms which, as the wild-type, have no ketolase activity, such as, for example, Blakeslea, Marigold, Tagetes erecta, Tagetes lucida, Tagetes minuta, Tagetes pringlei, Tagetes palmeri and Tagetes campanulata.

In this preferred embodiment, the genetic modification causes the ketolase activity in the organisms. The genetically modified organism according to the invention in this preferred embodiment, in comparison with the genetically unmodified wild-type, thus has a ketolase activity and is thus preferably in the position to express a ketolase transgenically.

In this preferred embodiment, the causing of the gene expression of a nucleic acid encoding a ketolase analogously to the increasing of the gene expression of a nucleic acid encoding a ketolase described above is preferably carried out by insertion of nucleic acids which encode ketolases in the starting organism.

To this end, in both embodiments in principle each ketolase gene, that is each nucleic acid which encodes a ketolase, can be used.

All nucleic acids mentioned in the description can be, for example, an RNA, DNA or cDNA sequence.

In genomic ketolase sequences from eukaryotic sources which comprise introns, in the case in which the host organism is not in the position or cannot be placed in the position to express the corresponding ketolase, preferably already processed nucleic acid sequences, such as the corresponding cDNAs, are to be used.

Examples of nucleic acids encoding a ketolase and the corresponding ketolases which can be used in the process according to the invention are, for example, sequences from

Haematoccus pluvialis, in particular from Haematoccus pluvialis Flotow em. Wille (Accession NO: X86782; nucleic acid: SEQ ID NO: 3, protein SEQ ID NO: 4),

Haematoccus pluvialis, NIES-144 (Accession NO: D45881; nucleic acid: SEQ ID NO: 35, protein SEQ ID NO: 36),

Agrobacterium aurantiacum (Accession NO: D58420; nucleic acid: SEQ ID NO: 37, protein SEQ ID NO: 38),

Alicaligenes spec. (Accession NO: D58422; nucleic acid: SEQ ID NO: 39, protein SEQ ID NO: 40),

Paracoccus marcusii (Accession NO: Y15112; nucleic acid: SEQ ID NO: 41, protein SEQ ID NO: 42).

Synechocystis sp. Strain PC6803 (Accession NO: NP442491; nucleic acid: SEQ ID NO: 43, protein SEQ ID NO: 44).

Bradyrhizobium sp. (Accession NO: AF218415; nucleic acid: SEQ ID NO: 45, protein SEQ ID NO: 46).

Nostoc sp. Strain PCC7120 (Accession NO: AP003592, BAB74888; nucleic acid: SEQ ID NO: 47, protein SEQ ID NO: 48).

Haematococcus pluvialis

(Accession NO: AF534876, MN03484; nucleic acid: SEQ ID NO: 49, protein: SEQ ID NO: 50)

Paracoccus sp. MBIC1143

(Accession NO: D58420, P54972; nucleic acid: SEQ ID NO: 51, protein: SEQ ID NO: 52)

Brevundimonas aurantiaca

(Accession NO: AY166610, MN86030; nucleic acid: SEQ ID NO: 53, protein: SEQ ID NO: 54)

Nodularia spumigena NSOR10

(Accession NO: AY210783, M064399; nucleic acid: SEQ ID NO: 55, protein: SEQ ID NO: 56)

Nostoc punctiforme ATCC 29133

(Accession NO: NZ_AABC01000195, ZP_{—}00111258; nucleic acid: SEQ ID NO: 57, protein: SEQ ID NO: 58)

Nostoc punctiforme ATCC 29133

(Accession NO: NZ_AABC01000196; nucleic acid: SEQ ID NO: 59, protein: SEQ ID NO: 60)

Deinococcus radiodurans R1

(Accession NO: E75561, AE001872; nucleic acid: SEQ ID NO: 61, protein: SEQ ID NO: 62),

Synechococcus sp. WH 8102,

nucleic acid: Acc.-No. NZ_AABD01000001, base pair 1,354,725-1,355,528 (SEQ ID NO: 75), protein: Acc.-No. ZP_{—}00115639 (SEQ ID NO: 76) (annotated as a putative protein),

or sequences derived from these sequences, such as, for example,

the ketolases of the sequence SEQ ID NO: 64 or 66 and the corresponding coding nucleic acid sequences SEQ ID NO: 63 or SEQ ID NO: 65, which arise, for example, by variation/mutation of the sequence SEQ ID NO: 58 or SEQ ID NO: 57,

the ketolases of the sequence SEQ ID NO: 68 or 70 and the corresponding coding nucleic acid sequences SEQ ID NO: 67 or SEQ ID NO: 69, which arise, for example, by variation/mutation of the sequence SEQ ID NO: 60 or SEQ ID NO: 59, or

the ketolases of the sequence SEQ ID NO: 72 or 74 and the corresponding coding nucleic acid sequences SEQ ID NO: 71 or SEQ ID NO: 73, which arise, for example, by variation or mutation of the sequence SEQ ID NO: 76 or SEQ ID NO: 75.

Further natural examples of ketolases and ketolase genes which can be used in the process according to the invention can be easily found, for example, from various organisms whose genomic sequence is known, by means of identity comparisons of the amino acid sequences or of the corresponding back-translated nucleic acid sequences from databases comprising the sequences described above and in particular having the sequences SEQ ID NO: 4 and/or 48 and/or 58 and/or 60.

Further natural examples of ketolases and ketolase genes can furthermore be easily found starting from the nucleic acid sequences described above, in particular starting from the sequences SEQ ID NO: 3 and/or 47 and/or 57 and/or 59 from various organisms whose genomic sequence is not known, by hybridization techniques in a manner known per se.

The hybridization can be carried out under moderate (low stringency) or preferably under stringent (high stringency) conditions.

Such hybridization conditions, which apply for all nucleic acids of the description, are described, for example, in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

For example, the conditions during the washing step can be selected from the range of conditions restricted by those with low stringency (with 2×SSC at 50° C.) and those with high stringency (with 0.2×SSC at 50° C., preferably at 65° C.) (20×SSC: 0.3 M sodium citrate, 3 M sodium chloride, pH 7.0).

Moreover, the temperature during the washing step can be raised from moderate conditions at room temperature, 22° C., up to stringent conditions at 65° C.

The two parameters, salt concentration and temperature, can simultaneously be varied, one of the two parameters can also be kept constant and only the other varied. During the hybridization, denaturing agents such as, for example, formamide or SDS can also be employed. In the presence of 50% formamide, the hybridization is preferably carried out at 42° C.

Some exemplary conditions for hybridization and the washing step are given as a result of:

(1) hybridization conditions with, for example,

(i) 4×SSC at 65° C., or

(ii) 6×SSC at 45° C., or

(iii) 6×SSC at 68° C., 100 mg/ml of denatured fish sperm DNA, or

(iv) 6×SSC, 0.5% SDS, 100 mg/ml of denatured, fragmented salmon sperm DNA at 68° C., or

(v) 6×SSC, 0.5% SDS, 100 mg/ml of denatured, fragmented salmon sperm DNA, 50% formamide at 42° C., or

(vi) 50% formamide, 4×SSC at 42° C., or

(vii) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C., or

(viii) 2× or 4×SSC at 50° C. (moderate conditions), or

(ix) 30 to 40% formamide, 2× or 4×SSC at 42_(moderate conditions).

(2) washing steps for in each case 10 minutes with, for example,

(i) 0.015 M NaCl/0.0015 M sodium citrate/0.1% SDS at 50° C., or

(ii) 0.1×SSC at 65° C., or

(iii) 0.1×SSC, 0.5% SDS at 68° C., or

(iv) 0.1×SSC, 0.5% SDS, 50% formamide at 42° C., or

(v) 0.2×SSC, 0.1% SDS at 42° C., or

(vi) 2×SSC at 65° C. (moderate conditions).

In a preferred embodiment of the process according to the invention, nucleic acids are introduced which encode a protein comprising the amino acid sequence SEQ ID NO: 4 or a sequence derived from this sequence by substitution, insertion or deletion of amino acids, which has an identity of at least 70%, preferably at least 80%, more preferably at least 85%, more preferably at least 90%, more preferably at least 95%, more preferably at least 97%, more preferably at least 98%, particularly preferably at least 99% at the amino acid level with the sequence SEQ ID NO: 4 and the enzymatic properties of a ketolase.

At the same time, it can be a natural ketolase sequence, which can be found as described above, by identity comparison of the sequences from other organisms, or a synthetic ketolase sequence which, starting from the sequence SEQ ID NO: 4, has been modified by synthetic variation, for example by substitution, insertion or deletion of amino acids.

In a further preferred embodiment of the process according to the invention, nucleic acids are employed which encode a protein comprising the amino acid sequence SEQ ID NO: 48 or a sequence derived from this sequence by substitution, insertion or deletion of amino acids, which has an identity of at least 70%, preferably at least 80%, more preferably at least 85%, more preferably at least 90%, more preferably at least 95%, more preferably at least 97%, more preferably at least 98%, particularly preferably at least 99% at the amino acid level with the sequence SEQ ID NO: 48 and the enzymatic properties of a ketolase.

At the same time, it can be a natural ketolase sequence which, as described above, can be found by identity comparison of the sequences from other organisms, or a synthetic ketolase sequence which, starting from the sequence SEQ ID NO: 48, has been modified by synthetic variation, for example by substitution, insertion or deletion of amino acids.

In a further preferred embodiment of the process according to the invention, nucleic acids are introduced which encode a protein comprising the amino acid sequence SEQ ID NO: 58 or a sequence derived from this sequence by substitution, insertion or deletion of amino acids, which has an identity of at least 70%, preferably at least 80%, more preferably at least 85%, more preferably at least 90%, more preferably at least 95%, more preferably at least 97%, more preferably at least 98%, particularly preferably at least 99% at the amino acid level with the sequence SEQ ID NO: 58 and the enzymatic properties of a ketolase.

At the same time, it can be a natural ketolase sequence which, as described above, can be found by identity comparison of the sequences from other organisms, or a synthetic ketolase sequence which, starting from the sequence SEQ ID NO: 58, has been modified by synthetic variation, for example by substitution, insertion or deletion of amino acids.

In a further preferred embodiment of the process according to the invention, nucleic acids are introduced which encode a protein comprising the amino acid sequence SEQ ID NO: 60 or a sequence derived from this sequence by substitution, insertion or deletion of amino acids, which has an identity of at least 70%, preferably at least 80%, more preferably at least 85%, more preferably at least 90%, more preferably at least 95%, more preferably at least 97%, more preferably at least 98%, particularly preferably at least 99% at the amino acid level with the sequence SEQ ID NO: 60 and the enzymatic properties of a ketolase.

At the same time, it can be a natural ketolase sequence which, as described above, can be found by identity comparison of the sequences from other organisms, or a synthetic ketolase sequence which, starting from the sequence SEQ ID NO: 60, has been modified by synthetic variation, for example by substitution, insertion or deletion of amino acids.

The term “substitution” is to be understood in the description for all proteins as meaning the replacement of one or more amino acids by one or more amino acids. Preferably, “conservative replacements” are carried out, in which the replaced amino acid has similar properties to that of the original amino acid, for example replacement of Glu by Asp, Gln by Asn, Val by Ile, Leu by Ile, Ser by Thr.

Deletion is the replacement of an amino acid by a direct bond. Preferred positions for deletions are the termini of the polypeptide and the linkages between the individual protein domains.

Insertions are insertions of amino acids into the polypeptide chain, a direct bond formally being replaced by one or more amino acids.

Identity between two proteins is understood as meaning the identity of the amino acids over the total protein length in each case, in particular the identity which is calculated by comparison with the aid of the Vector NTI Suite 7.1 Software of informax (USA) using the Clustal method (Higgins D G, Sharp P M. Fast and sensitive multiple sequence alignments on a microcomputer. Comput Appl. Biosci. 1989 April; 5(2):151-1) with setting of the following parameters:

Multiple Alignment Parameter:

Gap opening penalty 10

Gap extension penalty 10

Gap separation penalty range 8

Gap separation penalty off

% identity for alignment delay 40

Residue specific gaps off

Hydrophilic residue gap off

Transition weighing 0

Pairwise Alignment Parameter:

FAST algorithm on

K-tuple size 1

Gap penalty 3

Window size 5

Number of best diagonals 5

A protein which has an identity of at least 70% at the amino acid level is accordingly understood as meaning a protein which, on a comparison of its sequence with the determined sequence, in particular has an identity of at least 70% with the above parameter set according to the above program logarithm.

A protein which has, for example, an identity of at least 70% at the amino acid level with the sequence SEQ ID NO: 4 or 48 or 58 or 60, is accordingly understood as meaning a protein, which in a comparison of its sequence with the the sequence SEQ ID NO: 4 or 48 or 58 or 60, in particular according to the above program logarithm has an identity of at least 70% the above parameter set.

Suitable nucleic acid sequences are obtainable, for example, by back-translation of the polypeptide sequence according to the genetic code.

Preferably, those codons are used for this which, according to the organism-specific codon usage, are often used. The codon usage can be easily determined with the aid of computer analyses of other, known genes of the organisms concerned.

In a particularly preferred embodiment, a nucleic acid comprising the sequence SEQ ID NO: 3 is inserted into the plant.

In a further, particularly preferred embodiment, a nucleic acid comprising the sequence SEQ ID NO: 48 is inserted into the plant.

In a further, particularly preferred embodiment, a nucleic acid comprising the sequence SEQ ID NO: 58 is inserted into the plant.

In further, particularly preferred embodiment, a nucleic acid comprising the sequence SEQ ID NO: 60 is inserted into the plant.

All abovementioned ketolase genes can furthermore be prepared in a manner known per se by chemical synthesis from the nucleotide structural units, such as, for example, by fragment condensation of individual overlapping, complementary nucleic acid structural units of the double helix. The chemical synthesis of oligonucleotides can be carried out, for example, in a manner known per se according to the phosphoamidite method (Voet, Voet, 2nd edition, Wiley Press New York, pp. 896-897). The addition of synthetic oligonucleotides and filling of gaps with the aid of the Klenow fragment of the DNA polymerase and ligation reactions and general cloning processes are described in Sambrook et al. (1989), Molecular cloning: A laboratory manual, Cold Spring Harbor Laboratory Press.

As mentioned above, the nonhuman organisms used in the process according to the invention have a modified ketolase activity and a modified β-cyclase activity in comparison with the wild-type, the modified β-cyclase activity being caused by a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

In one embodiment of the process according to the invention, the starting organisms used are nonhuman organisms which already as the wild-type or starting organism have a β-cyclase activity. In this embodiment, the genetic modification brings about an increasing of the β-cyclase activity in comparison with the wild-type or starting organism, the increased β-cyclase activity being caused by a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

β-Cyclase activity is understood as meaning the enzyme activity of a β-cyclase.

A β-cyclase is understood as meaning a protein which has the enzymatic activity to convert a terminal, linear residue of lycopene into a β-ionone ring.

In particular, a β-cyclase is understood as meaning a protein which has the enzymatic activity to convert γ-carotene into β-carotene.

Accordingly, β-cyclase activity is understood as meaning the amount of γ-carotene reacted or amount of β-carotene formed in a certain time by the protein β-cyclase.

With an increased β-cyclase activity compared to the wild-type, in comparison with the wild-type the amount of lycopene or γ-carotene reacted in a certain time by the protein β-cyclase or the amount of γ-carotene formed from lycopene or the amount of β-carotene formed from γ-carotene is thus increased.

Preferably, this increasing of the β-cyclase activity amounts to at least 5%, furthermore preferably at least 20%, furthermore preferably at least 50%, furthermore preferably at least 100%, more preferably at least 300%, even more preferably at least 500%, in particular at least 600% of the β-cyclase activity of the wild-type.

The determination of the β-cyclase activity in genetically modified organisms according to the invention and in wild-type or reference organisms is preferably carried out under the following conditions:

The activity of the β-cyclase is determined in vitro according to Fraser and Sandmann (Biochem. Biophys. Res. Comm. 185(1) (1992) 9-15). Potassium phosphate as a buffer (pH 7.6), lycopene as a substrate, stroma protein from paprika, NADP+, NADPH and ATP are added to a specific amount of organism extract.

Particularly preferably, the determination of the β-cyclase activity is carried out under the following conditions according to Bouvier, d'Harlingue and Camara (Molecular Analysis of carotenoid cyclae inhibition; Arch. Biochem. Biophys. 346(1) (1997) 53-64):

The in-vitro assay is carried out in a volume of 250 μl volume. The batch comprises 50 mM potassium phosphate (pH 7.6), differing amounts of organism extract, 20 nM lycopene, 250 μg of chromoplastidic stroma protein from paprika, 0.2 mM NADP+, 0.2 mM NADPH and 1 mM ATP. NADP/NADPH and ATP are dissolved in 10 ml of ethanol with 1 mg of Tween 80 immediately before addition to the incubation medium. After a reaction time of 60 minutes at 30° C., the reaction is ended by addition of chloroform/methanol (2:1). The reaction products extracted into chloroform are analyzed by means of HPLC.

An alternative assay using radioactive substrate is described in Fraser and Sandmann (Biochem. Biophys. Res. Comm. 185(1) (1992) 9-15).

The increase in the β-cyclase activity can be carried out in various ways, for example by switching off inhibitory regulation mechanisms at the expression and protein level or by increasing the gene expression compared to the wild-type of nucleic acids, encoding a β-cyclase, comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

The increase in the gene expression of the nucleic acids encoding a β-cyclase, compared to the wild-type can likewise be carried out in various ways, for example by induction of the β-cyclase gene by activators or by insertion of one or more β-cyclase-gene copies, that is by inserting at least one nucleic acid encoding a α-cyclase, comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2, in the organism.

Increasing the gene expression of a nucleic acid encoding a β-cyclase, is understood according to the invention also as meaning the manipulation of the expression of the organism's own endogenous β-cyclase, comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

This can be achieved, for example, by modification of the promoter DNA sequence for β-cyclase-encoding genes. Such a modification, which results in an increased expression rate of the gene, can be carried out, for example, by deletion or insertion of DNA sequences.

It is possible, as described above, to modify the expression of the endogenous β-cyclase by the application of exogenous stimuli. This can be carried out by means of particular physiological conditions, that is by the administration of foreign substances.

In addition, a modified or increased expression of an endogenous β-cyclase gene can be achieved by a regulator protein not occurring in the untransformed organism interacting with the promoter of this gene.

Such a regulator can be a chimeric protein which consists of a DNA binding domain and a transcription activator domain, such as described, for example, in WO 96/06166.

In a preferred embodiment, the increase in the gene expression of a nucleic acid encoding a β-cyclase is carried out by insertion into the organism of at least one nucleic acid encoding a β-cyclase, comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

In the transgenic organisms according to the invention, at least one further β-cyclase gene is thus present in this embodiment compared to the wild-type. In this embodiment, the genetically modified organism according to the invention preferably has at least one exogenous (=heterologous) nucleic acid encoding a β-cyclase, or at least two endogenous nucleic acids encoding a β-cyclase.

In another, preferred embodiment of the process according to the invention, the starting organisms used are nonhuman organisms, which as the wild-type have no β-cyclase activity. In this, less preferred embodiment, the genetic modification causes the β-cyclase activity in the organisms. The genetically modified organism according to the invention thus has in this embodiment in comparison with the genetically unmodified wild-type a β-cyclase activity and is thus preferably able to express transgenically a β-cyclase.

In this preferred embodiment, the causing of the gene expression of a nucleic acid encoding a β-cyclase analogously to the increasing described above of the gene expression of a nucleic acid encoding a β-cyclase is preferably carried out by inserting nucleic acids which encode β-cyclase into the starting organism.

To this end, in both embodiments in principle any β-cyclase gene, that is any nucleic acid, which encodes a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2, can be used.

With genomic β-cyclase nucleic acid sequences from eukaryotic sources, which comprise introns, for the case in which the host organism is not in the position or cannot be put in the position of expressing the corresponding β-cyclase, preferably already processed nucleic acid sequences, such as the corresponding cDNAs, are to be used.

A particularly preferred β-cyclase is the chromoplast-specific β-cyclase from tomato (AAG21133) (nucleic acid: SEQ ID No. 1; protein: SEQ ID No. 2).

The β-cyclase genes which can be used according to the invention are nucleic acids which encode proteins, comprising the amino acid sequence SEQ ID NO: 2 or a sequence derived from this sequence by substitution, insertion or deletion of amino acids, which has an identity of at least 70%, preferably at least 80%, more preferably at least 85%, even more preferably at least 90%, most preferably at least 95% at the amino acid level with the sequence SEQ ID NO: 2, and the enzymatic properties of a β-cyclase.

Further examples of β-cyclases and β-cyclase genes can easily be found, for example, from various organisms whose genomic sequence is known, as described above by homology comparisons of the amino acid sequences or of the corresponding back-translated nucleic acid sequences from databases with the SEQ ID NO: 2.

Further examples of β-cyclases and β-cyclase genes can furthermore easily be found in a manner known per se, for example, starting from the sequence SEQ ID NO: 1 of various organisms whose genomic sequence is not known, by hybridization and PCR techniques.

In a further particularly preferred embodiment, for increasing the β-cyclase activity, nucleic acids which encode proteins comprising the amino acid sequence of the β-cyclase of the sequence SEQ ID NO: 2 are introduced into organisms.

Suitable nucleic acid sequences are obtainable, for example, by back-translation of the polypeptide sequence according to the genetic code.

Preferably, for this those codons are used which are often used according to the organism-specific codon usage. The codon usage can easily be determined with the aid of computer analyses of other, known genes of the organisms concerned.

In a particularly preferred embodiment, a nucleic acid comprising the sequence SEQ ID NO: 1 is introduced into the organism.

All abovementioned β-cyclase genes can furthermore be prepared in a manner known per se by chemical synthesis from the nucleotide structural units, such as, for example, by fragment condensation of individual overlapping, complementary nucleic acid structural units of the double helix. The chemical synthesis of oligonucleotides can be carried out, for example, in a known manner, according to the phosphoamidite method (Voet, Voet, 2nd edition, Wiley Press New York, pages 896-897). The addition of synthetic oligonucleotides and filling of gaps with the aid of the Klenow fragment of the DNA-Polymerase and ligation reactions and general cloning processes are described in Sambrook et al. (1989), Molecular cloning: A laboratory manual, Cold Spring Harbor Laboratory Press.

In a preferred embodiment, nonhuman organisms are cultured which, compared to the wild-type, in addition to the modified ketolase activity and modified β-cyclase activity have a modified hydroxylase activity.

A “modified hydroxylase activity in comparison with the wild-type” is understood for the case in which the starting organism or wild-type has no hydroxylase activity as preferably meaning a “caused hydroxylase activity in comparison with the wild-type”.

A “modified hydroxylase activity in comparison with the wild-type” is understood for the case in which the starting organism or wild-type has a hydroxylase activity as preferably meaning a “increased hydroxylase activity in comparison with the wild-type”.

Accordingly, in a preferred embodiment nonhuman organisms are cultured which, compared to the wild-type, in addition to the modified ketolase activity and modified β-cyclase activity have a caused or increased hydroxylase activity.

Hydroxylase activity is understood as meaning the enzyme activity of a hydroxylase.

A hydroxylase is understood as meaning a protein which has the enzymatic activity to introduce a hydroxyl group on the optionally substituted β-ionone ring of carotenoids.

In particular, a hydroxylase is understood as meaning a protein which has the enzymatic activity to convert β-carotene to zeaxanthin or canthaxanthin to astaxanthin.

Accordingly, hydroxylase activity is understood as meaning the amount of β-carotene or canthaxanthin reacted in a certain time by the protein hydroxylase or amount of zeaxanthin or astaxanthin formed.

With an increased hydroxylase activity compared to the wild-type, in comparison with the wild-type the amount of β-carotene or cantaxantin reacted or the amount of zeaxanthin or astaxanthin formed in a certain time by the protein hydroxylase is thus increased.

Preferably, this increase in the hydroxylase activity amounts to at least 5%, furthermore preferably at least 20%, furthermore preferably at least 50%, furthermore preferably at least 100%, more preferably at least 300%, even more preferably at least 500%, in particular at least 600% of the hydroxylase activity of the wild-type.

The determination of the hydroxylase activity in organism genetically modified according to the invention and in wild-type or reference organisms is preferably carried out under the following conditions:

The activity of the hydroxylase is determined in vitro according to Bouvier et al. (Biochim. Biophys. Acta 1391 (1998), 320-328). Ferredoxin, ferredoxin-NADP oxidoreductase, catalase, NADPH and beta-carotene with mono- and digalactosylglycerides are added to a specific amount of organism extract.

Particularly preferably, the determination of the hydroxylase activity is carried out under the following conditions according to Bouvier, Keller, d'Harlingue and Camara (Xanthophyll biosynthesis: molecular and functional characterization of carotenoid hydroxylases from pepper fruits (Capsicum annuum L.; Biochim. Biophys. Acta 1391 (1998), 320-328):

The in-vitro assay is carried out in a volume of 0.250 ml volume. The batch comprises 50 mM potassium phosphate (pH 7.6), 0.025 mg ferredoxin from spinach, 0.5 units of ferredoxin-NADP+ oxidoreductase from spinach, 0.25 mM NADPH, 0.010 mg beta-carotene (emulsified in 0.1 mg Tween 80), 0.05 mM of a mixture of mono- and digalactosylglycerides (1:1), 1 unit of catalase, 0.2 mg of bovine serum albumin and organism extract in differing volume. The reaction mixture is incubated for 2 hours at 30° C. The reaction products are extracted using organic solvent such as acetone or chloroform/methanol (2:1) and determined by means of HPLC.

The increase in or causing of the hydroxylase activity can be carried out in various ways, for example by switching off inhibitory regulation mechanisms at the expression and protein level or by increasing or causing the gene expression of nucleic acids encoding a hydroxylase compared to the wild-type.

The increase in or causing of the gene expression of the nucleic acids encoding a hydroxylase compared to the wild-type can likewise be carried out in various ways, for example by induction of the hydroxylase gene, by activators or by insertion of one or more hydroxylase gene copies, that is by insertion of at least one nucleic acid encoding a hydroxylase into the organism.

Increase in the gene expression of a nucleic acid encoding a hydroxylase is understood according to the invention also as meaning the manipulation of the expression of the organism's own, endogenous hydroxylase.

This can be achieved, for example, by modification of the promoter DNA sequence for genes encoding hydroxylases. Such a modification, which results in an increased expression rate of the gene, can be carried out, for example, by deletion or insertion of DNA sequences.

It is possible, as described above, to modify the expression of the endogenous hydroxylase by the application of exogenous stimuli. This can be carried out by particular physiological conditions, that is by the administration of foreign substances.

In addition, a caused or increased expression of an endogenous hydroxylase gene can be achieved by interacting a regulator protein not occurring in the untransformed organism with the promoter of this gene.

Such a regulator can be a chimeric protein which consists of a DNA binding domain and a transcription activator domain, such as described, for example, in WO 96/06166.

In a preferred embodiment, the increase in or causing of the gene expression of a nucleic acid encoding a hydroxylase can be carried out by insertion of at least one nucleic acid encoding a hydroxylase into the organism.

For this, in principle any hydroxylase gene, that is any nucleic acid which encodes a hydroxylase, can be used.

With genomic hydroxylase sequences from eukaryotic sources which comprise introns, in the case in which the host plant is not in the position or cannot be put in the position to express the corresponding hydroxylase, preferably already processed nucleic acid sequences, such as the corresponding cDNAs are to be used.

Examples of a hydroxylase genes are:

a nucleic acid encoding a hydroxylase from Haematococcus pluvialis, Accession AX038729, WO 0061764); (nucleic acid: SEQ ID NO: 77, protein: SEQ ID NO: 78),

and also hydroxylases with the following accession numbers:

|emb|CAB55626.1, CAA70427.1, CAA70888.1, CAB55625.1, AF499108_{—}1, AF315289_{—}1, AF296158_{—}1, AAC49443.1, NP_{—}194300.1, NP_{—}200070.1, AAG10430.1, CAC06712.1, AAM88619.1, CAC95130.1, AAL80006.1, AF162276_{—}1, AAO53295.1, MN85601.1, CRTZ_ERWHE, CRTZ_PANAN, BAB79605.1, CRTZ_ALCSP, CRTZ_AGRAU, CAB56060.1, ZP_{—}00094836.1, AAC44852.1, BAC77670.1, NP_{—}745389.1, NP_{—}344225.1, NP_{—}849490.1, ZP_{—}00087019.1, NP_{—}503072.1, NP_{—}852012.1, NP_{—}115929.1, ZP_{—}00013255.1

A particularly preferred hydroxylase is furthermore the hydroxylase from tomato (Accession Y14810) (nucleic acid: SEQ ID NO: 5; protein: SEQ ID NO. 6).

In the preferred transgenic organisms according to the invention, at least one further hydroxylase gene is thus present in this preferred embodiment compared to the wild-type.

In this preferred embodiment, the genetically modified organism has, for example, at least one exogenous nucleic acid encoding a hydroxylase or at least two endogenous nucleic acids encoding a hydroxylase.

Preferably, in the preferred embodiment described above, the hydroxylase genes used are nucleic acids which encode proteins comprising the amino acid sequence SEQ ID NO: 6 or a sequence derived from this sequence by substitution, insertion or deletion of amino acids, which has an identity of at least 70%, preferably at least 80%, more preferably at least 85%, even more preferably at least 90%, most preferably at least 95% at the amino acid level with the sequence SEQ ID NO: 6, and which have the enzymatic properties of a hydroxylase.

Further examples of hydroxylases and hydroxylase genes can easily be found, for example, from various organisms whose genomic sequence is known, as described above, by homology comparisons of the amino acid sequences or of the corresponding back-translated nucleic acid sequences from databases with the SeQ ID NO: 6.

Further examples of hydroxylases and hydroxylase genes can furthermore be easily found, for example, starting from the sequence SEQ ID NO: 5 of various organisms whose genomic sequence is not known, as described above, by hybridization and PCR techniques in a manner known per se.

In a further particularly preferred embodiment, for increasing the hydroxylase activity nucleic acids which encode proteins comprising the amino acid sequence of the hydroxylase of the sequence SEQ ID NO: 6 are introduced into organisms.

Suitable nucleic acid sequences are obtainable, for example, by back-translation of the polypeptide sequence according to the genetic code.

Preferably, for this those codons are used which are often used according to the organism-specific codon usage. The codon usage can be easily determined with the aid of computer analyses of other, known genes of the organisms concerned.

In a particularly preferred embodiment, a nucleic acid comprising the sequence SEQ ID NO: 5 is introduced into the organism.

All abovementioned hydroxylase genes can furthermore be prepared in a manner known per se by chemical synthesis from the nucleotide structural units, such as, for example, by fragment condensation of individual overlapping, complimentary nucleic acid structural units of the double helix. The chemical synthesis of oligonucleotides can be carried out, for example, in a known manner, according to the phosphoamidite method (Voet, Voet, 2nd edition, Wiley Press New York, pages 896-897). The addition of synthetic oligonucleotides and filling of gaps with the aid of the Klenow fragment of the DNA polymerase and ligation reactions and also general cloning processes are described in Sambrook et al. (1989), Molecular cloning: A laboratory manual, Cold Spring Harbor Laboratory Press.

Particularly preferably, genetically modified nonhuman organisms are employed in the process according to the invention which, as starting organisms, have a β-cyclase activity and no ketolase activity, the genetically modified organisms in comparison with the wild-type having an increased β-cyclase activity, caused by a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2 and have a caused ketolase activity.

Particularly preferably, genetically modified nonhuman organisms are furthermore employed in the process according to the invention which, as starting organisms, have no β-cyclase activity and no ketolase activity, the genetically modified organisms in comparison with the wild-type having a caused β-cyclase activity, caused by a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2 and have a caused ketolase activity.

Particularly preferably, genetically modified nonhuman organisms are furthermore employed in the process according to the invention which, as starting organisms, have a β-cyclase activity and a ketolase activity, the genetically modified organisms in comparison with the wild-type having an increased β-cyclase activity caused by a β-cyclase, comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2 and have an increased ketolase activity.

Particularly preferably, genetically modified nonhuman organisms are employed in the process according to the invention which, as starting organisms, have a β-cyclase activity, no ketolase activity and no hydroxylase activity, the genetically modified organisms in comparison with the wild-type having an increased β-cyclase activity, caused by a β-cyclase, comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2, and have a caused ketolase activity and a caused hydroxylase activity.

Particularly preferably genetically modified nonhuman organisms are employed in the process according to the invention which, as starting organisms, have a β-cyclase activity, a hydroxylase activity and no ketolase activity, the genetically modified organisms in comparison with the wild-type having an increased β-cyclase activity caused by a β-cyclase, comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2, an increased hydroxylase activity and a caused ketolase activity.

Particularly preferably, genetically modified, nonhuman organisms are furthermore employed in the process according to the invention which, as starting organisms, have no β-cyclase activity, no hydroxylase activity and no ketolase activity, the genetically modified organisms in comparison with the wild-type having a caused β-cyclase activity, caused by a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2, and have a caused hydroxylase activity and a caused ketolase activity.

Particularly preferably, genetically modified nonhuman organisms are furthermore employed in the process according to the invention which, as starting organisms, have a β-cyclase activity, a hydroxylase activity and a ketolase activity, the genetically modified organisms in comparison with the wild-type having an increased β-cyclase activity caused by a β-cyclase, comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2, an increased β-cyclase activity, an increased hydroxylase activity and an increased ketolase activity.

In a further preferred embodiment, genetically modified, nonhuman organisms are cultured which additionally compared to the wild-type have an increased activity of at least one of the activities selected from the group consisting of HMG-CoA reductase activity, (E)-4-hydroxy-3-methylbut-2-enyl diphosphate reductase activity, 1-deoxy-D-xylose-5-phosphate synthase activity, 1-deoxy-D-xylose-5-phosphate reductoisomerase activity, isopentenyl diphosphate Δ-isomerase activity, geranyl diphosphate synthase activity, farnesyl diphosphate synthase activity, geranylgeranyl diphosphate synthase activity, phytoene synthase activity, phytoene desaturase activity, zeta-carotene desaturase activity, crtISO activity, FtsZ activity and MinD activity.

HMG-CoA reductase activity is understood as meaning the enzyme activity of an HMG-CoA reductase (3-hydroxy-3-methylglutaryl-coenzyme A reductase).

An HMG-CoA reductase is understood as meaning a protein which has the enzymatic activity to convert 3-hydroxy-3-methyl-glutaryl-coenzyme A to mevalonate.

Accordingly, HMG-CoA reductase activity is understood as meaning the amount of 3-hydroxy-3-methyl-glutaryl-coenzyme A reacted or amount of mevalonate formed in a certain time by the protein HMG-CoA reductase.

With an increased HMG-CoA reductase activity compared to the wild-type, in comparison with the wild-type the amount of 3-hydroxy-3-methyl-glutaryl-coenzyme A reacted or the amount of mevalonate formed in a certain time is thus increased by the protein HMG-CoA reductase.

Preferably, this increase in the HMG-CoA reductase activity amounts to at least 5%, furthermore preferably at least 20%, furthermore preferably at least 50%, furthermore preferably at least 100%, more preferably at least 300%, even more preferably at least 500%, in particular at least 600%, of the HMG-CoA reductase activity of the wild-type.

The determination of the HMG-CoA reductase activity in genetically modified organism according to the invention and in wild-type or reference organisms is preferably carried out under the following conditions:

Frozen organism material is homogenized by intensive grinding in liquid nitrogen in a mortar and pestle and extracted with extraction buffer in a ratio of 1:1 to 1:20. The particular ratio depends on the enzyme activities in the available organism material, such that a determination and quantification of the enzyme activities within the linear measurement range is possible. Typically, the extraction buffer can consist of 50 mM HEPES-KOH (pH 7.4), 10 mM MgCl₂, 10 mM KCl, 1 mM EDTA, 1 mM EGTA, 0.1% (v/v) Triton X-100, 2 mM ε-aminocaproic acid, 10% glycerol, 5 mM KHCO₃. Shortly before the extraction, 2 mM DTT and 0.5 mM PMSF are added.

The activity of the HMG-CoA reductase can be measured according to published descriptions (e.g. Schaller, Grausem, Benveniste, Chye, Tan, Song and Chua, Plant Physiol. 109 (1995), 761-770; Chappell, Wolf, Proulx, Cuellar and Saunders, Plant Physiol. 109 (1995) 1337-1343). Organism tissue can be homogenized and extracted in cold buffer (100 mM potassium phosphate (pH 7.0), 4 mM MgCl₂, 5 mM DTT). The homogenizate is centrifuged for 15 minutes at 10.000 g at 4C. The supernatant is then centrifuged again at 100.000 g for 45-60 minutes. The activity of the HMG-CoA reductase is determined in the supernatant and in the pellet of the microsomal fraction (after resuspending in 100 mM potassium phosphate (pH 7.0) and 50 mM DTT). Aliquots of the solution and of the suspension (the protein content of the suspension corresponds to approximately 1-10 μg) are incubated in 100 mM potassium phosphate buffer (pH 7.0 with 3 mM NADPH and 20 μM (¹⁴C)HMG-CoA (58 μCi/μM) ideally in a volume of 26 μl for 15-60 minutes at 30C. The reaction is terminated by the addition of 5 μl of mevalonolactone (1 mg/ml) and 6 N HCl. After addition, the mixture is incubated at room temperature for 15 minutes. The (¹⁴C)-mevalonate formed in the reaction is quantified by adding 125 μl of a saturated potassium phosphate solution (pH 6.0) and 300 μl of ethyl acetate. The mixture is mixed well and centrifuged. The radioactivity can be determined by means of scintillation measurement.

(E)-4-Hydroxy-3-methylbut-2-enyl diphosphate reductase activity, also designated as lytB or IspH, is understood as meaning the enzyme activity of a (E)-4-hydroxy-3-methylbut-2-enyl diphosphate reductase.

A (E)-4-hydroxy-3-methylbut-2-enyl diphosphate reductase is understood as meaning a protein which has the enzymatic activity to convert (E)-4-hydroxy-3-methylbut-2-enyl diphosphate to isopentenyl diphosphate and dimethylallyl diphosphate.

Accordingly, (E)-4-hydroxy-3-methylbut-2-enyl diphosphate reductase activity is understood as meaning the amount of (E)-4-hydroxy-3-methylbut-2-enyl diphosphate reacted or amount of isopentenyl diphosphate and/or dimethylallyl diphosphate formed in a certain time by the protein (E)-4-hydroxy-3-methylbut-2-enyl diphosphate reductase.

With an increased (E)-4-hydroxy-3-methylbut-2-enyl diphosphate reductase activity compared to the wild-type, the amount of (E)-4-hydroxy-3-methylbut-2-enyl diphosphate reacted or the amount of isopentenyl diphosphate and/or dimethylallyl diphosphate formed in comparison with the wild-type in a certain time is thus increased by the protein (E)-4-hydroxy-3-methylbut-2-enyl diphosphate reductase.

Preferably, this increase in the (E)-4-hydroxy-3-methylbut-2-enyl diphosphate reductase activity amounts to at least 5%, furthermore preferably at least 20%, furthermore preferably at least 50%, furthermore preferably at least 100%, more preferably at least 300%, even more preferably at least 500%, in particular at least 600% of the (E)-4-hydroxy-3-methylbut-2-enyl diphosphate reductase activity of the wild-type.

The determination of the (E)-4-hydroxy-3-methylbut-2-enyl diphosphate reductase activity in genetically modified, nonhuman organisms according to the invention and in wild-type or reference organisms is preferably carried out under the following conditions:

Frozen organism material is homogenized by intensive grinding in liquid nitrogen in a mortar and pestle and extracted with extraction buffer in a ratio of 1:1 to 1:20. The particular ratio depends on the enzyme activities in the available organism material, such that a determination and quantification of the enzyme activities within the linear measurement range is possible. Typically, the extraction buffer can consist of 50 mM HEPES-KOH (pH 7.4), 10 mM MgCl₂, 10 mM KCl, 1 mM EDTA, 1 mM EGTA, 0.1% (v/v) Triton X-100, 2 mM ε-aminocaproic acid, 10% glycerol, 5 mM KHCO₃. Shortly before the extraction, 2 mM DTT and 0.5 mM PMSF are added.

The determination of the (E)-4-Hydroxy-3-methylbut-2-enyl diphosphate reductase activity can be effected by means of immunological detection. The preparation of specific antibodies has been described by Rohdich and colleagues (Rohdich, Hecht, Gärtner, Adam, Krieger, Amslinger, Arigoni, Bacher and Eisenreich: Studies on the nonmevalonate terpene biosynthetic pathway: metabolic role of IspH (LytB) protein, Natl. Acad. Natl. Sci. USA 99 (2002), 1158-1163). For the determination of the catalytic activity, Altincicek and colleagues (Altincicek, Duin, Reichenberg, Hedderich, Kollas, Hintz, Wagner, Wiesner, Beck and Jomaa: LytB protein catalyzes the terminal step of the 2-C-methyl-D-erythritol-4-phosphate pathway of isoprenoid biosynthesis; FEBS Letters 532 (2002,) 437-440) describe an in vitro system which monitors the reduction of (E)-4-hydroxy-3-methyl-but-2-enyl diphosphate to isopentenyl diphosphate and dimethylallyl diphosphate.

1-Deoxy-D-xylose-5-phosphate synthase activity is understood as meaning the enzyme activity of a 1-deoxy-D-xylose-5-phosphate synthase.

A 1-deoxy-D-xylose-5-phosphate synthase is understood as meaning a protein which has the enzymatic activity to convert hydroxyethyl-ThPP and glyceraldehyde 3-phosphate to 1-deoxy-D-xylose 5-phosphate.

Accordingly, 1-deoxy-D-xylose-5-phosphate synthase activity is understood as meaning the amount of hydroxyethyl-ThPP and/or glyceraldehyde 3-phosphate reacted or amount of 1-deoxy-D-xylose-5-phosphate formed in a certain time by the protein 1-deoxy-D-xylose-5-phosphate synthase.

With an increased 1-deoxy-D-xylose-5-phosphate synthase activity compared to the wild-type, the amount of hydroxyethyl-ThPP and/or glyceraldehyde 3-phosphate reacted or the amount of -deoxy-D-xylose-5-phosphate formed is thus increased in a certain time by the protein 1-deoxy-D-xylose-5-phosphate synthase in comparison with the wild-type.

Preferably, this increase in the 1-deoxy-D-xylose-5-phosphate synthase activity amounts to at least 5%, furthermore preferably at least 20%, furthermore preferably at least 50%, furthermore preferably at least 100%, more preferably at least 300%, even more preferably at least 500%, in particular at least 600%, of the 1-deoxy-D-xylose-5-phosphate synthase activity of the wild-type.

The determination of the 1-deoxy-D-xylose-5-phosphate synthase activity in genetically modified organisms according to the invention and in wild-type or reference organisms is preferably carried out under the following conditions:

Frozen organism material is homogenized by intensive grinding in liquid nitrogen in a mortar and pestle and extracted with extraction buffer in a ratio of 1:1 to 1:20. The particular ratio depends on the enzyme activities in the available organism material, such that a determination and quantification of the enzyme activities within the linear measurement range is possible. Typically, the extraction buffer can consist of 50 mM HEPES-KOH (pH 7.4), 10 mM MgCl₂, 10 mM KCl, 1 mM EDTA, 1 mM EGTA, 0.1% (v/v) Triton X-100, 2 mM ε-aminocaproic acid, 10% glycerol, 5 mM KHCO₃. Shortly before the extraction, 2 mM DTT and 0.5 mM PMSF are added.

The reaction solution (50-200 ul) for the determination of the D-1-deoxyxylulose-5-phosphate synthase activity (DXS) consists of 100 mM Tris-HCl (pH 8.0), 3 mM MgCl₂, 3 mM MnCl₂, 3 mM ATP, 1 mM thiamine diphosphate, 0.1% Tween-60, 1 mM potassium fluoride, 30 υM (2-¹⁴C)-pyruvate (0.5 υCi), 0.6 mM DL-glyceraldehyde 3-phosphate. The organism extract is incubated at 37C for 1 to 2 hours in the reaction solution. The reaction is then stopped by heating to 80C for 3 minutes. After centrifugation at 13.000 revolutions/minute for 5 minutes, the supernatant is evaporated, the residue is resuspended in 50 υl of methanol, applied to a TLC plate for thin layer chromatography (Silica-Gel 60, Merck, Darmstadt) and separated in N-propyl alcohol/ethyl acetate/water (6:1:3; v/v/v). In the course of this, radiolabeled D-1-deoxyxylulose-5-phosphate (or D-1-deoxyxylulose) of (2-¹⁴C)-pyruvate separates. Quantification is carried out by means of a scintillation counter. The method was described in Harker and Bramley (FEBS Letters 448 (1999) 115-119). Alternatively, a fluorometric assay for the determination of the DXS synthase activity of Querol and colleagues has been described (Analytical Biochemistry 296 (2001) 101-105).

1-Deoxy-D-xylose-5-phosphate reductoisomerase activity is understood as meaning the enzyme activity of a 1-deoxy-D-xylose-5-phosphate reductoisomerase, also called 1-deoxy-D-xylulose-5-phosphate reductoisomerase.

A 1-deoxy-D-xylose-5-phosphate reductoisomerase is understood as meaning a protein which has the enzymatic activity to convert 1-deoxy-D-xylose-5-phosphate to 2-C-methyl-D-erythritol 4-phosphate.

Accordingly, 1-deoxy-D-xylose-5-phosphate reductoisomerase activity is understood as meaning the amount of 1-deoxy-D-xylose-5-phosphate reacted or amount of 2-C-methyl-D-erythritol 4-phosphate formed in a certain time by the protein 1-deoxy-D-xylose-5-phosphate reductoisomerase.

With an increased 1-deoxy-D-xylose-5-phosphate reductoisomerase activity compared to the wild-type, the amount of 1-deoxy-D-xylose-5-phosphate reacted or the amount of 2-C-methyl-D-erythritol 4-phosphate formed in a certain time is thus increased by the protein 1-deoxy-D-xylose-5-phosphate reductoisomerase in comparison with the wild-type.

Preferably, this increase in the 1-deoxy-D-xylose-5-phosphate reductoisomerase activity amounts to at least 5%, furthermore preferably at least 20%, furthermore preferably at least 50%, furthermore preferably at least 100%, more preferably at least 300%, even more preferably at least 500%, in particular at least 600%, of 1-deoxy-D-xylose-5-phosphate reductoisomerase activity of the wild-type.

The determination of the 1-deoxy-D-xylose-5-phosphate reductoisomerase activity in genetically modified organisms according to the invention and in wild-type or reference organisms is preferably carried out under the following conditions:

Frozen organism material is homogenized by intensive grinding in liquid nitrogen in a mortar and pestle and extracted with extraction buffer in a ratio of 1:1 to 1:20. The particular ratio depends on the enzyme activities in the available organism material, such that a determination and quantification of the enzyme activities within the linear measurement range is possible. Typically, the extraction buffer can consist of 50 mM HEPES-KOH (pH 7.4), 10 mM MgCl₂, 10 mM KCl, 1 mM EDTA, 1 mM EGTA, 0.1% (v/v) Triton X-100, 2 mM ε-aminocaproic acid, 10% glycerol, 5 mM KHCO₃. Shortly before the extraction, 2 mM DTT and 0.5 mM PMSF are added.

The activity of the D-1-deoxyxylulose-5-phosphate reductoisomerase (DXR) is measured in a buffer consisting of 100 mM Tris-HCl (pH 7.5), 1 mM MnCl₂, 0.3 mM NADPH and 0.3 mM 1-deoxy-D-xylulose-4-phosphate, which, for example, can be synthesized enzymatically (Kuzuyama, Takahashi, Watanabe and Seto: Tetrahedon letters 39 (1998) 4509-4512). The reaction is started by addition of the organism extract. The reaction volume can typically amount to 0.2 to 0.5 ml; incubation is carried out at 37C for 30-60 minutes. During this time, the oxidation of NADPH is monitored photometrically at 340 nm.

Isopentenyl diphosphate Δ-isomerase activity is understood as meaning the enzyme activity of an isopentenyl diphosphate Δ-isomerase.

An isopentenyl diphosphate Δ-isomerase is understood as meaning a protein which has the enzymatic activity to convert isopentenyl diphosphate to dimethylallyl phosphate.

Accordingly, isopentenyl diphosphate Δ-isomerase activity is understood as meaning the amount of isopentenyl diphosphate reacted or amount of dimethylallyl phosphate formed in a certain time by the protein isopentenyl diphosphate D-Δ-isomerase.

With an increased isopentenyl diphosphate Δ-isomerase activity compared to the wild-type, in comparison with the wild-type the amount of isopentenyl diphosphate reacted or the amount of dimethylallyl phosphate formed in a certain time is thus increased by the protein isopentenyl diphosphate Δ-isomerase.

Preferably, this increase in the isopentenyl diphosphate Δ-isomerase activity amounts to at least 5%, furthermore preferably at least 20%, furthermore preferably at least 50%, furthermore preferably at least 100%, more preferably at least 300%, even more preferably at least 500%, in particular at least 600%, of the isopentenyl diphosphate Δ-isomerase activity of the wild-type.

The determination of the isopentenyl diphosphate Δ-isomerase activity in genetically modified organisms according to the invention and in wild-type or reference organisms is preferably carried out under the following conditions:

Frozen organism material is homogenized by intensive grinding in liquid nitrogen in a mortar and pestle and extracted with extraction buffer in a ratio of 1:1 to 1:20. The particular ratio depends on the enzyme activities in the available organism material, such that a determination and quantification of the enzyme activities within the linear measurement range is possible. Typically, the extraction buffer can consist of 50 mM HEPES-KOH (pH 7.4), 10 mM MgCl₂, 10 mM KCl, 1 mM EDTA, 1 mM EGTA, 0.1% (v/v) Triton X-100, 2 mM ε-aminocaproic acid, 10% glycerol, 5 mM KHCO₃. Shortly before the extraction, 2 mM DTT and 0.5 mM PMSF are added.

Determinations of activity of the isopentenyl diphosphate isomerase (IPP isomerase) can be carried out according to the method presented by Fraser and colleagues (Fraser, Römer, Shipton, Mills, Kiano, Misawa, Drake, Schuch and Bramley: Evaluation of transgenic tomato plants expressing an additional phytoene synthase in a fruit-specific manner; Proc. Natl. Acad. Sci. USA 99 (2002), 1092-1097, based on Fraser, Pinto, Holloway and Bramley, Plant Journal 24 (2000), 551-558). For enzyme measurements, incubations with 0.5 υCi of (1-¹⁴C)IPP (isopentenyl pyrophosphate) (56 mCi/mmol, Amersham plc) as a substrate are carried out in 0.4 M Tris-HCl (pH 8.0) with 1 mM DTT, 4 mM MgCl₂, 6 mM MnCl₂, 3 mM ATP, 0.1% Tween 60, 1 mM potassium fluoride in a volume of approximately 150-500 υl. Extracts are mixed with buffer (e.g. in the ratio 1:1) and incubated for at least 5 hours at 28° C. Approximately 200 υl of methanol are then added and, by addition of concentrated hydrochloric acid (final concentration 25%) and acid hydrolysis is carried out for approximately 1 hour at 37C. Subsequently, a double extraction is carried out (in each case 500 υl) with petroleum ether (mixed with 10% diethyl ether). The radioactivity in an aliquot of the hyperphase is determined by means of a scintillation counter. The specific enzyme activity can be determined in a short incubation of 5 minutes, since short reaction times suppresses the formation of reaction byproducts (see Lützow and Beyer: The isopentenyl phosphate Δ-isomerase and its relation to the phytoene synthase complex in daffodil chromoplasts; Biochim. Biophys. Acta 959 (1988), 118-126).

Geranyl diphosphate synthase activity is understood as meaning the enzyme activity of a geranyl diphosphate synthase.

A geranyl diphosphate synthase is understood as meaning a protein which has the enzymatic activity to convert isopentenyl diphosphate and dimethylallyl phosphate to geranyl diphosphate.

Accordingly, geranyl diphosphate synthase activity is understood as meaning the amount of isopentenyl diphosphate and/or dimethylallyl phosphate reacted or amount of geranyl diphosphate formed in a certain time by the protein geranyl diphosphate synthase.

With an increased geranyl diphosphate synthase activity compared to the wild-type, in comparison with the wild-type, the amount of isopentenyl diphosphate and/or dimethylallyl phosphate reacted or the amount of geranyl diphosphate formed is thus increased in a certain time by the protein geranyl diphosphate synthase.

Preferably, this increase in the geranyl diphosphate synthase activity amounts to at least 5%, furthermore preferably at least 20%, furthermore preferably at least 50%, furthermore preferably at least 100%, more preferably at least 300%, even more preferably at least 500%, in particular at least 600%, of the geranyl diphosphate synthase activity of the wild-type.

The determination of the geranyl diphosphate synthase activity in genetically modified organisms according to the invention and in wild-type or reference organisms is preferably carried out under the following conditions:

Frozen organism material is homogenized by intensive grinding in liquid nitrogen in a mortar and pestle and extracted with extraction buffer in a ratio of 1:1 to 1:20. The particular ratio depends on the enzyme activities in the available organism material, such that a determination and quantification of the enzyme activities within the linear measurement range is possible. Typically, the extraction buffer can consist of 50 mM HEPES-KOH (pH 7.4), 10 mM MgCl₂, 10 mM KCl, 1 mM EDTA, 1 mM EGTA, 0.1% (v/v) Triton X-100, 2 mM ε-aminocaproic acid, 10% glycerol, 5 mM KHCO₃. Shortly before the extraction, 2 mM DTT and 0.5 mM PMSF are added.

The activity of the geranyl diphosphate synthase (GPP synthase) can be determined in 50 mM Tris-HCl (pH 7.6), 10 mM MgCl₂, 5 mM MnCl₂, 2 mM DTT, 1 mM ATP, 0.2% Tween-20, 5 μM (^{14C})IPP and 50 μM DMAPP (dimethylallyl pyrophosphate) after addition of organism extract (according to Bouvier, Suire, d'Harlingue, Backhaus and Camara: Molecular cloning of geranyl diphosphate synthase and compartmentation of monoterpene synthesis in plant cells, Plant Journal 24 (2000) 241-252). After the incubation of, for example, 2 hours at 37° C., the reaction products are dephosphyrylated (according to Koyama, Fuji and Ogura: Enzymatic hydrolysis of polyprenyl pyrophosphates, Methods Enzymol. 110 (1985), 153-155) and analyzed by means of thin layer chromatography and measurement of the incorporated radioactivity (Dogbo, Bardat, Quennemet and Camara: Metabolism of plastid terpenoids: In vitro inhibition of phytoene synthesis by phenethyl pyrophosphate derivates, FEBS Letters 219 (1987) 211-215).

Farnesyl diphosphate synthase activity is understood as meaning the enzyme activity of a farnesyl diphosphate synthase.

A farnesyl diphosphate synthase is understood as meaning a protein which has the enzymatic activity sequentially to convert 2 molecules of isopentenyl diphosphate with dimethylallyl diphosphate and the resulting geranyl diphosphate to farnesyl diphosphate.

Accordingly, farnesyl diphosphate synthase activity is understood as meaning the amount of dimethylallyl diphosphate and/or isopentenyl diphosphate reacted or amount of farnesyl diphosphate formed in a certain time by the protein farnesyl diphosphate synthase.

With an increased farnesyl diphosphate synthase activity compared to the wild-type, in comparison with the wild-type the amount of dimethylallyl diphosphate and/or isopentenyl diphosphate reacted or the amount of farnesyl diphosphate formed in a certain time is thus increased by the protein farnesyl diphosphate synthase.

Preferably, this increase in the farnesyl diphosphate synthase activity amounts to at least 5%, furthermore preferably at least 20%, furthermore preferably at least 50%, furthermore preferably at least 100%, more preferably at least 300%, even more preferably at least 500%, in particular at least 600%, of the farnesyl diphosphate synthase activity of the wild-type.

The determination of the farnesyl diphosphate synthase activity in genetically modified organisms according to the invention and in wild-type or reference organisms is preferably carried out under the following conditions:

Frozen organism material is homogenized by intensive grinding in liquid nitrogen in a mortar and pestle and extracted with extraction buffer in a ratio of 1:1 to 1:20. The particular ratio depends on the enzyme activities in the available organism material, such that a determination and quantification of the enzyme activities within the linear measurement range is possible. Typically, the extraction buffer can consist of 50 mM HEPES-KOH (pH 7.4), 10 mM MgCl₂, 10 mM KCl, 1 mM EDTA, 1 mM EGTA, 0.1% (v/v) Triton X-100, 2 mM ε-aminocaproic acid, 10% glycerol, 5 mM KHCO₃. Shortly before the extraction, 2 mM DTT and 0.5 mM PMSF are added.

The activity of the franesyl pyrophosphate snthase (FPP synthase) can be determined according to a procedure of Joly and Edwards (Journal of Biological Chemistry 268 (1993), 26983-26989). Afterwards, the enzyme activity is measured in a buffer consisting of 10 mM HEPES (pH 7.2), 1 mM MgCl₂, 1 mM dithiothreitol, 20 υM geranyl pyrophosphate and 40 μM (1-¹⁴C) isopentenyl pyrophosphate (4 Ci/mmol). The reaction mixture is incubated at 37° C.; the reaction is stopped by addition of 2.5 N HCl (in 70% ethanol with 19 μg/ml of farnesol). The reaction products are thus hydrolyzed within 30 minutes by acid hydrolysis at 37C. The mixture is neutralized by addition of 10% NaOH and extracted by shaking with hexane. An aliquot of the hexane phase can be measured by means of a scintillation counter for the determination of the incorporated radioactivity.

Alternatively, after incubation of organism extract and radiolabeled IPP, the reaction products are separated by thin layer chromatography (silica gel SE60, Merck) in benzene/methanol (9:1). Radiolabeled products are eluted and the radioactivity is determined (according to Gaffe, Bru, Causse, Vidal, Stamitti-Bert, Carde and Gallusci: LEFPS1, a tomato farnesyl pyrophosphate gene highly expressed during early fruit development; Plant Physiology 123 (2000) 1351-1362).

Geranylgeranyl diphosphate synthase activity is understood as meaning the enzyme activity of a geranylgeranyl diphosphate synthase.

A geranylgeranyl diphosphate synthase is understood as meaning a protein which has the enzymatic activity to convert farnesyl diphosphate and isopentenyl diphosphate to geranylgeranyl diphosphate.

Accordingly, a geranylgeranyl diphosphate synthase activity is understood as meaning the amount of farnesyl diphosphate and/or isopentenyl diphosphate reacted or amount of geranylgeranyl diphosphate formed in a certain time by the protein geranylgeranyl diphosphate synthase.

With an increased geranylgeranyl diphosphate synthase activity compared to the wild-type, in comparison with the wild-type the amount of farnesyl diphosphate and/or isopentenyl diphosphate reacted or the amount of geranylgeranyl diphosphate formed is thus increased in a certain time by the protein geranylgeranyl diphosphate synthase.

Preferably, this increase in the geranylgeranyl diphosphate synthase activity amounts to at least 5%, furthermore preferably at least 20%, furthermore preferably at least 50%, furthermore preferably at least 100%, more preferably at least 300%, even more preferably at least 500%, in particular at least 600%, of the geranylgeranyl piphosphate synthase activity of the wild-type.

The determination of the geranylgeranyl diphosphate synthase activity in genetically modified organisms according to the invention and in wild-type or reference organisms is preferably carried out under the following conditions:

Frozen organism material is homogenized by intensive grinding in liquid nitrogen in a mortar and pestle and extracted with extraction buffer in a ratio of 1:1 to 1:20. The particular ratio depends on the enzyme activities in the available organism material, such that a determination and quantification of the enzyme activities within the linear measurement range is possible. Typically, the extraction buffer can consist of 50 mM HEPES-KOH (pH 7.4), 10 mM MgCl₂, 10 mM KCl, 1 mM EDTA, 1 mM EGTA, 0.1% (v/v) Triton X-100, 2 mM ε-aminocaproic acid, 10% glycerol, 5 mM KHCO₃. Shortly before the extraction, 2 mM DTT and 0.5 mM PMSF are added.

Activity measurements of the geranylgeranyl pyrophosphate Synthase (GGPP synthase) can be determined according to the method described by Dogbo and Camara (in Biochim. Biophys. Acta 920 (1987), 140-148: Purification of isopentenyl pyrophosphate isomerase and geranylgeranyl pyrophosphate synthase from Capsicum chromoplasts by affinity chromatography). To this end, a buffer (50 mM Tris-HCl (pH 7.6), 2 mM MgCl₂, 1 mM MnCl₂, 2 mM Dithiothreitol, (1-¹⁴C)IPP (0.1 υCi, 10 ∝M), 15 ∝M DMAPP, GPP or FPP) is added with a total volume of approximately 200 υl of organism extract. Incubation can be carried out for 1-2 hours (or longer) at 30C. The reaction is by addition of 0.5 ml of ethanol and 0.1 ml of 6N HCl. After incubation at 37° C. for 10 minutes, the reaction mixture is neutralized with 6N NaOH, mixed with 1 ml of water and extracted by shaking with 4 ml of diethyl ether. In an aliquot (e.g. 0.2 ml) of the ether phase, the amount of radioactivity is determined by means of scintillation countering. Alternatively, after acid hydrolysis the radiolabeled prenyl alcohols can be extracted into ether by shaking and separated using HPLC (25 cm column of Spherisorb ODS-1, 5 υm; elution with methanol/water (90:10; v/v) at a flow rate of 1 ml/min) and quantified by means of a radioactivity monitor (according to Wiedemann, Misawa and Sandmann: Purification and enzymatic characterization of the geranylgeranyl pyrophosphate synthase from Erwinia uredovora after expression in Escherichia coli; Archives Biochemistry and Biophysics 306 (1993), 152-157).

Phytoene synthase activity is understood as meaning the enzyme activity of a phytoene synthase.

In particular, a phytoene synthase is understood as meaning a protein which has the enzymatic activity to convert geranylgeranyl diphosphate to phytoene.

Accordingly, phytoene synthase activity is understood as meaning the amount of geranylgeranyl diphosphate reacted or amount of phytoene formed in a certain time by the protein phytoene synthase.

With an increased phytoene synthase activity compared to the wild-type, in comparison with the wild-type the amount of geranylgeranyl diphosphate reacted or the amount of phytoene formed in a certain time is increased by the protein phytoene synthase.

Preferably, this increase in the phytoene synthase activity amounts to at least 5%, furthermore preferably at least 20%, furthermore preferably at least 50%, furthermore preferably at least 100%, more preferably at least 300%, even more preferably at least 500%, in particular at least 600%, of the phytoene synthase activity of the wild-type.

The determination of the phytoene synthase activity in genetically modified organisms according to the invention and in wild-type or reference organisms is preferably carried out under the following conditions:

Frozen organism material is homogenized by intensive grinding in liquid nitrogen in a mortar and pestle and extracted with extraction buffer in a ratio of 1:1 to 1:20. The particular ratio depends on the enzyme activities in the available organism material, such that a determination and quantification of the enzyme activities within the linear measurement range is possible. Typically, the extraction buffer can consist of 50 mM HEPES-KOH (pH 7.4), 10 mM MgCl₂, 10 mM KCl, 1 mM EDTA, 1 mM EGTA, 0.1% (v/v) Triton X-100, 2 mM ε-aminocaproic acid, 10% glycerol, 5 mM KHCO₃. Shortly before the extraction, 2 mM DTT and 0.5 mM PMSF are added.

Activity measurements of the phytoene synthase (PSY) can be carried out according to the method presented by Fraser and colleagues (Fraser, Romer, Shipton, Mills, Kiano, Misawa, Drake, Schuch and Bramley: Evaluation of transgenic tomato plants expressing an additional phytoene synthase in a fruit-specific manner; Proc. Natl. Acad. Sci. USA 99 (2002), 1092-1097, based on Fraser, Pinto, Holloway and Bramley, Plant Journal 24 (2000) 551-558). For enzyme measurements, incubations with (³H)geranylgeranyl pyrophosphate (15 mCi/mM, American Radiolabeled Chemicals, St. Louis) as a substrate are carried out in 0.4 M Tris-HCl (pH 8.0) with 1 mM DTT, 4 mM MgCl₂, 6 mM MnCl₂, 3 mM ATP, 0.1% Tween 60, 1 mM potassium fluoride. Organism extracts are mixed with buffer, e.g. 295 υl of buffer with extract in a total volume of 500 υl. The mixture is incubated for at least 5 hours at 28C. Subsequently, phytoene is extracted twice by shaking (in each case 500 ∝l) with chloroform. The radiolabeled phytoene formed during the reaction is separated by means of thin layer chromatography on silica plates in methanol/water (95:5; v/v). Phytoene can be identified on the silica plates in an iodine-enriched atmosphere (by heating a few iodine crystals). A phytoene standard is used as a reference. The amount of radiolabeled product is determined by means of measurements in the scintillation counter. Alternatively, phytoene can also be quantified by means of HPLC which is provided with a radioactivity detector (Fraser, Albrecht and Sandmann: Development of high performance liquid chromatographic systems for the separation of radiolabeled carotenes and precursors formed in specific enzymatic reactions; J. Chromatogr. 645 (1993) 265-272).

Phytoene desaturase activity is understood as meaning the enzyme activity of a phytoene desaturase.

A phytoene desaturase is understood as meaning a protein which has the enzymatic activity to convert phytoene to phytofluene and/or phytofluene to ζ-carotene (zetacarotene).

Accordingly, phytoene desaturase activity is understood as meaning the amount of phytoene or phytofluene reacted or amount of phytofluene or ζ-carotene formed in a certain time by the protein phytoene desaturase.

With an increased phytoene desaturase activity compared to the wild-type, in comparison with the wild-type the amount of phytoene or phytofluene reacted or the amount of phytofluene or ζ-carotene formed in a certain time is thus increased by the protein phytoene desaturase.

Preferably, this increase in the phytoene desaturase activity amounts to at least 5%, furthermore preferably at least 20%, furthermore preferably at least 50%, furthermore preferably at least 100%, more preferably at least 300%, even more preferably at least 500%, in particular at least 600%, of the phytoene desaturase activity of the wild-type.

The determination of the phytoene desaturase activity in genetically modified organisms according to the invention and in wild-type or reference organisms is preferably carried out under the following conditions:

Frozen organism material is homogenized by intensive grinding in liquid nitrogen in a mortar and pestle and extracted with extraction buffer in a ratio of 1:1 to 1:20. The particular ratio depends on the enzyme activities in the available organism material, such that a determination and quantification of the enzyme activities within the linear measurement range is possible. Typically, the extraction buffer can consist of 50 mM HEPES-KOH (pH 7.4), 10 mM MgCl₂, 10 mM KCl, 1 mM EDTA, 1 mM EGTA, 0.1% (v/v) Triton X-100, 2 mM ε-aminocaproic acid, 10% glycerol, 5 mM KHCO₃. Shortly before the extraction, 2 mM DTT and 0.5 mM PMSF are added.

The activity of the phytoene desaturase (PDS) can be measured by the insertion of radiolabeled (¹⁴C)-phytoene in unsaturated carotenes (according to Römer, Fraser, Kiano, Shipton, Misawa, Schuch and Bramley: Elevation of the provitamin A content of transgenic tomato plants; Nature Biotechnology 18 (2000) 666-669). Radiolabeled phytoene can be synthesized according to Fraser (Fraser, De la Rivas, Mackenzie, Bramley: Phycomyces blakesleanus CarB mutants: their use in assays of phytoene desaturase; Phytochemistry 30 (1991), 3971-3976). Membranes of plastids of the target tissue can be incubated with 100 mM MES buffer (pH 6.0) with 10 mM MgCl₂ and 1 mM dithiothreitol in a total volume 1 ml. (¹⁴C)-Phytoene dissolved in acetone (approximately 100 000 disintegrations/minute for in each case one incubation) is added, where the acetone concentration 5% (v/v) should not be exceeded. This mixture is incubated with shaking at 28C for approximately 6 to 7 hours in the dark. Afterwards, pigments are extracted three times with approximately 5 ml of petroleum ether (mixed with 10% diethyl ether) and separated and quantified by means of HPLC.

Alternatively, the activity of the phytoene desaturase can be measured according to Fraser et al. (Fraser, Misawa, Linden, Yamano, Kobayashi and Sandmann: Expression in Escherichia coli, purification, and reactivation of the recombinant Erwinia uredovora phytoene desaturase, Journal of Biological Chemistry 267 (1992) 19891-9895).

Zeta-carotene desaturase activity is understood as meaning the enzyme activity of a zeta-carotene desaturase.

A zeta-carotene desaturase is understood as meaning a protein which has the enzymatic activity to convert ζ-carotene to neurosporin and/or neurosporin to lycopene.

Accordingly, zeta-carotene desaturase activity is understood as meaning the amount of ζ-carotene or neurosporin reacted or amount of neurosporin or lycopene formed in a certain time by the protein zeta-carotene desaturase.

With an increased zeta-carotene desaturase activity compared to the wild-type, in comparison with the wild-type the amount of ζ-carotene or neurosporin reacted or the amount of neurosporin or lycopene formed in a certain time is increased by the protein zeta-carotene desaturase.

Preferably, this increase in the zeta-carotene desaturase activity amounts to at least 5%, furthermore preferably at least 20%, furthermore preferably at least 50%, furthermore preferably at least 100%, more preferably at least 300%, even more preferably at least 500%, in particular at least 600%, of the zeta-carotene desaturase activity of the wild-type.

The determination of the zeta-carotene desaturase activity in genetically modified organisms according to the invention and in wild-type or reference organisms is preferably carried out under the following conditions:

Frozen organism material is homogenized by intensive grinding in liquid nitrogen in a mortar and pestle and extracted with extraction buffer in a ratio of 1:1 to 1:20. The particular ratio depends on the enzyme activities in the available organism material, such that a determination and quantification of the enzyme activities within the linear measurement range is possible. Typically, the extraction buffer can consist of 50 mM HEPES-KOH (pH 7.4), 10 mM MgCl₂, 10 mM KCl, 1 mM EDTA, 1 mM EGTA, 0.1% (v/v) Triton X-100, 2 mM E-aminocaproic acid, 10% glycerol, 5 mM KHCO₃. Shortly before the extraction, 2 mM DTT and 0.5 mM PMSF are added.

Analyses for the determination of the ξ-carotene desaturase (ZDS desaturase) can be carried out in 0.2 M potassium phosphate (pH 7.8, buffer volume of approximately 1 ml). The analysis method for this was published by Breitenbach and colleagues (Breitenbach, Kuntz, Takaichi and Sandmann: Catalytic properties of an expressed and purified higher plant type ξ-carotene desaturase from Capsicum annuum; European Journal of Biochemistry. 265(1):376-383, 1999). Each analysis batch comprises 3 mg of phosphytidylcholine, which is suspended in 0.4 M potassium phosphate buffer (pH 7.8), 5 υg of ξ-carotene or neurosporin, 0.02% butylhydroxytoluene, 10 υl of decyl-plastoquinone (1 mM methanolic stock solution) and organism extract. The volume of the organism extract must be adjusted to the amount of ZDS desaturase activity present in order to make possible quantifications in a linear measurement range. Incubations are typically carried out for about 17 hours with vigorous shaking (200 revolutions/minute) at approximately 28° C. in the dark. Carotenoids are extracted with shaking by addition of 4 ml of acetone at 50° C. for 10 minutes. From this mixture, the carotenoids are transferred to a petroleum ether phase (with 10% to diethyl ether). The diethyl ether/petroleum ether phase is evaporated under nitrogen, and the carotenoids are dissolved again in 20 υl and separated and quantified by means of HPLC.

crtISO activity is understood as meaning the enzyme activity of a crtISO protein.

A crtISO protein is understood as meaning a protein which has the enzymatic activity to convert 7,9,7′,9′-tetra-cis-lycopene to all-trans-lycopene.

Accordingly, crtISO activity is understood as meaning the amount of 7,9,7′,9′-tetra-cis-lycopene reacted or amount of all-trans-lycopene formed in a certain time by the protein crtISO.

With an increased crtISO activity compared to the wild-type, in comparison with the wild-type the amount of 7,9,7′,9′-tetra-cis-lycopene reacted or the amount of all-trans-lycopene formed in a certain time is thus increased by the crtISO protein.

Preferably, this increase in the crtISO activity amounts to at least 5%, furthermore preferably at least 20%, furthermore preferably at least 50%, furthermore preferably at least 100%, more preferably at least 300%, even more preferably at least 500%, in particular at least 600%, of the crtISO activity of the wild-type.

FtsZ activity is understood as meaning the physiological activity of a FtsZ protein.

An FtsZ protein is understood as meaning a protein which has a cell division and plastid division-promoting action and has homologies to tubulin proteins.

MinD activity is understood as meaning the physiological activity of a MinD protein.

A MinD protein is understood as meaning a protein which has a multifunctional role in cell division. It is a membrane-associated ATPase and within the cell can show an oscillating motion from pole to pole.

Furthermore, the increase in the activity of enzymes of the non-mevalonate pathway can lead to a further increase in the desired ketocarotenoid final product. Examples of this are the 4-diphosphocytidyl-2-C-methyl-D-erythritol synthase, the 4-diphosphocytidyl-2-C-methyl-D-erythritol kinase and the 2-C-methyl-D-erythritol-2,4-cyclodiphoshate synthase. By modifications of the gene expression of the corresponding genes, the activity of the enzymes mentioned can be increased. The modified concentrations of the relavant proteins can be detected in a standard manner by means of antibodies and appropriate blotting techniques.

The increase in the HMG-CoA reductase activity and/or (E)-4-hydroxy-3-methylbut-2-enyl diphosphate reductase activity and/or 1-deoxy-D-xylose-5-phosphate synthase activity and/or 1-deoxy-D-xylose-5-phosphate reductoisomerase activity and/or isopentenyl diphosphate Δ-isomerase activity and/or geranyl diphosphate synthase activity and/or farnesyl diphosphate synthase activity and/or geranylgeranyl diphosphate synthase activity and/or phytoene synthase activity and/or phytoene desaturase activity and/or zeta-carotene desaturase activity and/or crtISO activity and/or FtsZ activity and/or MinD activity can be carried out in various ways, for example by switching off inhibitory regulation mechanisms at the expression and protein level or by increasing the gene expression of nucleic acids encoding an HMG-CoA reductase and/or nucleic acids encoding an (E)-4-hydroxy-3-methylbut-2-enyl diphosphate reductase and/or nucleic acids encoding a 1-deoxy-D-xylose-5-phosphate synthase and/or nucleic acids encoding a 1-deoxy-D-xylose-5-phosphate reductoisomerase and/or nucleic acids encoding an isopentenyl diphosphate Δ-isomerase and/or nucleic acids encoding a geranyl diphosphate synthase and/or nucleic acids encoding a farnesyl diphosphate synthase and/or nucleic acids encoding a geranylgeranyl diphosphate synthase and/or nucleic acids encoding a phytoene synthase and/or nucleic acids encoding a phytoene desaturase and/or nucleic acids encoding a zeta-carotene desaturase and/or nucleic acids encoding a crtISO protein and/or nucleic acids encoding a FtsZ protein and/or nucleic acids encoding a MinD protein compared to the wild-type.

The increase in the gene expression of the nucleic acids encoding an HMG-CoA reductase and/or nucleic acids encoding an (E)-4-hydroxy-3-methylbut-2-enyl diphosphate reductase and/or nucleic acids encoding a 1-deoxy-D-xylose-5-phosphate synthase and/or nucleic acids encoding a 1-deoxy-D-xylose-5-phosphate reductoisomerase and/or nucleic acids encoding an isopentenyl diphosphate Δ-isomerase and/or nucleic acids encoding a geranyl diphosphate synthase and/or nucleic acids encoding a farnesyl diphosphate synthase and/or nucleic acids encoding a geranylgeranyl diphosphate synthase and/or nucleic acids encoding a phytoene synthase and/or nucleic acids encoding a phytoene desaturase and/or nucleic acids encoding a zeta-carotene desaturase and/or nucleic acids encoding a crtISO protein and/or nucleic acids encoding a FtsZ protein and/or nucleic acids encoding a MinD protein compared to the wild-type can likewise be carried out in various ways, for example by induction of the HMG-CoA reductase gene and/or (E)-4-hydroxy-3-methylbut-2-enyl diphosphate reductase gene and/or 1-deoxy-D-xylose-5-phosphate synthase gene and/or 1-deoxy-D-xylose-5-phosphate reductoisomerase gene and/or isopentenyl diphosphate Δ-isomerase gene and/or geranyl diphosphate synthase gene and/or farnesyl diphosphate synthase gene and/or geranylgeranyl diphosphate synthase gene and/or phytoene synthase gene and/or phytoene desaturase gene and/or zeta-carotene desaturase gene and/or crtISO gene and/or FtsZ gene and/or MinD gene by activators or by insertion of one or more copies of the HMG-CoA reductase gene and/or (E)₄-hydroxy-3-methylbut-2-enyl diphosphate reductase gene and/or 1-deoxy-D-xylose-5-phosphate synthase gene and/or 1-deoxy-D-xylose-5-phosphate reductoisomerase gene and/or isopentenyl diphosphate Δ-isomerase gene and/or geranyl diphosphate synthase gene and/or farnesyl diphosphate synthase gene and/or geranylgeranyl diphosphate synthase gene and/or phytoene synthase gene and/or phytoene desaturase gene and/or zeta-carotene desaturase gene and/or crtISO gene and/or FtsZ gene and/or MinD gene, that is by insertion of at least one nucleic acid encoding an HMG-CoA reductase and/or at least one nucleic acid encoding an (E)-4-hydroxy-3-methylbut-2-enyl diphosphate reductase and/or at least one nucleic acid encoding a 1-deoxy-D-xylose-5-phosphate synthase and/or at least one nucleic acid encoding a 1-deoxy-D-xylose-5-phosphate reductoisomerase and/or at least one nucleic acid encoding an isopentenyl diphosphate Δ-isomerase and/or at least one nucleic acid encoding a geranyl diphosphate synthase and/or at least one nucleic acid encoding a farnesyl diphosphate synthase and/or at least one nucleic acid encoding a geranylgeranyl diphosphate synthase and/or at least one nucleic acid encoding a phytoene synthase and/or at least one nucleic acid encoding a phytoene desaturase and/or at least one nucleic acid encoding a zeta-carotene desaturase and/or at least one nucleic acid encoding a crtISO protein and/or at least one nucleic acid encoding an FtsZ protein and/or at least one nucleic acid encoding a MinD protein into the plant.

Increase in the gene expression of a nucleic acid encoding an HMG-CoA reductase and/or (E)-4-hydroxy-3-methylbut-2-enyl diphosphate reductase and/or 1-deoxy-D-xylose-5-phosphate synthase and/or 1-deoxy-D-xylose-5-phosphate reductoisomerase and/or isopentenyl diphosphate Δ-isomerase and/or geranyl diphosphate synthase and/or farnesyl diphosphate synthase and/or geranylgeranyl diphosphate synthase and/or phytoene synthase and/or phytoene desaturase and/or zeta-carotene desaturase and/or a crtISO protein and/or FtsZ protein and/or MinD protein is understood according to the invention as also meaning the manipulation of the expression of the organism's own, endogenous HMG-CoA reductase and/or (E)-4-hydroxy-3-methylbut-2-enyl diphosphate reductase and/or 1-deoxy-D-xylose-5-phosphate synthase and/or 1-deoxy-D-xylose-5-phosphate reductoisomerase and/or isopentenyl diphosphate Δ-isomerase and/or geranyl diphosphate synthase and/or farnesyl diphosphate synthase and/or geranylgeranyl diphosphate synthase and/or phytoene synthase and/or phytoene desaturase and/or zeta-carotene desaturase and/or of the organism's own crtISO protein and/or FtsZ protein and/or MinD protein.

This can be achieved, for example, by modification of the corresponding promoter DNA sequence. Such a modification, which results in an increased expression rate of the gene, can be carried out, for example, by deletion or insertion of DNA sequences.

In a preferred embodiment, the increase in the gene expression of a nucleic acid encoding an HMG-CoA reductase and/or the increase in the gene expression of a nucleic acid encoding an (E)-4-hydroxy-3-methylbut-2-enyl diphosphate reductase and/or the increase in the gene expression of a nucleic acid encoding a 1-deoxy-D-xylose-5-phosphate synthase and/or the increase in the gene expression of a nucleic acid encoding a 1-deoxy-D-xylose-5-phosphate reductoisomerase and/or the increase in the gene expression of a nucleic acid encoding an isopentenyl diphosphate Δ-isomerase and/or the increase in the gene expression of a nucleic acid encoding a geranyl diphosphate synthase and/or the increase in the gene expression of a nucleic acid encoding a farnesyl diphosphate synthase and/or the increase in the gene expression of a nucleic acid encoding a geranylgeranyl diphosphate synthase and/or the increase in the gene expression of a nucleic acid encoding a phytoene synthase and/or the increase in the gene expression of a nucleic acid encoding a phytoene desaturase and/or the increase in the gene expression of a nucleic acid encoding a zeta-carotene desaturase and/or the increase in the gene expression of a nucleic acid encoding an crtISO protein and/or the increase in the gene expression of a nucleic acid encoding a FtsZ protein and/or the increase in the gene expression of a nucleic acid encoding an MinD protein by insertion of at least one nucleic acid encoding an HMG-CoA reductase and/or by insertion of at least one nucleic acid encoding an (E)-4-hydroxy-3-methylbut-2-enyl diphosphate reductase and/or by insertion of at least one nucleic acid encoding a 1-deoxy-D-xylose-5-phosphate synthase and/or by insertion of at least one nucleic acid encoding a 1-deoxy-D-xylose-5-phosphate reductoisomerase and/or by insertion of at least one nucleic acid encoding an isopentenyl diphosphate Δ-isomerase and/or by insertion of at least one nucleic acid encoding a geranyl diphosphate synthase and/or by insertion of at least one nucleic acid encoding a farnesyl diphosphate synthase and/or by insertion of at least one nucleic acid encoding a geranylgeranyl diphosphate synthase and/or by insertion of at least one nucleic acid encoding a phytoene synthase and/or by insertion of at least one nucleic acid encoding a phytoene desaturase and/or by insertion of at least one nucleic acid encoding a zeta-carotene desaturase and/or by insertion of at least one nucleic acid encoding a crtISO protein and/or by insertion of at least one nucleic acid encoding an FtsZ protein and/or by insertion of at least one nucleic acid encoding an MinD protein into the plant.

To this end, in principle any HMG-CoA reductase gene or (E)-4-hydroxy-3-methylbut-2-enyl diphosphate reductase gene or 1-deoxy-D-xylose-5-phosphate synthase gene or 1-deoxy-D-xylose-5-phosphate reductoisomerase gene or isopentenyl diphosphate Δ-isomerase gene or geranyl diphosphate synthase gene or farnesyl diphosphate synthase gene or geranylgeranyl diphosphate synthase gene or phytoene synthase gene or phytoene desaturase gene or zeta-carotene desaturase gene or crtISO gene or FtsZ gene or MinD gene can be used.

With genomic HMG-CoA reductase sequences or (E)-4-hydroxy-3-methylbut-2-enyl diphosphate reductase sequences or 1-deoxy-D-xylose-5-phosphate synthase sequences or 1-deoxy-D-xylose-5-phosphate reductoisomerase sequences or isopentenyl diphosphate Δ-isomerase sequences or geranyl diphosphate synthase sequences or farnesyl diphosphate synthase sequences or geranylgeranyl diphosphate synthase sequences or phytoene synthase sequences or phytoene desaturase sequences or zeta-carotene desaturase sequences or crtISO sequences or FtsZ sequences or MinD sequences from eukaryotic sources which comprise introns, in the case where the host plant is not in the position or cannot be put in the position of expressing the corresponding proteins, preferably already processed nucleic acid sequences, such as the corresponding cDNAs, are to be used.

In the preferred transgenic organisms according to the invention, in this preferred embodiment compared to the wild-type at least one further HMG-CoA reductase gene and/or (E)-4-hydroxy-3-methylbut-2-enyl diphosphate reductase gene and/or 1-deoxy-D-xylose-5-phosphate synthase gene and/or 1-deoxy-D-xylose-5-phosphate reductoisomerase gene and/or isopentenyl diphosphate Δ-isomerase gene and/or geranyl diphosphate synthase gene and/or farnesyl diphosphate synthase gene and/or geranylgeranyl diphosphate synthase gene and/or phytoene synthase gene and/or phytoene desaturase gene and/or zeta-carotene desaturase gene and/or crtISO gene and/or FtsZ gene and/or MinD gene is present.

In this preferred embodiment, the genetically modified plant, for example, has at least one exogenous nucleic acid encoding an HMG-CoA reductase or at least two endogenous nucleic acids encoding an HMG-CoA reductase and/or at least one exogenous nucleic acid encoding an (E)-4-hydroxy-3-methylbut-2-enyl diphosphate reductase or at least two endogenous nucleic acids encoding an (E)-4-hydroxy-3-methylbut-2-enyl diphosphate reductase and/or at least one exogenous nucleic acid encoding a 1-deoxy-D-xylose-5-phosphate synthase or at least two endogenous nucleic acids encoding a 1-deoxy-D-xylose-5-phosphate synthase and/or at least one exogenous nucleic acid encoding a 1-deoxy-D-xylose-5-phosphate reductoisomerase or at least two endogenous nucleic acids encoding a 1-deoxy-D-xylose-5-phosphate reductoisomerase and/or at least one exogenous nucleic acid encoding an isopentenyl diphosphate Δ-isomerase or at least two endogenous nucleic acids encoding an isopentenyl diphosphate Δ-isomerase and/or at least one exogenous nucleic acid encoding a geranyl diphosphate synthase or at least two endogenous nucleic acids encoding a geranyl diphosphate synthase and/or at least one exogenous nucleic acid encoding a farnesyl diphosphate synthase or at least two endogenous nucleic acids encoding a farnesyl diphosphate synthase and/or at least one exogenous nucleic acid encoding a geranylgeranyl diphosphate synthase or at least two endogenous nucleic acids encoding a geranylgeranyl diphosphate synthase and/or at least one exogenous nucleic acid encoding a phytoene synthase or at least two endogenous nucleic acids encoding a phytoene synthase and/or at least one exogenous nucleic acid encoding a phytoene desaturase or at least two endogenous nucleic acids encoding a phytoene desaturase and/or at least one exogenous nucleic acid encoding a zeta-carotene desaturase or at least two endogenous nucleic acids encoding a zeta-carotene desaturase and/or at least one exogenous nucleic acid encoding a crtISO protein or at least two endogenous nucleic acids encoding a crtISO protein and/or at least one exogenous nucleic acid encoding an FtsZ protein or at least two endogenous nucleic acids encoding an FtsZ protein and/or at least one exogenous nucleic acid encoding a MinD protein or at least two endogenous nucleic acids, encoding an MinD protein.

Examples of HMG-CoA reductase genes are:

a nucleic acid encoding an HMG-CoA reductase from Arabidopsis thaliana, Accession NM_{—}106299; (nucleic acid: SEQ ID NO: 7, protein: SEQ ID NO: 8),

and further HMG-CoA reductase genes from other organisms with the following accession numbers:

P54961, P54870, P54868, P54869, O02734, P22791, P54873, P54871, P23228, P13704, P54872, Q01581, P17425, P54874, P54839, P14891, P34135, O64966, P29057, P48019, P48020, P12683, P43256, Q9XEL8, P34136, O64967, P29058, P48022, Q41437, P12684, Q00583, Q9XHL5, Q41438, Q9YAS4, O76819, O28538, Q9Y7D2, P54960, O51628, P48021, Q03163, P00347, P14773, Q12577, Q59468, P04035, O24594, P09610, Q58116, O26662, Q01237, Q01559, Q12649, O74164, O59469, P51639, Q10283, O08424, P20715, P13703, P13702, Q96UG4, Q8SQZ9, O15888, Q9TUM4, P93514, Q39628, P93081, P93080, Q944T9, Q40148, Q84MM0, Q84LS3, Q9Z9N4, Q9KLM0

Examples of (E)-4-hydroxy-3-methylbut-2-enyl diphosphate reductase genes are:

a nucleic acid encoding an (E)-4-hydroxy-3-methylbut-2-enyl diphosphate reductase from Arabidopsis thaliana (lytB/ISPH), ACCESSION AY168881, (nucleic acid: SEQ ID NO: 9, protein: SEQ ID NO:102),

and further (E)-4-hydroxy-3-methylbut-2-enyl diphosphate reductase genes from other organs with the following accession numbers:

T04781, AF270978_{—}1, NP_{—}485028.1, NP_{—}442089.1, NP_{—}681832.1, ZP_{—}00110421.1, ZP_{—}00071594.1, ZP_{—}00114706.1, ISPH_SYNY3, ZP_{—}00114087.1, ZP_{—}00104269.1, AF398145_{—}1, AF398146_{—}1, AAD55762.1, AF514843_{—}1, NP_{—}622970.1, NP_{—}348471.1, NP_{—}562001.1, NP_{—}223698.1, NP_{—}781941.1, ZP_{—}00080042.1, NP_{—}859669.1, NP_{—}214191.1, ZP_{—}00086191.1, ISPH_VIBCH, NP_{—}230334.1, NP_{—}742768.1, NP_{—}302306.1, ISPH_MYCLE, NP_{—}602581.1, ZP_{—}00026966.1, NP_{—}520563.1, NP_{—}253247.1, NP_{—}282047.1, ZP_{—}00038210.1, ZP_{—}00064913.1, CAA61555.1, ZP_{—}00125365.1, ISPH_ACICA, EAA24703.1, ZP_{—}00013067.1, ZP_{—}00029164.1, NP_{—}790656.1, NP_{—}217899.1, NP_{—}641592.1, NP_{—}636532.1, NP_{—}719076.1, NP_{—}660497.1, NP_{—}422155.1, NP_{—}715446.1, ZP_{—}00090692.1, NP_{—}759496.1, ISPH_BURPS, ZP_{—}00129657.1, NP_{—}215626.1, NP_{—}335584.1, ZP_{—}00135016.1, NP_{—}789585.1, NP_{—}787770.1, NP_{—}769647.1, ZP_{—}00043336.1, NP_{—}242248.1, ZP_{—}00008555.1, NP_{—}246603.1, ZP_{—}00030951.1, NP_{—}670994.1, NP_{—}404120.1, NP_{—}540376.1, NP_{—}733653.1, NP_{—}697503.1, NP_{—}840730.1, NP_{—}274828.1, NP_{—}796916.1, ZP_{—}00123390.1, NP_{—}824386.1, NP_{—}737689.1, ZP_{—}00021222.1, NP_{—}757521.1, NP_{—}390395.1, ZP_{—}00133322.1, CAD76178.1, NP_{—}600249.1, NP_{—}454660.1, NP_{—}712601.1, NP_{—}385018.1, NP_{—}751989.1

Examples of 1-deoxy-D-xylose-5-phosphate synthase genes are:

a nucleic acid encoding a 1-deoxy-D-xylose-5-phosphate synthase from Lycopersicon esculentum, ACCESSION #AF143812 (nucleic acid: SEQ ID NO:103, protein: SEQ ID NO: 12),

and further 1-deoxy-D-xylose-5-phosphate synthase genes from other organisms with the following accession numbers:

AF143812_{—}1, DXS_CAPAN, CAD22530.1, AF182286_{—}1, NP_{—}193291.1, T52289, AAC49368.1, AAP14353.1, D71420, DXS_ORYSA, AF443590_{—}1, BAB02345.1, CAA09804.2, NP_{—}850620.1, CAD22155.2, AAM65798.1, NP_{—}566686.1, CAD22531.1, AAC33513.1, CAC08458.1, AAG10432.1, T08140, AAP14354.1, AF428463_{—}1, ZP_{—}00010537.1, NP_{—}769291.1, AAK59424.1, NP_{—}107784.1, NP_{—}697464.1, NP_{—}540415.1, NP_{—}196699.1, NP_{—}384986.1, ZP_{—}00096461.1, ZP_{—}00013656.1, NP_{—}353769.1, BAA83576.1, ZP_{—}00005919.1, ZP_{—}00006273.1, NP_{—}420871.1, AAM48660.1, DXS_RHOCA, ZP_{—}00045608.1, ZP_{—}00031686.1, NP_{—}841218.1, ZP_{—}00022174.1, ZP_{—}00086851.1, NP_{—}742690.1, NP_{—}520342.1, ZP_{—}00082120.1, NP_{—}790545.1, ZP_{—}00125266.1, CAC17468.1, NP_{—}252733.1, ZP_{—}00092466.1, NP_{—}439591.1, NP_{—}414954.1, NP_{—}752465.1, NP_{—}622918.1, NP_{—}286162.1, NP_{—}836085.1, NP_{—}706308.1, ZP_{—}00081148.1, NP_{—}797065.1, NP_{—}213598.1, NP_{—}245469.1, ZP_{—}00075029.1, NP_{—}455016.1, NP_{—}230536.1, NP_{—}459417.1, NP_{—}274863.1, NP_{—}283402.1, NP_{—}759318.1, NP_{—}406652.1, DXS_SYNLE, DXS_SYNP7, NP_{—}440409.1, ZP_{—}00067331.1, ZP_{—}00122853.1, NP_{—}717142.1, ZP_{—}00104889.1, NP_{—}243645.1, NP_{—}681412.1, DXS_SYNEL, NP_{—}637787.1, DXS_CHLTE, ZP_{—}00129863.1, NP_{—}661241.1, DXS_XANCP, NP_{—}470738.1, NP_{—}484643.1, ZP_{—}00108360.1, NP_{—}833890.1, NP_{—}846629.1, NP_{—}658213.1, NP_{—}642879.1, ZP_{—}00039479.1, ZP_{—}00060584.1, ZP_{—}00041364.1, ZP_{—}00117779.1, NP_{—}299528.1

Examples of 1-deoxy-D-xylose-5-phosphate reductoisomerase genes are:

a nucleic acid encoding an 1-deoxy-D-xylose-5-phosphate reductoisomerase from Arabidopsis thaliana, ACCESSION #AF148852, (nucleic acid: SEQ ID NO: 13, protein: SEQ ID NO: 14),

and further 1-deoxy-D-xylose-5-phosphate reductoisomerase genes from other organisms with the following accession numbers:

AF148852, AY084775, AY054682, AY050802, AY045634, AY081453, AY091405, AY098952, AJ242588, AB009053, AY202991, NP_{—}201085.1, T52570, AF331705_{—}1, BAB16915.1, AF367205_{—}1, AF250235_{—}1, CAC03581.1, CAD22156.1, AF182287_{—}1, DXR_MENP₁, ZP_{—}00071219.1, NP_{—}488391.1, ZP_{—}00111307.1, DXR_SYNLE, AAP56260.1, NP_{—}681831.1, NP_{—}442113.1, ZP_{—}00115071.1, ZP_{—}00105106.1, ZP_{—}00113484.1, NP_{—}833540.1, NP_{—}657789.1, NP_{—}661031.1, DXR_BACHD, NP_{—}833080.1, NP_{—}845693.1, NP_{—}562610.1, NP_{—}623020.1, NP_{—}810915.1, NP_{—}243287.1, ZP_{—}00118743.1, NP_{—}464842.1, NP_{—}470690.1, ZP_{—}00082201.1, NP_{—}781898.1, ZP_{—}00123667.1, NP_{—}348420.1, NP_{—}604221.1, ZP_{—}00053349.1, ZP_{—}00064941.1, NP_{—}246927.1, NP_{—}389537.1, ZP_{—}00102576.1, NP_{—}519531.1, AF124757_{—}19, DXR_ZYMMO, NP_{—}713472.1, NP_{—}459225.1, NP_{—}454827.1, ZP_{—}00045738.1, NP_{—}743754.1, DXR_PSEPK, ZP_{—}00130352.1, NP_{—}702530.1, NP_{—}841744.1, NP_{—}438967.1, AF514841_{—}1, NP_{—}706118.1, ZP_{—}00125845.1, NP_{—}404661.1, NP_{—}285867.1, NP_{—}240064.1, NP_{—}414715.1, ZP_{—}00094058.1, NP_{—}791365.1, ZP_{—}00012448.1, ZP_{—}00015132.1, ZP_{—}00091545.1, NP_{—}629822.1, NP_{—}771495.1, NP_{—}798691.1, NP_{—}231885.1, NP_{—}252340.1, ZP_{—}00022353.1, NP_{—}355549.1, NP_{—}420724.1, ZP_{—}00085169.1, EAA17616.1, NP_{—}273242.1, NP_{—}219574.1, NP_{—}387094.1, NP_{—}296721.1, ZP_{—}00004209.1, NP_{—}823739.1, NP_{—}282934.1, BAA77848.1, NP_{—}660577.1, NP_{—}760741.1, NP_{—}641750.1, NP_{—}636741.1, NP_{—}829309.1, NP_{—}298338.1, NP_{—}444964.1, NP_{—}717246.1, NP_{—}224545.1, ZP_{—}00038451.1, DXR_KITGR, NP_{—}778563.1.

Examples of isopentenyl diphosphate Δ-isomerase genes are:

a nucleic acid encoding an isopentenyl diphosphate Δ-isomerase from Adonis palaestina clone AplPI28, (ipiAa1), ACCESSION #AF188060, published by Cunningham, F. X. Jr. and Gantt, E.: Identification of multi-gene families encoding isopentenyl diphosphate isomerase in plants by heterologous complementation in Escherichia coli, Plant Cell Physiol. 41 (1), 119-123 (2000) (nucleic acid: SEQ ID NO: 15, protein: SEQ ID NO: 16),

and further isopentenyl diphosphate Δ-isomerase genes from other organisms with the following accession numbers:

Q38929, O48964, Q39472, Q13907, O35586, P58044, O42641, O35760, Q10132, P15496, Q9YB30, Q8YNH4, Q42553, O27997, P50740, O51627, O48965, Q8KFR5, Q39471, Q39664, Q9RVE2, Q01335, Q9HHE4, Q9BXS1, Q9KWF6, Q9CIF5, Q88WB6, Q92BX2, Q8Y7A5, Q8TT35Q9KK75, Q8NN99, Q8XD58, Q8FE75, Q46822, Q9HP40, P72002, P26173, Q9Z5D3, Q8Z3X9, Q8ZM82, Q9X7Q6, O13504, Q9HFW8, Q8NJL9, Q9UUQ1, Q9NHO2, Q9M6K9, Q9M6K5, Q9FXR6, O81691, Q9S7C4, Q8S3L8, Q9M592, Q9M6K3, Q9M6K7, Q9FV48, Q9LLB6, Q9AVJ1, Q9AVG8, Q9M6K6, Q9AVJ5, Q9M6K2, Q9AYS5, Q9M6K8, Q9AVG7, Q8S3L7, Q8W250, Q941E1, Q9AVI8, Q9AYS6, Q9SAY0, Q9M6K4, Q8GVZ0, Q84RZ8, Q8KZ12, Q8KZ66, Q8FND7, Q88QC9, Q8BFZ6, BAC26382, CAD94476.

Examples of geranyl diphosphate synthase genes are:

a nucleic acid encoding a geranyl diphosphate synthase from Arabidopsis thaliana, ACCESSION #Y17376, Bouvier, F., Suire, C., d'Harlingue, A., Backhaus, R. A. and Camara, B.; Molecular cloning of geranyl phosphate synthase and compartmentation of monoterpene synthesis in plant cells, Plant J. 24 (2), 241-252 (2000) (nucleic acid: SEQ ID NO: 17, protein: SEQ ID NO: 18),

and further geranyl diphosphate synthase genes from other organisms with the following accession numbers:

Q9FT89, Q8LKJ2, Q9FSW8, Q8LKJ3, Q9SBR3, Q9SBR4, Q9FET8, Q8LKJ1, Q84LG1, Q9JK86

Examples of farnesyl diphosphate synthase genes are:

a nucleic acid encoding a farnesyl diphosphate synthase from Arabidopsis thaliana (FPS1), ACCESSION #U80605, published by Cunillera, N., Arro, M., Delourme, D., Karst, F., Boronat, A. and Ferrer, A.: Arabidopsis thaliana contains two differentially expressed farnesyl phosphate synthase genes, J. Biol. Chem. 271 (13), 7774-7780 (1996), (nucleic acid: SEQ ID NO: 19, protein: SEQ ID NO:112),

and further farnesyl diphosphate synthase genes from other organisms with the following accession numbers:

P53799, P37268, Q02769, Q09152, P49351, O24241, Q43315, P49352, O24242, P49350, P08836, P14324, P49349, P08524, O66952, Q08291, P54383, Q45220, P57537, Q8K9A0, P22939, P45204, O66126, P55539, Q9SWH9, Q9AVI7, Q9FRX2, Q9AYS7, Q941E8, Q9FXR9, Q9ZWF6, Q9FXR8, Q9AR37, O50009, Q941E9, Q8RVK7, Q8RVQ7, O04882, Q93RA8, Q93RB0, Q93RB4, Q93RB5, Q93RB3, Q93RB1, Q93RB2, Q920E5.

Examples of geranylgeranyl diphosphate synthase genes are:

a nucleic acid encoding a geranylgeranyl diphosphate synthase from Sinaps alba, ACCESSION #X98795, published by Bonk, M., Hoffmann, B., Von Lintig, J., Schledz, M., Al-Babili, S., Hobeika, E., Kleinig, H. and Beyer, P.: Chloroplast import of four carotenoid biosynthetic enzymes in vitro reveals differential fates prior to membrane binding and oligomeric assembly, Eur. J. Biochem. 247 (3), 942-950 (1997), (nucleic acid: SEQ ID NO: 21, protein: SEQ ID NO:114),

and further geranylgeranyl diphosphate synthase genes from other organisms with the following accession numbers:

P22873, P34802, P56966, P80042, Q42698, Q92236, O95749, Q9WTN0, Q50727, P24322, P39464, Q9FXR3, Q9AYN2, Q9FXR2, Q9AVG6, Q9FRW4, Q9SXZ5, Q9AVJ7, Q9AYN1, Q9AVJ4, Q9FXR7, Q8LSC5, Q9AVJ6, Q8LSC4, Q9AVJ3, Q9SSU0, Q9SXZ6, Q9SST9, Q9AVJ0, Q9AVI9, Q9FRW3, Q9FXR5, Q941F0, Q9FRX1, Q9K567, Q93RA9, Q93QX8, CAD95619, EAA31459

Examples of phytoene synthase genes are:

a nucleic acid encoding a phytoene synthase from Erwinia uredovora, ACCESSION # D90087; published by Misawa, N., Nakagawa, M., Kobayashi, K., Yamano, S., Izawa, Y., Nakamura, K. and Harashima, K.: Elucidation of the Erwinia uredovora carotenoid biosynthetic pathway by functional analysis of gene products expressed in Escherichia coli; J. Bacteriol. 172 (12), 6704-6712 (1990), (nucleic acid: SEQ ID NO: 23, protein: SEQ ID NO: 24),

and further phytoene synthase genes from other organisms with the following accession numbers:

CAB39693, BAC69364, AAF10440, CAA45350, BAA20384, AAM72615, BAC09112, CAA48922, P_{—}001091, CAB84588, AAF41518, CAA48155, AAD38051, AAF33237, AAG10427, AAA34187, BAB73532, CAC19567, AAM62787, CAA55391, AAB65697, AAM45379, CAC27383, AAA32836, AAK07735, AAM84763, P_{—}000205, AAB60314, P_{—}001163, P_{—}000718, AAB71428, AAA34153, AAK07734, CAA42969, CAD76176, CAA68575, P_{—}000130, P_{—}001142, CAA47625, CAA85775, BAC14416, CAA79957, BAC76563, P_{—}000242, AAL_{—}000551, AAL02001, AAK15621, CAB94795, AAA91951, P_{—}000448

Examples of phytoene desaturase genes are:

a nucleic acid encoding a phytoene desaturase from Erwinia uredovora, ACCESSION # D90087; published by Misawa, N., Nakagawa, M., Kobayashi, K., Yamano, S., Izawa, Y., Nakamura, K. and Harashima, K.: Elucidation of the Erwinia uredovora carotenoid biosynthetic pathway by functional analysis of gene products expressed in Escherichia coli; J. Bacteriol. 172 (12), 6704-6712 (1990), (nucleic acid: SEQ ID NO: 25, protein: SEQ ID NO: 26),

and further phytoene desaturase genes from other organisms with the following accession numbers:

AAL15300, A39597, CAA42573, AAK51545, BAB08179, CAA48195, BAB82461, AAK92625, CAA55392, AAG10426, AAD02489, AAO24235, AAC12846, AAA99519, AAL38046, CAA60479, CAA75094, ZP_{—}001041, ZP_{—}001163, CAA39004, CAA44452, ZP_{—}001142, ZP_{—}000718, BAB82462, AAM45380, CAB56040, ZP_{—}001091, BAC09113, AAP79175, AAL80005, AAM72642, AAM72043, ZP_{—}000745, ZP_{—}001141, BAC07889, CAD55814, ZP_{—}001041, CAD27442, CAE00192, ZP_{—}001163, ZP_{—}000197, BAA18400, AAG10425, ZP_{—}001119, AAF13698, 2121278A, AAB35386, AAD02462, BAB68552, CAC85667, AAK51557, CAA12062, AAG51402, AAM63349, AAF85796, BAB74081, AAA91161, CAB56041, AAC48983, AAG14399, CAB65434, BAB73487, ZP_{—}001117, ZP_{—}000448, CAB39695, CAD76175, BAC69363, AAM17934, ZP_{—}000171, AAF65586, ZP_{—}000748, BAC07074, ZP_{—}001133, AAM64853, BAB74484, ZP_{—}001156, AAF23289, AAG28703, AAP09348, AAM71569, BAB69140, ZP_{—}000130, AAF41516, AAG18866, CAD95940, NP_{—}656310, AAG10645, ZP_{—}000276, ZP_{—}000192, ZP_{—}000186, AAM94364, AAM31371, ZP_{—}000612, BAC75676, AAF65582

Examples of zeta-carotene desaturase genes are:

a nucleic acid encoding a zeta-carotene desaturase from Narcissus pseudonarcissus, ACCESSION #AJ224683, published by Al-Babili, S., Oelschlegel, J. and Beyer, P.: A cDNA encoding for beta carotene desaturase (Accession No. AJ224683) from Narcissus pseudonarcissus L. (PGR98-103), Plant Physiol. 117, 719-719 (1998), (nucleic acid: SEQ ID NO: 119, protein: SEQ ID NO: 28),

and further zeta-carotene desaturase genes from other organisms with the following accession numbers:

Q9R6×4, Q38893, Q9SMJ3, Q9SE20, Q9ZTP4, O49901, P74306, Q9FV46, Q9RCT2, ZDS_NARPS, BAB68552.1, CAC85667.1, AF372617_{—}1, ZDS_TARER, CAD55814.1, CAD27442.1, 2121278A, ZDS_CAPAN, ZDS_LYCES, NP_{—}187138.1, AAM63349.1, ZDS_ARATH, AAA91161.1, ZDS_MAIZE, AAG14399.1, NP_{—}441720.1, NP_{—}486422.1, ZP_{—}00111920.1, CAB56041.1, ZP_{—}00074512.1, ZP_{—}00116357.1, NP_{—}681127.1, ZP_{—}00114185.1, ZP_{—}00104126.1, CAB65434.1, NP_{—}662300.1

Examples of crtISO genes are:

a nucleic acid encoding a crtISO from Lycopersicon esculentum; ACCESSION #AF416727, published by Isaacson, T., Ronen, G., Zamir, D. and Hirschberg, J.: Cloning of tangerine from tomato reveals a carotenoid isomerase essential for the production of beta-carotene and xanthophylls in plants; Plant Cell 14 (2), 333-342 (2002), (nucleic acid: SEQ ID NO: 29, protein: SEQ ID NO:122),

and further crtISO genes from other organisms with the following accession numbers:

AAM53952

Examples of FtsZ genes are:

a nucleic acid encoding an FtsZ from Tagetes erecta, ACCESSION #AF251346, published by Moehs, C. P., Tian, L., Osteryoung, K. W. and Dellapenna, D.: Analysis of carotenoid biosynthetic gene expression during marigold petal development Plant Mol. Biol. 45 (3), 281-293 (2001), (nucleic acid: SEQ ID NO: 31, protein: SEQ ID NO: 32),

and further FtsZ genes from other organisms with the following accession numbers:

CAB89286.1, AF205858_{—}1, NP_{—}200339.1, CAB89287.1, CAB41987.1, AAA82068.1, T06774, AF383876_{—}1, BAC57986.1, CAD22047.1, BAB91150.1, ZP_{—}00072546.1, NP_{—}440816.1, T51092, NP_{—}683172.1, BAA85116.1, NP_{—}487898.1, JC4289, BAA82871.1, NP_{—}781763.1, BAC57987.1, ZP_{—}00111461.1, T51088, NP_{—}190843.1, ZP_{—}00060035.1, NP_{—}846285.1, AAL07180.1, NP_{—}243424.1, NP_{—}833626.1, AAN04561.1, AAN04557.1, CAD22048.1, T51089, NP_{—}692394.1, NP_{—}623237.1, NP_{—}565839.1, T51090, CAA07676.1, NP_{—}113397.1, T51087, CAC44257.1, E84778, ZP_{—}00105267.1, BAA82091.1, ZP_{—}00112790.1, BAA96782.1, NP_{—}348319.1, NP_{—}471472.1, ZP_{—}00115870.1, NP_{—}465556.1, NP_{—}389412.1, BAA82090.1, NP_{—}562681.1, AAM22891.1, NP_{—}371710.1, NP_{—}764416.1, CAB95028.1, FTSZ_STRGR, AF120117_{—}1, NP_{—}827300.1, JE0282, NP_{—}626341.1, AAC45639.1, NP_{—}785689.1, NP_{—}336679.1, NP_{—}738660.1, ZP_{—}00057764.1, AAC32265.1, NP_{—}814733.1, FTSZ_MYCKA, NP_{—}216666.1, AAM75616.1, NP_{—}301700.1, NP_{—}601357.1, ZP_{—}00046269.1, AAM70158.1, ZP_{—}00037834.1, NP_{—}268026.1, FTSZ_ENTHR, NP_{—}787643.1, NP_{—}346105.1, AAC32264.1, JC5548, AAC95440.1, NP_{—}710793.1, NP_{—}687509.1, NP_{—}269594.1, AAC32266.1, NP_{—}720988.1, NP_{—}657875.1, ZP_{—}00094865.1, ZP_{—}00080499.1, ZP_{—}00043589.1, JC7087, NP_{—}660559.1, AAC46069.1, AF179611_{—}14, AAC44223.1, NP_{—}404201.1.

Examples of MinD genes are:

a nucleic acid encoding a MinD from Tagetes erecta, ACCESSION #AF251019, published by Moehs, C. P., Tian, L., Osteryoung, K. W. and Dellapenna, D.: Analysis of carotenoid biosynthetic gene expression during marigold petal development; Plant Mol. Biol. 45 (3), 281-293 (2001), (nucleic acid: SEQ ID NO: 33, protein: SEQ ID NO: 34),

and further MinD genes with the following accession numbers:

NP_{—}197790.1, BAA90628.1, NP_{—}038435.1, NP_{—}045875.1, AAN33031.1, NP_{—}050910.1, CAB53105.1, NP_{—}050687.1, NP_{—}682807.1, NP_{—}487496.1, ZP_{—}00111708.1, ZP_{—}00071109.1, NP_{—}442592.1, NP_{—}603083.1, NP_{—}782631.1, ZP_{—}00097367.1, ZP_{—}00104319.1, NP_{—}294476.1, NP_{—}622555.1, NP_{—}563054.1, NP_{—}347881.1, ZP_{—}00113908.1, NP_{—}834154.1, NP_{—}658480.1, ZP_{—}00059858.1, NP_{—}470915.1, NP_{—}243893.1, NP_{—}465069.1, ZP_{—}00116155.1, NP_{—}390677.1, NP_{—}692970.1, NP_{—}298610.1, NP_{—}207129.1, ZP_{—}00038874.1, NP_{—}778791.1, NP_{—}223033.1, NP_{—}641561.1, NP_{—}636499.1, ZP_{—}00088714.1, NP_{—}213595.1, NP_{—}743889.1, NP_{—}231594.1, ZP_{—}00085067.1, NP_{—}797252.1, ZP_{—}00136593.1, NP_{—}251934.1, NP_{—}405629.1, NP_{—}759144.1, ZP_{—}00102939.1, NP_{—}793645.1, NP_{—}699517.1, NP_{—}460771.1, NP_{—}860754.1, NP_{—}456322.1, NP_{—}718163.1, NP_{—}229666.1, NP_{—}357356.1, NP_{—}541904.1, NP_{—}287414.1, NP_{—}660660.1, ZP_{—}00128273.1, NP_{—}103411.1, NP_{—}785789.1, NP_{—}715361.1, AF149810_{—}1, NP_{—}841854.1, NP_{—}437893.1, ZP_{—}00022726.1, EAA24844.1, ZP_{—}00029547.1, NP_{—}521484.1, NP_{—}240148.1, NP_{—}770852.1, AF345908_{—}2, NP_{—}777923.1, ZP_{—}00048879.1, NP_{—}579340.1, NP_{—}143455.1, NP_{—}126254.1, NP_{—}142573.1, NP_{—}613505.1, NP_{—}127112.1, NP_{—}712786.1, NP_{—}578214.1, NP_{—}069530.1, NP_{—}247526.1, AAA85593.1, NP_{—}212403.1, NP_{—}782258.1, ZP_{—}00058694.1, NP_{—}247137.1, NP_{—}219149.1, NP_{—}276946.1, NP_{—}614522.1, ZP_{—}00019288.1, CAD78330.1

Preferably, in the preferred embodiment described above the HMG-CoA reductase genes used are nucleic acids which encode proteins comprising the amino acid sequence SEQ ID NO: 8 or a sequence derived from this sequence by substitution, insertion or deletion of amino acids, which has an identity of at least 30%, preferably at least 50%, more preferably at least 70%, even more preferably at least 90%, most preferably at least 95%, at the amino acid level with the sequence SEQ ID NO: 8, and which have the enzymatic properties of an HMG-CoA reductase.

Further examples of HMG-CoA reductases and HMG-CoA reductase genes can easily be found, for example, from various organisms whose genomic sequence is known, as described above, by homology comparisons of the amino acid sequences or of the corresponding back-translated nucleic acid sequences from databases containing the SeQ ID NO: 8.

Further examples of HMG-CoA reductases and HMG-CoA reductase genes can furthermore easily be found, for example, starting from the sequence SEQ ID NO: 7 from various organisms whose genomic sequence is not known, as described above, by hybridization and PCR techniques in a manner known per se.

In a furthermore particularly preferred embodiment, for increasing the HMG-CoA reductase activity nucleic acids are inserted into organisms which encode proteins comprising the amino acid sequence of the HMG-CoA reductase of the sequence SEQ ID NO: 8.

Suitable nucleic acid sequences are obtainable, for example, by back-translation of the polypeptide sequence according to the genetic code.

Preferably, those codons are used for this which are often used according to the organism-specific codon usage. The codon usage can easily be determined with the aid of computer analyses of other, known genes of the organisms concerned.

In a particularly preferred embodiment, a nucleic acid comprising the sequence SEQ ID NO: 7 is inserted into the organism.

Preferably, in the preferred embodiment described above the (E)-4-hydroxy-3-methylbut-2-enyl diphosphate reductase genes used are nucleic acids which encode proteins comprising the amino acid sequence SEQ ID NO: 10 or a sequence derived from this sequence by substitution, insertion or deletion of amino acids, which has an identity of at least 30%, preferably at least 50%, more preferably at least 70%, even more preferably at least 90%, most preferably at least 95% at the amino acid level with the sequence SEQ ID NO: 10, and which have the enzymatic properties of an (E)-4-hydroxy-3-methylbut-2-enyl diphosphate reductase.

Further examples of (E)-4-hydroxy-3-methylbut-2-enyl diphosphate reductases and (E)-4-hydroxy-3-methylbut-2-enyl diphosphate reductase genes can easily be found, for example, from various organisms whose genomic sequence is known, as described above, by homology comparisons of the amino acid sequences or of the corresponding back-translated nucleic acid sequences from databases containing the SeQ ID NO: 10.

Further examples of (E)-4-hydroxy-3-methylbut-2-enyl diphosphate reductases and (E)-4-hydroxy-3-methylbut-2-enyl diphosphate reductase genes can furthermore easily be found, for example, starting from the sequence SEQ ID NO: 9 from various organisms whose genomic sequence is not known, as described above, by hybridization and PCR techniques in a manner known per se.

In a further particularly preferred embodiment, for increasing the (E)₄-hydroxy-3-methylbut-2-enyl diphosphate reductase activity nucleic acids are inserted in organisms which encode proteins comprising the amino acid sequence of the (E)-4-hydroxy-3-methylbut-2-enyl diphosphate reductase of the sequence SEQ ID NO: 10.

Suitable nucleic acid sequences are obtainable, for example, by back-translation of the polypeptide sequence according to the genetic code.

Preferably, those codons are used for this which are often used according to the organism-specific codon usage. The codon usage can easily be determined with the aid of computer analyses of other, known genes of the organisms concerned.

In a particularly preferred embodiment, a nucleic acid comprising the sequence SEQ ID NO: 9 is inserted into the organism.

Preferably, in the preferred embodiment described above the (1-deoxy-D-xylose-5-phosphate synthase genes used are nucleic acids which encode proteins comprising the amino acid sequence SEQ ID NO: 12 or a sequence derived from this sequence by substitution, insertion or deletion of amino acids, which have an identity of at least 30%, preferably at least 50%, more preferably at least 70%, even more preferably at least 90%, most preferably at least 95% at the amino acid level with the sequence SEQ ID NO: 12, and which have the enzymatic properties of a (1-deoxy-D-xylose-5-phosphate synthase.

Further examples of (1-deoxy-D-xylose-5-phosphate synthases and (1-deoxy-D-xylose-5-phosphate synthase genes can easily be found, for example, from various organisms whose genomic sequence is known, as described above, by homology comparisons of the amino acid sequences or of the corresponding back-translated nucleic acid sequences from databases containing the SeQ ID NO: 12.

Further examples of (1-deoxy-D-xylose-5-phosphate synthases and (1-deoxy-D-xylose-5-phosphate synthase genes can furthermore easily be found, for example, starting from the sequence SEQ ID NO: 11 of various organisms whose genomic sequence is not known, as described above, by hybridization and PCR techniques in a manner known per se.

In a furthermore particularly preferred embodiment, for increasing the (1-deoxy-D-xylose-5-phosphate synthase activity nucleic acids are inserted into organisms which encode proteins comprising the amino acid sequence of the (1-deoxy-D-xylose-5-phosphate synthase of the sequence SEQ ID NO: 12.

Suitable nucleic acid sequences are obtainable, for example, by back-translation of the polypeptide sequence according to the genetic code.

Preferably, those codons are used for this which are often used according to the organism-specific codon usage. The codon usage can easily be determined with the aid of computer analyses of other, known genes of the organisms concerned.

In a particularly preferred embodiment, a nucleic acid comprising the sequence SEQ ID NO: 11 is inserted into the organism.

Preferably, in the preferred embodiment described above the 1-deoxy-D-xylose-5-phosphate reductoisomerase genes used are nucleic acids which encode proteins comprising the amino acid sequence SEQ ID NO: 14 or a sequence derived from this sequence by substitution, insertion or deletion of amino acids, which has an identity of at least 30%, preferably at least 50%, more preferably at least 70%, even more preferably at least 90%, most preferably at least 95% at the amino acid level with the sequence SEQ ID NO: 14, and which have the enzymatic properties of a 1-deoxy-D-xylose-5-phosphate reductoisomerase.

Further examples of 1-deoxy-D-xylose-5-phosphate reductoisomerases and 1-deoxy-D-xylose-5-phosphate reductoisomerase genes can easily be found, for example, from various organisms whose genomic sequence is known, as described above, by homology comparisons of the amino acid sequences or of the corresponding back-translated nucleic acid sequences from databases containing the SeQ ID NO: 14.

Further examples of 1-deoxy-D-xylose-5-phosphate reductoisomerases and 1-deoxy-D-xylose-5-phosphate reductoisomerase genes can furthermore easily be found, for example, starting from the sequence SEQ ID NO: 13 of various organisms whose genomic sequence is not known, as described above, by hybridization and PCR techniques in a manner known per se.

In a furthermore particularly preferred embodiment, for increasing the 1-deoxy-D-xylose-5-phosphate reductoisomerase activity nucleic acids are inserted into organisms which encode proteins comprising the amino acid sequence of the 1-deoxy-D-xylose-5-phosphate reductoisomerase of the sequence SEQ ID NO: 14.

Suitable nucleic acid sequences are obtainable, for example, by back-translation of the polypeptide sequence according to the genetic code.

Preferably, those codons are used for this which are often used according to the organism-specific codon usage. The codon usage can easily be determined with the aid of computer analyses of other, known genes of the organisms concerned.

In a particularly preferred embodiment, a nucleic acid comprising the sequence SEQ ID NO: 13 is inserted into the organism.

Preferably, in the preferred embodiment described above the isopentenyl-D-isomerase genes used are nucleic acids which encode proteins comprising the amino acid sequence SEQ ID NO: 16 or a sequence derived from this sequence by substitution, insertion or deletion of amino acids, which have an identity of at least 30%, preferably at least 50%, more preferably at least 70%, even more preferably at least 90%, most preferably at least 95% at the amino acid level with the sequence SEQ ID NO: 16, and which have the enzymatic properties of an isopentenyl-D-isomerase.

Further examples of isopentenyl-D-isomerases and isopentenyl-D-isomerase genes can easily be found, for example, from various organisms whose genomic sequence is known, as described above, by homology comparisons of the amino acid sequences or of the corresponding back-translated nucleic acid sequences from databases containing the SeQ ID NO: 16.

Further examples of isopentenyl-D-isomerases and isopentenyl-D-isomerase genes can furthermore be easily discovered, for example, starting from the sequence SEQ ID NO: 15 of various organisms whose genomic sequence is not known, as described above, by hybridization and PCR techniques in a manner known per se.

In a further particularly preferred embodiment, for increasing the isopentenyl-D-isomerase activity nucleic acids are inserted into organisms which encode proteins comprising the amino acid sequence of the isopentenyl-D-isomerase of the sequence SEQ ID NO: 16.

Suitable nucleic acid sequences are obtainable, for example, by back-translation of the polypeptide sequence according to the genetic code.

Preferably, those codons are used for this which are often used according to the organism-specific codon usage. The codon usage can be easily determined with the aid of computer analyses of other, known genes of the organisms concerned.

In a particularly preferred embodiment, a nucleic acid comprising the sequence SEQ ID NO: 15 is inserted into the organism.

Preferably, in the preferred embodiment described above the geranyl diphosphate synthase genes used are nucleic acids which encode proteins comprising the amino acid sequence SEQ ID NO: 18 or a sequence derived from this sequence by substitution, insertion or deletion of amino acids, which have an identity of at least 30%, preferably at least 50%, more preferably at least 70%, even more preferably at least 90%, most preferably at least 95% at the amino acid level with the sequence SEQ ID NO: 18, and which have the enzymatic properties of a geranyl diphosphate synthase.

Further examples of geranyl diphosphate synthases and geranyl diphosphate synthase genes can easily be found, for example, from various organisms whose genomic sequence is known, as described above, by homology comparisons of the amino acid sequences or of the corresponding back-translated nucleic acid sequences from databases containing the SeQ ID NO: 18.

Further examples of geranyl diphosphate synthases and geranyl diphosphate synthase genes can furthermore be easily found, for example, starting from the sequence SEQ ID NO: 17 of various organisms whose genomic sequence is not known, as described above, by hybridization and PCR techniques in a manner known per se.

In a furthermore particularly preferred embodiment, for increasing the geranyl diphosphate synthase activity nucleic acids are inserted into organisms which encode proteins comprising the amino acid sequence of the geranyl diphosphate synthase of the sequence SEQ ID NO: 18.

Suitable nucleic acid sequences are obtainable, for example, by back-translation of the polypeptide sequence according to the genetic code.

Preferably, those codons are used for this which are often used according to the organism-specific codon usage. The codon usage can be easily determined with the aid of computer analyses of other, known genes of the organisms concerned.

In a particularly preferred embodiment, a nucleic acid comprising the sequence SEQ ID NO: 17 is inserted into the organism.

Preferably, in the preferred embodiment described above the farnesyl diphosphate synthase genes used are nucleic acids which encode proteins comprising the amino acid sequence SEQ ID NO: 20 or a sequence derived from this sequence by substitution, insertion or deletion of amino acids, which have an identity of at least 30%, preferably at least 50%, more preferably at least 70%, even more preferably at least 90%, most preferably at least 95% at the amino acid level with the sequence SEQ ID NO: 20, and have the enzymatic properties of a farnesyl diphosphate synthase.

Further examples of farnesyl diphosphate synthases and farnesyl diphosphate synthase genes can easily be determined, for example, from various organisms whose genomic sequence is known, as described above, by homology comparisons of the amino acid sequences or of the corresponding back-translated nucleic acid sequences from databases containing the SeQ ID NO: 20.

Further examples of farnesyl diphosphate synthases and farnesyl diphosphate synthase genes can furthermore be easily found, for example, starting from the sequence SEQ ID NO: 19 of various organisms whose genomic sequence is not known, as described above, by hybridization and PCR techniques in a manner known per se.

In a furthermore particularly preferred embodiment, for increasing the farnesyl diphosphate synthase activity nucleic acids are inserted into organisms which encode proteins comprising the amino acid sequence of the farnesyl diphosphate synthase of the sequence SEQ ID NO: 20.

Suitable nucleic acid sequences are obtainable, for example, by back-translation of the polypeptide sequence according to the genetic code.

Preferably, those codons are used for this which are often used according to the organism-specific codon usage. The codon usage can easily be determined with the aid of computer analyses of other, known genes of the organisms concerned.

In a particularly preferred embodiment, a nucleic acid comprising the sequence SEQ ID NO: 19 is inserted into the organism.

Preferably, in the preferred embodiment described above the geranylgeranyl diphosphate synthase genes used are nucleic acids which encode proteins comprising the amino acid sequence SEQ ID NO: 22 or a sequence derived from this sequence by substitution, insertion or deletion of amino acids, which have an identity of at least 30%, preferably at least 50%, more preferably at least 70%, even more preferably at least 90%, most preferably at least 95%, at the amino acid level with the sequence SEQ ID NO: 22, and which have the enzymatic properties of a geranylgeranyl diphosphate synthase.

Further examples of geranylgeranyl diphosphate synthases and geranylgeranyl diphosphate synthase genes can easily be found, for example, from various organisms whose genomic sequence is known, as described above, by homology comparisons of the amino acid sequences or of the corresponding back-translated nucleic acid sequences from databases containing the SeQ ID NO: 22.

Further examples of geranylgeranyl diphosphate synthases and geranylgeranyl diphosphate synthase genes can furthermore easily be found, for example, starting from the sequence SEQ ID NO: 21 of various organisms whose genomic sequence is not known, as described above, by hybridization and PCR techniques in a manner known per se.

In a furthermore particularly preferred embodiment, for increasing the geranylgeranyl diphosphate synthase activity nucleic acids are inserted into organisms which encode proteins comprising the amino acid sequence of the geranylgeranyl diphosphate synthase of the sequence SEQ ID NO: 22.

Suitable nucleic acid sequences are obtainable, for example, by back-translation of the polypeptide sequence according to the genetic code.

Preferably, those codons are used for this which are often used according to the organism-specific codon usage. The codon usage can be easily determined with the aid of computer analyses of other, known genes of the organisms concerned.

In a particularly preferred embodiment, a nucleic acid comprising the sequence SEQ ID NO: 21 is inserted into the organism.

Preferably, in the preferred embodiment described above the phytoene synthase genes used are nucleic acids which encode proteins comprising the amino acid sequence SEQ ID NO: 24 or a sequence derived from this sequence by substitution, insertion or deletion of amino acids, which have an identity of at least 30%, preferably at least 50%, more preferably at least 70%, even more preferably at least 90%, most preferably at least 95%, at the amino acid level with the sequence SEQ ID NO: 24, and which have the enzymatic properties of a phytoene synthase.

Further examples of phytoene synthases and phytoene synthase genes can easily be found, for example, from various organisms whose genomic sequence is known, as described above, by homology comparisons of the amino acid sequences or of the corresponding back-translated nucleic acid sequences from databases containing the SeQ ID NO: 24.

Further examples of phytoene synthases and phytoene synthase genes can furthermore easily be found, for example, starting from the sequence SEQ ID NO: 23 of various organisms whose genomic sequence is not known, as described above, by hybridization and PCR techniques in a manner known per se.

In a further particularly preferred embodiment, for increasing the phytoene synthase activity nucleic acids are inserted into organisms which encode proteins comprising the amino acid sequence of the phytoene synthase of the sequence SEQ ID NO: 24.

Suitable nucleic acid sequences are obtainable, for example, by back-translation of the polypeptide sequence according to the genetic code.

Preferably, those codons are used for this which are often used according to the organism-specific codon usage. The codon usage can easily be determined with the aid of computer analyses of other, known genes of the organisms concerned.

In a particularly preferred embodiment, a nucleic acid comprising the sequence SEQ ID NO: 23 is inserted into the organism.

Preferably, in the preferred embodiment described above the phytoene desaturase genes used are nucleic acids which encode proteins comprising the amino acid sequence SEQ ID NO: 26 or a sequence derived from this sequence by substitution, insertion or deletion of amino acids, which have an identity of at least 30%, preferably at least 50%, more preferably at least 70%, even more preferably at least 90%, most preferably at least 95%, at the amino acid level with the sequence SEQ ID NO: 26, and which have the enzymatic properties of a phytoene desaturase.

Further examples of phytoene desaturases and phytoene desaturase genes can easily be found, for example, from various organisms whose genomic sequence is known, as described above, by homology comparisons of the amino acid sequences or of the corresponding back-translated nucleic acid sequences from databases containing the SeQ ID NO: 26.

Further examples of phytoene desaturases and phytoene desaturase genes can furthermore easily be found starting from the sequence SEQ ID NO: 25 of various organisms whose genomic sequence is not known, as described above, by hybridization and PCR techniques in a manner known per se.

In a further particularly preferred embodiment, for increasing the phytoene desaturase activity nucleic acids are inserted into organisms which encode proteins comprising the amino acid sequence of the phytoene desaturase of the sequence SEQ ID NO: 26.

Suitable nucleic acid sequences are obtainable, for example, by back-translation of the polypeptide sequence according to the genetic code.

Preferably, codons are used for this which are often used according to the organism-specific codon usage. The codon usage can easily be determined with the aid of computer analyses of other, known genes of the organisms concerned.

In a particularly preferred embodiment, a nucleic acid comprising the sequence SEQ ID NO: 25 is inserted into the organism.

Preferably, in the preferred embodiment described above the zeta-carotene desaturase genes used are nucleic acids which encode proteins comprising the amino acid sequence SEQ ID NO: 28 or a sequence derived from this sequence by substitution, insertion or deletion of amino acids, which have an identity of at least 30%, preferably at least 50%, more preferably at least 70%, even more preferably at least 90%, most preferably at least 95%, at the amino acid level with the sequence SEQ ID NO: 28, and which have the enzymatic properties of a zeta-carotene desaturase.

Further examples of zeta-carotene desaturases and zeta-carotene desaturase genes can easily be found, for example, from various organisms, whose genomic sequence is known, as described above, by homology comparisons of the amino acid sequences or of the corresponding back-translated nucleic acid sequences from databases containing the SEQ ID NO: 28.

Further examples of zeta-carotene desaturases and zeta-carotene desaturase genes can furthermore easily be found, for example, starting from the sequence SEQ ID NO: 119 of various organisms whose genomic sequence is not known, as described above, by hybridization and PCR techniques in a manner known per se.

In a furthermore particularly preferred embodiment, for increasing the zeta-carotene desaturase activity nucleic acids are inserted into organisms which encode proteins comprising the amino acid sequence of the zeta-carotene desaturase of the sequence SEQ ID NO: 28.

Suitable nucleic acid sequences are obtainable, for example, by back-translation of the polypeptide sequence according to the genetic code.

Preferably, codons are used for this which are often used according to the organism-specific codon usage. The codon usage can easily be determined with the aid of computer analyses of other, known genes of the organisms concerned.

In a particularly preferred embodiment a nucleic acid comprising the sequence SEQ ID NO: 119 is inserted into the organism.

Preferably, in the preferred embodiment described above the CrtISO genes used are nucleic acids which encode proteins comprising the amino acid sequence SEQ ID NO: 30 or a sequence derived from this sequence by substitution, insertion or deletion of amino acids, which have an identity of at least 30%, preferably at least 50%, more preferably at least 70%, even more preferably at least 90%, most preferably at least 95%, at the amino acid level with the sequence SEQ ID NO: 30, and which have the enzymatic properties of a CrtIso.

Further examples of CrtISO and CrtISO genes can easily be found, for example, from various organisms whose genomic sequence is known, as described above, by homology comparisons of the amino acid sequences or of the corresponding back-translated nucleic acid sequences from databases containing the SeQ ID NO: 30.

Further examples of CrtISO and CrtISO genes can furthermore easily be found, for example, starting from the sequence SEQ ID NO: 29 of various organisms whose genomic sequence is not known, as described above, by hybridization and PCR techniques in a manner known per se.

In a further particularly preferred embodiment, for increasing the CrtISO activity nucleic acids are inserted into organisms which encode proteins comprising the amino acid sequence of the CrtISO of the sequence SEQ ID NO: 30.

Suitable nucleic acid sequences are obtainable, for example, by back-translation of the polypeptide sequence according to the genetic code.

Preferably, those codons are used for this which are often used according to the organism-specific codon usage. The codon usage can easily be determined with the aid of computer analyses of other, known genes of the organisms concerned.

In a particularly preferred embodiment, a nucleic acid comprising the sequence SEQ ID NO: 29 is inserted into the organism.

Preferably, in the preferred embodiment described above the FtsZ genes used are nucleic acids which encode proteins comprising the amino acid sequence SEQ ID NO: 32 or a sequence derived from this sequence by substitution, insertion or deletion of amino acids, which have an identity of at least 30%, preferably at least 50%, more preferably at least 70%, even more preferably at least 90%, most preferably at least 95%, at the amino acid level with the sequence SEQ ID NO: 32, and and which have the enzymatic properties of an FtsZ.

Further examples of FtsZn and FtsZ genes can easily be found, for example, from various organisms whose genomic sequence is known, as described above, by homology comparisons of the amino acid sequences or of the corresponding back-translated nucleic acid sequences from databases containing the SeQ ID NO: 32.

Further examples of FtsZn and FtsZ genes can furthermore easily be found, for example, starting from the sequence SEQ ID NO: 31 of various organisms whose genomic sequence is not known, as described above, by hybridization and PCR techniques in a manner known per se.

In a furthermore particularly preferred embodiment, for increasing the FtsZ activity nucleic acids are inserted into organisms which encode proteins comprising the amino acid sequence of the FtsZ of the sequence SEQ ID NO: 32

Suitable nucleic acid sequences are obtainable, for example, by back-translation of the polypeptide sequence according to the genetic code.

Preferably, those codons are used for this which are often used according to the organism-specific codon usage. The codon usage can be easily determined with the aid of computer analyses of other, known genes of the organisms concerned.

In a particularly preferred embodiment a nucleic acid comprising the sequence SEQ ID NO: 31 is inserted into the organism.

Preferably, in the preferred embodiment described above the MinD genes used are nucleic acids which encode proteins comprising the amino acid sequence SEQ ID NO: 34 or a sequence derived from this sequence by substitution, insertion or deletion of amino acids, which have an identity of at least 30%, preferably at least 50%, more preferably at least 70%, even more preferably at least 90%, most preferably at least 95%, at the amino acid level with the sequence SEQ ID NO: 34, and which have the enzymatic property of an MinD.

Further examples of MinDn and MinD genes can easily be found, for example, from various organisms whose genomic sequence is known, as described above, by homology comparisons of the amino acid sequences or of the corresponding back-translated nucleic acid sequences from databases containing the SeQ ID NO: 34.

Further examples of MinDn and MinD genes can furthermore easily be found, for example, starting from the sequence SEQ ID NO: 33 of various organisms whose genomic sequence is not known, as described above, by hybridization and PCR techniques in a manner known per se.

In a further particularly preferred embodiment, for increasing the MinD activity nucleic acids are inserted into organisms which encode proteins comprising the amino acid sequence of the MinD of the sequence SEQ ID NO: 34.

Suitable nucleic acid sequences are obtainable, for example, by back-translation of the polypeptide sequence according to the genetic code.

Preferably, those codons are used for this which are often used according to the organism-specific codon usage. The codon usage can easily be determined with the aid of computer analyses of other, known genes of the organisms concerned.

In a particularly preferred embodiment a nucleic acid comprising the sequence SEQ ID NO: 33 is inserted into the organism.

All abovementioned HMG-CoA reductase genes, (E)-4-hydroxy-3-methylbut-2-enyl diphosphate reductase genes, 1-deoxy-D-xylose-5-phosphate synthase genes, 1-deoxy-D-xylose-5-phosphate reductoisomerase genes, isopentenyl diphosphate Δ-isomerase genes, geranyl diphosphate synthase genes, farnesyl diphosphate synthase genes, geranylgeranyl diphosphate synthase genes, phytoene synthase genes, phytoene desaturase genes, zeta-carotene desaturase genes, crtISO genes, FtsZ genes or MinD genes can furthermore be prepared in a manner known per se by chemical synthesis from the nucleotide structural units, such as, for example, by fragment condensation of individual overlapping, complementary nucleic acid structural units of the double helix. The chemical synthesis of oligonucleotides can be carried out, for example, in a known manner, according to the phosphoamidite method (Voet, Voet, 2nd edition, Wiley Press New York, pages 896-897). The addition of synthetic oligonucleotides and filling of gaps with the aid of the Klenow fragment of the DNA polymerase and ligation reactions and general cloning processes are described in Sambrook et al. (1989), Molecular cloning: A laboratory manual, Cold Spring Harbor Laboratory Press.

The nucleic acids encoding a ketolase, nucleic acids encoding a β-hydroxylase, nucleic acids encoding a β-cyclase, comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2, and the nucleic acids encoding an HMG-CoA reductase, nucleic acids encoding an (E)-4-hydroxy-3-methylbut-2-enyl diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl diphosphate Δ-isomerase, nucleic acids encoding a geranyl diphosphate synthase, nucleic acids encoding a farnesyl diphosphate synthase, nucleic acids encoding a geranylgeranyl diphosphate synthase, nucleic acids encoding a phytoene synthase, nucleic acids encoding a phytoene desaturase, nucleic acids encoding a zeta-carotene desaturase, nucleic acids encoding a crtISO protein, nucleic acids encoding an FtsZ protein and/or nucleic acids encoding an MinD protein are also called “effect genes” below.

The production of the genetically modified, nonhuman organisms can be carried out, as described below, for example, by insertion of individual nucleic acid constructs (expression cassettes), comprising an effect gene or by insertion of multiple constructs, which comprise up to two or three of the effect genes or more than three effect genes.

Organisms are understood according to the invention as preferably meaning organisms which, as wild-type or starting organisms, naturally or by means of genetic complementation and/or reregulation of the metabolic pathways are in the position to produce carotenoids, in particular β-carotene and/or zeaxanthin and/or neoxanthin and/or violaxanthin and/or lutein.

Further preferred organisms, as wild-type or starting organisms, already have a hydroxylase activity and are thus, as wild-type or starting organisms, in the position to produce zeaxanthin.

Preferred organisms are plants or microorganisms, such as, for example, bacteria, yeasts, algae or fungi.

The bacteria used can be both bacteria which, on account of the insertion of genes of carotenoid biosynthesis of a carotenoid-producing organism are in the position to synthesize xanthophylls, such as, for example, bacteria of the genus Escherichia, which, for example, comprise crt genes from Erwinia, and bacteria which on their part are in the position to synthesize xanthophylls, such as, for example, bacteria of the genus Erwinia, Agrobacterium, Flavobacterium, Alcaligenes, Paracoccus, Nostoc or cyanobacteria of the genus Synechocystis.

Preferred bacteria are Escherichia coli, Erwinia herbicola, Erwinia uredovora, Agrobacterium aurantiacum, Alcaligenes sp. PC-1, Flavobacterium sp. strain R1534, the cyanobacterium Synechocystis sp. PCC6803, Paracoccus marcusii or Paracoccus caroteneifaciens.

Preferred yeasts are Candida, Saccharomyces, Hansenula, Pichia or Phaffia. Particularly preferred yeasts are Xanthophyllomyces dendrorhous or Phaffia rhodozyma.

Preferred fungi are Aspergillus, Trichoderma, Ashbya, Neurospora, Blakeslea, in particular Blakeslea trispora, Phycomyces, Fusarium or further fungi described in Indian Chem. Engr. Section B. Vol. 37, No. 1, 2 (1995) on page 15, Table 6.

Preferred algae are green algae, such as, for example, algae of the genus Haematococcus, Phaedactylum tricornatum, Volvox or Dunaliella. Particularly preferred algae are Haematococcus puvialis or Dunaliella bardawil.

Further utilizable microorganisms and their production for carrying out the process according to the invention are known, for example, from DE-A-199 16 140, to which reference is hereby made.

Particularly preferred plants are plants selected from the families Amaranthaceae, Amaryllidaceae, Apocynaceae, Asteraceae, Balsaminaceae, Begoniaceae, Berberidaceae, Brassicaceae, Cannabaceae, Caprifoliaceae, Caryophyllaceae, Chenopodiaceae, Compositae, Cucurbitaceae, Cruciferae, Euphorbiaceae, Fabaceae, Gentianaceae, Geraniaceae, Graminae, Illiaceae, Labiatae, Lamiaceae, Leguminosae, Liliaceae, Linaceae, Lobeliaceae, Malvaceae, Oleaceae, Orchidaceae, Papaveraceae, Plumbaginaceae, Poaceae, Polemoniaceae, Primulaceae, Ranunculaceae, Rosaceae, Rubiaceae, Scrophulariaceae, Solanaceae, Tropaeolaceae, Umbelliferae, Verbanaceae, Vitaceae and Violaceae.

Very particularly preferred plants are selected from the group consisting of the plant genera Marigold, Tagetes errecta, Tagetes patula, Acacia, Aconitum, Adonis, Arnica, Aquilegia, Aster, Astragalus, Bignonia, Calendula, Caltha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hisbiscus, Heliopsis, Hypericum, Hypochoeris, Impatiens, Iris, Jacaranda, Kerria, Laburnum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Sorbus, Spartium, Tecoma, Torenia, Tragopogon, Trollius, Tropaeolum, Tulipa, Tussilago, Ulex, Viola or Zinnia, particularly preferably selected from the group consisting of the plant genera Marigold, Tagetes erecta, Tagetes patula, Lycopersicon, Rosa, Calendula, Physalis, Medicago, Helianthus, Chrysanthemum, Aster, Tulipa, Narcissus, Petunia, Geranium, Tropaeolum or Adonis.

In the process according to the invention for the production of ketocarotenoids, a harvesting of the organisms and further preferably additionally an isolation of ketocarotenoids from the organisms follows the step of culturing the genetically modified organisms.

The harvesting of the organisms is carried out in a manner known per se corresponding to the respective organism. Microorganisms, such as bacteria, yeasts, algae or fungi or plant cells which are cultured by fermentation in liquid nutrient media, can be separated off, for example, by centrifuging, decanting or filtering. Plants are grown on the nutrient media in a manner known per se and harvested correspondingly.

The culturing of the genetically modified microorganisms is preferably carried out in the presence of oxygen at a culturing temperature of at least approximately 20° C., such as, for example, 20° C. to 40° C., and a pH of approximately 6 to 9. In the case of genetically modified microorganisms, the culturing of the microorganisms preferably takes place first in the presence of oxygen and in a complex medium, such as, for example, TB or LB medium, at a culturing temperature of approximately 20° C. or more, and a pH of approximately 6 to 9, until a sufficient cell density is achieved. In order to be able to control the oxidation reaction better, the use of an inducible promoter is preferred. The culturing is continued after induction of the ketolase expression in the presence of oxygen, e.g. for 12 hours to 3 days.

The isolation of the ketocarotenoids from the harvested biomass is carried out in a manner known per se, for example by extraction and, if appropriate, further chemical or physical purification processes, such as, for example, precipitation methods, crystallography, thermal separation processes, such as rectifying processes or physical separation processes, such as, for example, chromatography.

As mentioned below, the ketocarotenoids can be specifically produced in the genetically modified plants according to the invention, preferably in various plant tissues, such as, for example, seeds, leaves, fruit, flowers, in particular in flower leaves.

The isolation of ketocarotenoids from the harvested flower leaves is carried out in a manner known per se, for example by drying and subsequent extraction and, if appropriate, further chemical or physical purification processes, such as, for example, precipitation methods, crystallography, thermal separation processes, such as rectifying processes or physical separation processes such as, for example, chromatography. The isolation of ketocarotenoids from the flower leaves is carried out, for example, preferably by means of organic solvents such as acetone, hexane, ether or tert-methyl butyl ether.

Further processes of isolating ketocarotenoids, in particular from flower leaves, are described, for example, in Egger and Kleinig (Phytochemistry (1967) 6, 437-440) and Egger (Phytochemistry (1965) 4, 609-618).

Preferably, the ketocarotenoids are selected from the group consisting of astaxanthin, canthaxanthin, echinenone, 3-hydroxyechinenone, 3′-hydroxyechinenone, adonirubin and adonixanthin.

A particularly preferred ketocarotenoid is astaxanthin.

Depending on the organism used, the ketocarotenoids are obtained in free form or as fatty acid esters or as diglucosides.

In flower leaves of plants, the ketocarotenlids are obtained in the process according to the invention in the form of their mono- or diesters with fatty acids. Some fatty acids detected are, for example, myristic acid, palmitic acid, stearic acid, oleic acid, linolenic acid and lauric acid (Kamata and Simpson (1987) Comp. Biochem. Physiol. Vol. 86B(3), 587-591).

The production of the ketocarotenoids can take place in the whole plant or, in a preferred embodiment, specifically in plant tissues which comprise chromoplasts. Preferred plant tissues are, for example, roots, seeds, leaves, fruit, flowers and in particular nectaries and flower leaves, which are also called petals.

In a particularly preferred embodiment of the process according to the invention, genetically modified plants are used which have the highest expression rate of a ketolase in flowers.

Preferably, this is achieved by the gene expression of the ketolase taking place under the control of a flower-specific promoter. For example, for this the nucleic acids described above, as described in detail below, are inserted into a nucleic acid construct functionally linked to a flower-specific promoter in the plant.

In a further, particularly preferred embodiment of the process according to the invention, genetically modified plants are used which have the highest expression rate of a ketolase in fruit.

Preferably, this is achieved by the gene expression of the ketolase taking place under the control of a fruit-specific promoter. For example, for this the nucleic acids described above, as described in detail below, are inserted into a nucleic acid construct functionally linked to a fruit-specific promoter in the plant.

In a further, particularly preferred, embodiment of the process according to the invention, genetically modified plants are used which have the highest expression rate of a ketolase in seeds.

Preferably, this is achieved by the gene expression of the ketolase taking place under the control of a seed-specific promoter. For example, for this the nucleic acids described above, as described in detail below, are inserted into a nucleic acid construct functionally linked to a seed-specific promoter in the plant.

The targeting in the chromoplasts is carried out by a functionally linked plastidic transit peptide.

Below, by way of example, the production of genetically modified plants having increased or caused ketolase activity and increased or caused β-cyclase activity is described, the modified β-cyclase activity being caused by a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

The increasing of further activities, such as, for example, the hydroxylase activity, HMG-CoA reductase activity, (E)-4-hydroxy-3-methylbut-2-enyl diphosphate reductase activity, 1-deoxy-D-xylose-5-phosphate synthase activity, 1-deoxy-D-xylose-5-phosphate reductoisomerase activity, isopentenyl diphosphate Δ-isomerase activity, geranyl diphosphate synthase activity, farnesyl diphosphate synthase activity, geranylgeranyl diphosphate synthase activity, phytoene synthase activity, phytoene desaturase activity, zeta-carotene desaturase activity, crtISO activity, FtsZ activity and/or MinD activity can be carried out analogously using the corresponding effect genes.

In the combinations of genetic modifications, the transformations can be carried out individually or by means of multiple constructs.

The production of the transgenic plants is preferably carried out by transformation of the starting plants with a nucleic acid construct which comprises the nucleic acids described above encoding a ketolase and encoding a β-cyclase, which are functionally linked to one or more regulation signals which guarantee transcription and translation in plants, the nucleic acid encoding a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

Alternatively, the production of the transgenic plants is preferably carried out by transformation of the starting plants with two nucleic acid constructs. One nucleic acid construct comprises at least one nucleic acid described above, encoding a ketolase which is functionally linked to one or more regulation signals which guarantee transcription and translation in plants. The second nucleic acid construct comprises at least one nucleic acid described above, encoding a β-cyclase which is linked functionally to one or more regulation signals which guarantee transcription and translation in plants, the nucleic acid encoding a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

These nucleic acid constructs, in which the effect genes are linked functionally to one or more regulation signals which guarantee transcription and translation in plants, are also called expression cassettes below.

Preferably, the regulation signals comprise one or more promoters which guarantee transcription and translation in plants.

The expression cassettes comprise regulation signals, that is regulative nucleic acid sequences which control the expression of the effect genes in the host cell. According to a preferred embodiment, an expression cassette comprises upstream, i.e. at the 5′-end of the coding sequence, a promoter and downstream, i.e. at the 3′-end, a polyadenylation signal and, if appropriate, further regulatory elements which, with the coding sequence of the effect gene lying in between, are operatively linked to at least one of the genes described above. An operative linkage is understood as meaning the sequential arrangement of promoter, coding sequence, terminator and, if appropriate, further regulative elements in such a way that each of the regulative elements can fulfill its function in the expression of the coding sequence as intended.

Below, the preferred nucleic acid constructs, expression cassettes and vectors for plants and processes for the production of transgenic plants, and the transgenic plants themselves are described by way of example.

The sequences preferred, but not restricted thereto, for the operative linkage are targeting sequences for guaranteeing the subcellular localization in the apoplast, in the vacuoles, in plastids, in the mitochondrium, in the endoplasmatic reticulum (ER), in the cell nucleus, in elaioplasts or other compartments and translation enhancers such as the 5′ guide sequence from the tobacco mosaic virus (Gallie et al., Nucl. Acids Res. 15 (1987), 8693-8711).

As a promoter of the expression cassette, in principle any promoter is suitable which can control the expression of foreign genes in plants.

“Constitutive” promoter means those promoters which guarantee expression in numerous, preferably all, tissues over a relatively long period of time in the development of the plants, preferably at all times in the development of the plants.

Preferably, in particular a plant promoter or a promoter which originates from a plant virus is used. In particular, a preferred promoter is that of the 35S transcript of the CaMV cauliflower mosaic virus (Franck et al. (1980) Cell 21:285-294; Odell et al. (1985) Nature 313:810-812; Shewmaker et al. (1985) Virology 140:281-288; Gardner et al. (1986) Plant Mol Biol 6:221-228), the 19S CaMV promoter (U.S. Pat. No. 5,352,605; WO 84/02913; Benfey et al. (1989) EMBO J. 8:2195-2202), the triose phosphate translocator (TPT) promoter from Arabidopsis thaliana Acc. No. AB006698, base pair 53242 to 55281; the gene beginning from bp 55282 is annotated by “phosphate/triose phosphate translocator”, or the 34S promoter from figwort mosaic virus Acc. No. X16673, base pair 1 to 554.

A further suitable constitutive promoter is the pds promoter (Pecker et al. (1992) Proc. Natl. Acad. Sci USA 89: 4962-4966) or the “rubisco small subunit (SSU)” promoter (U.S. Pat. No. 4,962,028), the legumin B promoter (GenBank Acc. No. X03677), the promoter of the nopaline synthase from Agrobacterium, the TR double promoter, the OCS (octopine synthase) promoter from Agrobacterium, the ubiquitin promoter (Holtorf S et al. (1995) Plant Mol Biol 29:637-649), the ubiquitin 1 promoter (Christensen et al. (1992) Plant Mol Biol 18:675-689; Bruce et al. (1989) Proc Natl Acad Sci USA 86:9692-9696), the Smas promoter, the cinnamyl alcohol dehydrogenase promoter (U.S. Pat. No. 5,683,439), the promoters of vacuolar ATPase subunits or the promoter of a proline-rich protein from wheat (WO 91/13991), the Pnit promoter (Y07648.L, Hillebrand et al. (1998), Plant. Mol. Biol. 36, 89-99, Hillebrand et al. (1996), Gene, 170, 197-200) and further promoters of genes whose constitutive expression in plants is known to the person skilled in the art.

The expression cassettes can also comprise a chemically inducible promoter (overview article: Gatz et al. (1997) Annu Rev Plant Physiol Plant Mol Biol 48:89-108), by which the expression of the effect genes in the plants can be controlled at a certain point in time. Promoters of this type, such as, for example, the PRP1 promoter (Ward et al. (1993) Plant Mol Biol 22:361-366), a promoter inducible by salicylic acid (WO 95/19443), a promoter inducible by benzenesulfonamide (EP 0 388 186), a promoter inducible by tetracycline (Gatz et al. (1992) Plant J 2:397404), a promoter inducible by abscisic acid (EP 0 335 528) or a promoter inducible by ethanol or cyclohexanone (WO 93/21334) can likewise be used.

Further, promoters are preferred which are induced by biotic or abiotic stress, such as, for example, the pathogen-inducible promoter of the PRP1 gene (Ward et al. (1993) Plant Mol Biol 22:361-366), the heat-inducible hsp70 or hsp80 promoter from tomato (U.S. Pat. No. 5,187,267), the cold-inducible alpha-amylase promoter from the potato (WO 96/12814), the light-inducible PPDK promoter or the wounding-inducible pinII promoter (EP375091).

Pathogen-inducible promoters comprise those of genes which are induced as a result of a pathogen attack, such as, for example, genes of PR proteins, SAR proteins, b-1,3-glucanase, chitinase etc. (for example Redolfi et al. (1983) Neth J Plant Pathol 89:245-254; Uknes, et al. (1992) The Plant Cell 4:645-656; Van Loon (1985) Plant Mol Viral 4:111-116; Marineau et al. (1987) Plant Mol Biol 9:335-342; Matton et al. (1987) Molecular Plant-Microbe Interactions 2:325-342; Somssich et al. (1986) Proc Natl Acad Sci USA 83:2427-2430; Somssich et al. (1988) Mol Gen Genetics 2:93-98; Chen et al. (1996) Plant J 10:955-966; Zhang and Sing (1994) Proc Natl Acad Sci USA 91:2507-2511; Warner, et al. (1993) Plant J 3:191-201; Siebertz et al. (1989) Plant Cell 1:961-968 (1989).

Also comprised are wounding-inducible promoters such as that of the pinII gene (Ryan (1990) Ann Rev Phytopath 28:425-449; Duan et al. (1996) Nat Biotech 14:494-498), of the wun1 and wun2 gene (U.S. Pat. No. 5,428,148), of the win1 and win2 gene (Stanford et al. (1989) Mol Gen Genet 215:200-208), of the systemin gene (McGurl et al. (1992) Science 225:1570-1573), of the WIP1 gene (Rohmeier et al. (1993) Plant Mol Biol 22:783-792; Ekelkamp et al. (1993) FEBS Letters 323:73-76), of the MPI gene (Corderok et al. (1994) The Plant J 6(2):141-150) and the like.

Further suitable promoters are, for example, fruit ripening-specific promoters, such as, for example, the fruit ripening-specific promoter from tomato (WO 94/21794, EP 409 625). Development-dependent promoters partly include the tissue-specific promoters, since the formation of individual tissue naturally takes place in a development-dependent manner.

Furthermore, those promoters are in particular preferred which ensure expression in tissues or plant parts, in which, for example, the biosynthesis of ketocarotenoids or its precursors takes place. Preferred promoters are, for example, those with specificities for the anthers, ovaries, petals, sepals, flowers, leaves, stalks, seeds and roots and combinations thereof.

Bulb- or tuber-, storage root- or root-specific promoters are, for example, the patatin promoter class I (B33) or the promoter of the cathepsin D inhibitor from potato.

Leaf-specific promoters are, for example, the promoter of the cytosolic FBPase from potato (WO 97/05900), the SSU promoter (small subunit) of the rubisco (ribulose 1,5-bis-phosphate carboxylase) or the ST-LSI promoter from potato (Stockhaus et al. (1989) EMBO J. 8:2445-2451).

Flower-specific promoters are, for example, the phytoene synthase promoter (WO 92/16635) or the promoter of the P-rr gene (WO 98/22593), the AP3 promoter from Arabidopsis thaliana, the CHRC promoter (chromoplast-specific carotenoid-associated protein (CHRC) gene promoter from Cucumis sativus Acc. No. AF099501, base pair 1 to 1532), the EPSP_synthase promoter (5-enolpyruvyl shikimate-3-phosphate synthase gene promoter from Petunia hybrida, Acc. No. M37029, base pair 1 to 1788), the PDS promoter (phytoene desaturase gene promoter from Solanum lycopersicum, Acc. No. U46919, base pair 1 to 2078), the DFR-A promoter (dihydroflavonol 4 reductase gene A promoter from Petunia hybrida, Acc. No. X79723, base pair 32 to 1902) or the FBP1 promoter (floral binding protein 1 gene promoter from Petunia hybrida, Acc. No. L10115, base pair 52 to 1069).

Anther-specific promoters are, for example, the 5126 promoter (U.S. Pat. No. 5,689,049, U.S. Pat. No. 5,689,051), the glob-I promoter or the g-zein promoter.

Seed-specific promoters are, for example, the ACPO5 promoter (acyl-carrier protein gene, WO9218634), the promoters AtS1 and AtS3 from Arabidopsis (WO 9920775), the LeB4 promoter from Vicia faba (WO 9729200 and U.S. Pat. No. 6,403,371), the napin promoter from Brassica napus (U.S. Pat. No. 5,608,152; EP 255378; U.S. Pat. No. 5,420,034), the SBP promoter from Vicia faba (DE 9903432) or the corn promoters End1 and End2 (WO 0011177).

Further promoters suitable for expression in plants are described in Rogers et al. (1987) Meth in Enzymol 153:253-277; Schardl et al. (1987) Gene 61:1-11 and Berger et al. (1989) Proc Natl Acad Sci USA 86:8402-8406).

In the process according to the invention, constitutive, seed-specific, fruit-specific, flower-specific and in particular flower leaf-specific promoters are particularly preferred.

The production of an expression cassette preferably takes place by fusion of a suitable promoter with at least one of the effect genes described above, and preferably a nucleic acid inserted between promoter and nucleic acid sequence, which codes for a plastid-specific transit peptide, and a polyadenylation signal according to customary recombination and cloning techniques, such as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and also in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

The preferably inserted nucleic acids, encoding a plastidic transit peptide, guarantee localization in plastids and in particular in chromoplasts.

Expression cassettes can also be used whose nucleic acid sequence codes for an effect gene-product fusion protein, a part of the fusion protein being a transit peptide which controls the translocation of the polypeptide. Transit peptides specific for the chromoplasts are preferred, which after translocation of the effect genes in the chromoplasts are removed enzymatically from the effect gene product part.

In particular, the transit peptide is preferred which is derived from the plastidic Nicotiana tabacum transketolase or another transit peptide (e.g. the transit peptide of the small subunit of the rubisco (rbcS) or of the ferredoxin NADP oxidoreductase and also the isopentenyl pyrophosphate isomerase-2) or its functional equivalent.

Nucleic acid sequences of three cassettes of the plastid transit peptide of the plastidic transketolase of tobacco in three reading frames as KpnI/BamHI fragments having an ATG codon in the NcoI cleavage site are particularly preferred:

pTP09
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGGGATCC_BamHI
pTP10
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGCTGGATCC_BamHI
pTP11
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGGGGATCC_BamHI

Further examples of a plastidic transit peptide are the transit peptide of the plastidic isopentenyl pyrophosphate isomerase-2 (IPP-2) from Arabisopsis thaliana and the transit peptide of the small subunit of ribulose bisphosphate carboxylase (rbcS) from pea (Guerineau, F, Woolston, S, Brooks, L, Mullineaux, P (1988) An expression cassette for targeting foreign proteins into the chloroplasts. Nucl. Acids Res. 16: 11380).

The nucleic acids according to the invention can be prepared synthetically or obtained naturally or comprise a mixture of synthetic and natural nucleic acid constituents, and consist of various heterologous gene sections of various organisms.

As described above, synthetic nucleotide sequences with codons which are preferably from plants are preferred. These preferred codons from plants can be identified from codons with the highest protein frequency, which are expressed in the most interesting plant species.

In the preparation of an expression cassette, various DNA fragments can be manipulated in order to obtain a nucleotide sequence which expediently reads in the correct direction and which is equipped with a correct reading frame. For the connection of the DNA fragments to one another, adapters or linkers can be attached to the fragments.

Expediently, the promoter and the terminator regions can be provided in the transcription direction with a linker or polylinker which comprises one or more restriction sites for the insertion of this sequence. As a rule, the linker has 1 to 10, usually 1 to 8, preferably 2 to 6, restriction sites. In general, the linker has, within the regulatory regions, a size of less than 100 bp, often less than 60 bp, but at least 5 bp. The promoter can be either native or homologous, or foreign or heterologous to the host plant. The expression cassette preferably comprises in the 5′-3′ transcription direction the promoter, a coding nucleic acid sequence or a nucleic acid construct and a region for transcriptional termination. Various termination regions are mutually exchangeable at will.

Examples of a terminator are the 35S terminator (Guerineau et al. (1988) Nucl Acids Res. 16: 11380), the nos terminator (Depicker A, Stachel S, Dhaese P, Zambryski P, Goodman H M. Nopaline synthase: transcript mapping and DNA sequence. J Mol Appl Genet. 1982; 1 (6):561-73) or the ocs terminator (Gielen, J, de Beuckeleer, M, Seurinck, J, Debroek, H, de Greve, H, Lemmers, M, van Montagu, M, Schell, J (1984) The complete sequence of the TL-DNA of the Agrobacterium tumefaciens plasmid pTiAch5. EMBO J. 3: 835-846).

Furthermore, manipulations which make available suitable restriction cleavage sites or remove the superfluous DNA or restriction cleavage sites can be employed. Where insertions, deletions or substitutions such as, for example, transitions and transversions are possible, in vitro mutagenesis, “primer repair”, restriction or ligation can be used.

With suitable manipulations, such as, for example, restriction, “chewing-back” or filling of overhangs for “blunt ends”, complementary ends of the fragments can be made available for ligation.

Preferred polyadenylation signals are plant polyadenylation signals, preferably those which essentially correspond to T-DNA polyadenylation signals from Agrobacterium tumefaciens, in particular of the gene 3 of the T-DNA (octopine synthase) of the Ti plasmid pTiACH5 (Gielen et al., EMBO J. 3 (1984), 835 ff) or functional equivalents.

The transfer of foreign genes to the genome of a plant is called transformation.

To this end, methods known per se for the transformation and regeneration of plants from plant tissues or plant cells can be utilized for transient or stable transformation.

Suitable methods for the transformation of plants are protoplast transformation by polyethylene glycol-induced DNA uptake, the biolistic process using the gene gun—the “particle bombardment” method, electroporation, the incubation of dry embryos in DNA-containing solution, microinjection and gene transfer, described above, mediated by Agrobacterium. The processes mentioned are described, for example, in B. Jenes et al., Techniques for Gene Transfer, in: Transgenic Plants, Vol. 1, Engineering and Utilization, edited by S. D. Kung and R. Wu, Academic Press (1993), 128-143 and in Potrykus, Annu. Rev. Plant Physiol. Plant Molec. Biol. 42 (1991), 205-225).

Preferably, the construct to be expressed is cloned in a vector which is suitable for transforming Agrobacterium tumefaciens, for example pBin19 (Bevan et al., Nucl. Acids Res. 12 (1984), 8711) or particularly preferably pSUN2, pSUN3, pSUN4 or pSUN5 (WO 02/00900).

Agrobacteria transformed using an expression plasmid can be used in a known manner for the transformation of plants, e.g. by bathing wounded leaves or pieces of leaf in an Agrobacteria solution and subsequently culturing in suitable media.

For the preferred production of genetically modified plants, also called transgenic plants below, the fused expression cassette is cloned in a vector, for example pBin19 or in particular pSUN5 and pSUN3, which is suitable to be transformed in Agrobacterium tumefaciens. Agrobacteria transformed using such a vector can then be used in a known manner for the transformation of plants, in particular of crop plants, by, for example, bathing wounded leaves or pieces of leaf in an Agrobacteria solution and subsequently culturing in suitable media.

The transformation of plants by Agrobacteria is known, inter alia, from F. F. White, Vectors for Gene Transfer in Higher Plants; in Transgenic Plants, Vol. 1, Engineering and Utilization, edited by S. D. Kung and R. Wu, Academic Press, 1993, pp. 15-38. From the transformed cells of the wounded leaves or pieces of leaf, transgenic plants can be regenerated in a known manner which comprise one or more genes integrated into the expression cassette.

For the transformation of a host plant using one or more effect genes according to the invention, an expression cassette is incorporated into a recombinant vector as an insertion whose vector DNA comprises additional functional regulation signals, for example sequences for replication or integration. Suitable vectors are described, inter alia, in “Methods in Plant Molecular Biology and Biotechnology” (CRC Press), Chap. 6/7, pp. 71-119 (1993).

Using the recombination and cloning techniques cited above, the expression cassettes can be cloned in suitable vectors which make possible their proliferation, for example in E. coli. Suitable cloning vectors are, inter alia, pJIT117 (Guerineau et al. (1988) Nucl. Acids Res. 16:11380), pBR332, pUC series, M13 mp series and pACYC184. Particularly suitable are binary vectors, which can replicate both in E. coli and in Agrobacteria.

Below, by way of example, the production of genetically modified microorganisms according to the invention having increased or caused ketolase activity and increased or caused β-cyclase activity is described in greater detail, the modified β-cyclase activity being caused by a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

The increasing of further activities, such as, for example, the hydroxylase activity, HMG-CoA reductase activity, (E)-4-hydroxy-3-methylbut-2-enyl diphosphate reductase activity, 1-deoxy-D-xylose-5-phosphate synthase activity, 1-deoxy-D-xylose-5-phosphate reductoisomerase activity, isopentenyl diphosphate Δ-isomerase activity, geranyl diphosphate synthase activity, farnesyl diphosphate synthase activity, geranylgeranyl diphosphate synthase activity, phytoene synthase activity, phytoene desaturase activity, zeta-carotene desaturase activity, crtISO activity, FtsZ activity and/or MinD activity can be carried out analogously using the corresponding effect genes.

The nucleic acids described above, encoding a ketolase, β-hydroxylase or β-cyclase, and the nucleic acids encoding an HMG-CoA reductase, nucleic acids encoding an (E)-4-hydroxy-3-methylbut-2-enyl diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl diphosphate Δ-isomerase, nucleic acids encoding a geranyl diphosphate synthase, nucleic acids encoding a farnesyl diphosphate synthase, nucleic acids encoding a geranylgeranyl diphosphate synthase, nucleic acids encoding a phytoene synthase, nucleic acids encoding a phytoene desaturase, nucleic acids encoding a zeta-carotene desaturase, nucleic acids encoding a crtISO protein, nucleic acids encoding an FtsZ protein and/or nucleic acids encoding a MinD protein are preferably inserted in expression constructs comprising, under the genetic control of regulative nucleic acid sequences, a nucleic acid sequence coding for an enzyme according to the invention; and vectors comprising at least one of these expression constructs.

Preferably, such constructs according to the invention comprise, 5′-upstream from the respective coding sequence, a promoter and 3′-downstream a terminator sequence and, if appropriate, further customary regulative elements, namely in each case operatively linked with the effect gene. An “operative linkage” is understood as meaning the sequential arrangement of promoter, coding sequence (effect gene), terminator and, if appropriate, further regulative elements in such a way that each of the regulative elements can fulfill its function in the expression of the coding sequence as intended.

Examples of operatively linkable sequences are targeting sequences and translation enhancers, enhancers, polyadenylation signals and the like. Further regulative elements comprise selectable markers, amplification signals, replication origins and the like.

In addition to the artificial regulation sequences, the natural regulation sequence can still be present before the actual effect gene. By means of genetic modification, this natural regulation can, if appropriate, be switched off and the expression of the genes increased or decreased. The gene construct can, however, also be of simpler construction, that is no additional regulation signals are inserted before the structural gene, and the natural promoter with its regulation is not removed. Instead of this, the natural regulation sequence is mutated such that regulation no longer takes place and the gene expression is increased or decreased. The nucleic acid sequences can be comprised in one or more copies in the gene construct.

Examples of utilizable promoters in microorganisms are: cos-, tac-, trp-, tet-, trp-tet-, lpp-, lac-, lpp-lac-, laclq-, T7-, T5-, T3-, gal-, trc-, ara-, SP6-, lambda-PR- or in the lambda-PL promoter, which are advantageously used in gram-negative bacteria; and the gram-positive promoters amy and SPO2 or the yeast promoters ADC1, MFa, AC, P-60, CYC1, GAPDH. The use of inducible promoters is particularly preferred, such as, for example, light- and in particular temperature-inducible promoters, such as the PᵣPₗ promoter.

In principle, all natural promoters with their regulation sequences can be used. Moreover, synthetic promoters can also advantageously be used.

Said regulatory sequences should make possible the selective expression of the nucleic acid sequences and the protein expression. This can mean, for example, depending on the host organism, that the gene is expressed or overexpressed only after induction, or that it is immediately expressed and/or overexpressed.

The regulatory sequences or factors can in this case preferably positively influence the expression and thereby increase or decrease it. Thus an enhancement of the regulatory elements can advantageously take place at the transcription level by using strong transcription signals such as promoters and/or “enhancers”. In addition, however, an enhancement of the translation is also possible by, for example, improving the stability of the mRNA.

The production of an expression cassette is carried out by fusion of a suitable promoter with the nucleic acid sequences described above, encoding a ketolase, β-hydroxylase, β-cyclase, HMG-CoA reductase, (E)-4-hydroxy-3-methylbut-2-enyl diphosphate reductase, 1-deoxy-D-xylose-5-phosphate synthase, 1-deoxy-D-xylose-5-phosphate reductoisomerase, isopentenyl diphosphate Δ-isomerase, geranyl diphosphate synthase, farnesyl diphosphate synthase, geranylgeranyl diphosphate synthase, phytoene synthase, phytoene desaturase, zeta-carotene desaturase, crtISO protein, FtsZ protein and/or an MinD protein and a terminator or polyadenylation signal. To this end, customary recombination and cloning techniques are used, such as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and also in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

The recombinant nucleic acid construct or gene construct is, for expression in a suitable host organism, advantageously inserted into a host-specific vector, which makes possible an optimum expression of the genes in the host. Vectors are well known to the person skilled in the art and can be inferred, for example, from “Cloning Vectors” (Pouwels P. H. et al., Ed, Elsevier, Amsterdam-New York-Oxford, 1985). Vectors, apart from plasmids, are also understood as meaning all other vectors known to the person skilled in the art, such as, for example, phages, viruses, such as SV40, CMV, baculovirus and adenovirus, transposons, IS elements, phasmids, cosmids, and linear or circular DNA. These vectors can be replicated autonomically in the host organism or replicated chromosomally.

Examples of suitable expression vectors which can be mentioned are:

Customary fusion expression vectors, such as pGEX (Pharmacia Biotech Inc; Smith, D. B. and Johnson, K. S. (1988) Gene 67:31-40), pMAL (New England Biolabs, Beverly, Mass.) and pRIT 5 (Pharmacia, Piscataway, N.J.), in which glutathione S-transferase (GST), maltose E-binding protein or protein A is fused to the recombinant target protein.

Non-fusion protein expression vectors such as pTrc (Amann et al., (1988) Gene 69:301-315) and pET 11d (Studier et al. Gene Expression Technology: Methods in Enzymology 185, Academic Press, San Diego, Calif. (1990) 60-89) or pBluescript and pUC vectors.

Yeast expression vectors for expression in the yeast S. cerevisiae, such as pYepSec1 (Baldari et al., (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, Calif.).

Vectors and processes for the construction of vectors which are suitable for use in other fungi, such as filamentous fungi, comprise those which are described in detail in: van den Hondel, C. A. M. J. J. & Punt, P. J. (1991) “Gene transfer systems and vector development for filamentous fungi, in: Applied Molecular Genetics of Fungi, J. F. Peberdy et al., Ed., S. 1-28, Cambridge University Press: Cambridge.

Baculovirus vectors, which are available for the expression of proteins in cultured insect cells (e.g. Sf9 cells), comprise the pAc series (Smith et al., (1983) Mol. Cell Biol. 3:2156-2165) and the pVL series (Lucklow and Summers (1989) Virology 170:31-39).

Further suitable expression systems for prokaryotic and eukaryotic cells are described in chapters 16 and 17 of Sambrook, J., Fritsch, E. F. and Maniatis, T., Molecular cloning: A Laboratory Manual, 2nd edition, Cold Spring Harbor Laboratory, Cold Spring Harbor Laboratory Press, Cold Spring Harbor, N.Y., 1989.

With the aid of the expression constructs or vectors according to the invention, genetically modified organisms can be prepared which are transformed, for example, using at least one vector according to the invention.

Advantageously, the recombinant constructs according to the invention described above are inserted into a suitable host system and expressed. In this connection, familiar cloning and transfection methods preferably known to the person skilled in the art, such as, for example, co-precipitation, protoplast fusion, electroporation, retroviral transfection and the like, in order to express said nucleic acids in the respective expression system. Suitable systems are described, for example, in Current Protocols in Molecular Biology, F. Ausubel et al., Ed., Wiley Interscience, New York 1997.

The selection of successfully transformed organisms can be carried out by marker genes which are likewise comprised in the vector or in the expression cassette. Examples of such marker genes are genes for antibiotic resistance and for enzymes which catalyze a coloring reaction, which causes a staining of the transformed cell. These can then be selected by means of automatic cell sorting.

Microorganisms transformed successfully using a vector, which carry an appropriate antibiotic resistance gene (e.g. G418 or hygromycin), can be selected by means of appropriate antibiotic-comprising media or nutrient media. Marker proteins which are presented on the cell surface can be utilized for selection by means of affinity chromatography.

The combination of the host organisms and the vectors suitable for the organisms, such as plasmids, viruses or phages, such as, for example, plasmids having the RNA polymerase/promoter system, the phages 8 or other temperent phages or transposons and/or other advantageous regulatory sequences, forms an expression system.

The invention further relates to the genetically modified, nonhuman organisms, where the genetic modification

A for the case where the wild-type organism already has a ketolase activity, increases the activity of a ketolase compared to the wild-type and

B for the case where the wild-type organism has no ketolase activity, causes the activity of a ketolase compared to the wild-type,

and where the genetic modification

C for the case where the wild-type organism already has a β-cyclase activity, increases the activity of a β-cyclase compared to the wild-type and

D for the case where the wild-type organism has no β-cyclase activity, causes the activity of a β-cyclase compared to the wild-type

and the β-cyclase activity increased according to C or caused according to D is caused by a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

As explained above, the increasing (according to A) or causing (according to B) of the ketolase activity compared to the wild-type preferably takes place by the increasing of the gene expression of a nucleic acid encoding a ketolase.

In a further preferred embodiment, the increasing of the gene expression of a nucleic acid encoding a ketolase is carried out by inserting nucleic acids which encode ketolases into the organism.

In the transgenic organisms according to the invention, in this embodiment compared to the wild-type at least one further ketolase gene is thus present. In this embodiment, the genetically modified organism according to the invention preferably has at least one exogenous (=heterologous) nucleic acid encoding a ketolase, or has at least two endogenous nucleic acids encoding a ketolase: To this end, in principle any ketolase gene, that is any nucleic acids which encodes a ketolase, can be used.

Preferred nucleic acids encoding a ketolase are described above in the process according to the invention.

Preferably, the increasing or causing of the β-cyclase activity, as described above, is carried out by increasing the gene expression compared to the wild-type of nucleic acids encoding a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

In a preferred embodiment, the increasing of the gene expression of a nucleic acid encoding a β-cyclase by insertion into the organism of at least one nucleic acid encoding a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2.

In the transgenic organisms according to the invention, in this embodiment at least one further β-cyclase gene is thus present compared to the wild-type. In this embodiment, the genetically modified organism according to the invention preferably has at least one exogenous (=heterologous) nucleic acid encoding a β-cyclase, or at least two endogenous nucleic acids encoding a β-cyclase.

To this end, in principle any β-cyclase gene, that is any nucleic acid which encodes a β-cyclase comprising the amino acid sequence SEQ. ID. NO. 2 or a sequence derived from this sequence by substitution, insertion or deletion of amino acids, which has an identity of at least 70% at the amino acid level with the sequence SEQ. ID. NO. 2, can be used.

Preferred β-cyclase genes are described above.

Particularly preferred genetically modified organisms, as mentioned above, additionally have an increased or caused hydroxlase activity compared to the wild-type organism. Further preferred embodiments are described above in the process according to the invention.

Further, particularly preferred, genetically modified nonhuman organisms, as mentioned above, additionally have, compared to the wild-type, at least one further increased activity, selected from the group consisting of HMG-CoA reductase activity, (E)-4-hydroxy-3-methylbut-2-enyl diphosphate reductase activity, 1-deoxy-D-xylose-5-phosphate synthase activity, 1-deoxy-D-xylose-5-phosphate reductoisomerase activity, isopentenyl diphosphate Δ-isomerase activity, geranyl diphosphate synthase activity, farnesyl diphosphate synthase activity, geranylgeranyl diphosphate synthase activity, phytoene synthase activity, phytoene desaturase activity, zeta-carotene desaturase activity, crtISO activity, FtsZ activity and MinD activity. Further preferred embodiments are described above in the process according to the invention.

Organisms are understood according to the invention preferably as meaning organisms which, as the wild-type or starting organisms, naturally or by genetic complementation and/or reregulation of the metabolic pathways are in the position to produce carotenoids, in particular β-carotene and/or zeaxanthin and/or neoxanthin and/or violaxanthin and/or lutein.

Further preferred organisms, as the wild-type or starting organisms, already have a hydroxylase activity and are thus in the position, as wild-type or starting organisms, to produce zeaxanthin.

Preferred organisms are plants or microorganisms such as, for example, bacteria, yeasts, algae or fungi.

The bacteria used can be either bacteria which, on account of the insertion of genes of the carotenoid biosynthesis of a carotenoid-producing organism, are in the position to synthesize xanthophylls, such as, for example, bacteria of the genus Escherichia, which, for example, comprise crt genes from Erwinia, also bacteria which by themselves are in the position to synthesise xanthophylls, such as, for example, bacteria of the genus Erwinia, Agrobacterium, Flavobacterium, Alcaligenes, Paracoccus, Nostoc or cyanobacteria of the genus Synechocystis.

Preferred bacteria are Escherichia coli, Erwinia herbicola, Erwinia uredovora, Agrobacterium aurantiacum, Alcaligenes sp. PC-1, Flavobacterium sp. strain R1534, the cyanobacterium Synechocystis sp. PCC6803, Paracoccus marcusii or Paracoccus caroteneifaciens.

Preferred yeasts are Candida, Saccharomyces, Hansenula, Pichia or Phaffia. Particularly preferred yeasts are Xanthophyllomyces dendrorhous or Phaffia rhodozyma.

Preferred fungi are Aspergillus, Trichoderma, Ashbya, Neurospora, Blakeslea, in particular Blakeslea trispora, Phycomyces, Fusarium or further fungi described in Indian Chem. Engr. Section B. Vol. 37, No. 1, 2 (1995) on page 15, Table 6.

Preferred algae are green algae, such as, for example, algae of the genus Haematococcus, Phaedactylum tricornatum, Volvox or Dunaliella. Particularly preferred algae are Haematococcus puvialis or Dunaliella bardawil.

Further utilizable microorganisms and their production for carrying out the process according to the invention are known, for example, from DE-A-199 16 140, to which reference is hereby made.

Particularly preferred plants are plants selected from the families Amaranthaceae, Amaryllidaceae, Apocynaceae, Asteraceae, Balsaminaceae, Begoniaceae, Berberidaceae, Brassicaceae, Cannabaceae, Caprifoliaceae, Caryophyllaceae, Chenopodiaceae, Compositae, Cucurbitaceae, Cruciferae, Euphorbiaceae, Fabaceae, Gentianaceae, Geraniaceae, Graminae, Illiaceae, Labiatae, Lamiaceae, Leguminosae, Liliaceae, Linaceae, Lobeliaceae, Malvaceae, Oleaceae, Orchidaceae, Papaveraceae, Plumbaginaceae, Poaceae, Polemoniaceae, Primulaceae, Ranunculaceae, Rosaceae, Rubiaceae, Scrophulariaceae, Solanaceae, Tropaeolaceae, Umbelliferae, Verbanaceae, Vitaceae and Violaceae.

Very particularly preferred plants are selected from the group consisting of the plant genera Marigold, Tagetes errecta, Tagetes patula, Acacia, Aconitum, Adonis, Arnica, Aquilegia, Aster, Astragalus, Bignonia, Calendula, Caltha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hisbiscus, Heliopsis, Hypericum, Hypochoeris, Impatiens, Iris, Jacaranda, Kerria, Laburnum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Sorbus, Spartium, Tecoma, Torenia, Tragopogon, Trollius, Tropaeolum, Tulipa, Tussilago, Ulex, Viola or Zinnia, particularly preferably selected from the group consisting of the plant genera Marigold, Tagetes erecta, Tagetes patula, Lycopersicon, Rosa, Calendula, Physalis, Medicago, Helianthus, Chrysanthemum, Aster, Tulipa, Narcissus, Petunia, Geranium, Tropaeolum or Adonis.

Very particularly preferred genetically modified plants are selected from the plant genera Marigold, Tagetes erecta, Tagetes patula, Adonis, Lycopersicon, Rosa, Calendula, Physalis, Medicago, Helianthus, Chrysanthemum, Aster, Tulipa, Narcissus, Petunia, Geranium or Tropaeolum, the genetically modified plant comprising at least one transgenic nucleic acid encoding an ketolase.

The transgenic plants, their reproductive material, and their plant cells, tissue or parts, in particular their fruit, seeds, flowers and flower leaves are a further subject of the present invention.

The genetically modified plants can, as described above, be used for the production of ketocarotenoids, in particular astaxanthin.

Genetically modified organisms according to the invention consumable by humans and animals, in particular plants or plant parts, such as, in particular, flower leaves having an increased content of ketocarotenoids, in particular astaxanthin, can also be used, for example, directly or after processing known per se as foods or feeds or as feed and food supplements.

Furthermore, the genetically modified organisms can be used for the production of ketocarotenoid-containing extracts of the organisms and/or for the production of feed and food supplements.

The genetically modified organisms have, in comparison with the wild-type, an increased content of ketocarotenoids.

An increased content of ketocarotenoids is as a rule understood as meaning an increased content of total ketocarotenoid.

An increased content of ketocarotenoids is, however, also understood in particular as meaning a modified content of the preferred ketocarotenoids, without the total carotenoid content inevitably having to be increased.

In a particularly preferred embodiment, the genetically modified plants according to the invention have an increased content of astaxanthin in comparison with the wild-type.

An increased content is in this case also understood as meaning a caused content of ketocarotenoids, or astaxanthin.

The invention is illustrated by the examples which now follow, but is not restricted to these:

General Experimental Conditions:

Sequence Analysis of Recombinant DNA

The sequencing of recombinant DNA molecules was carried out using a laser fluorescence DNA sequencer from Licor (marketed by MWG Biotech, Ebersbach) according to the method of Sanger (Sanger et al., Proc. Natl. Acad. Sci. USA 74 (1977), 5463-5467).

### EXAMPLE 1

Amplification of a DNA which Encodes the Entire Primary Sequence of the NOST Ketolase from Nostoc sp. PCC 7120

The DNA which codes for the NOST ketolase from Nostoc sp. PCC 7120 was amplified by means of PCR from Nostoc sp. PCC 7120 (strain of the “Pasteur Culture Collection of Cyanobacterium”).

For the preparation of genomic DNA from a suspension culture of Nostoc sp. PCC 7120 which had been grown for 1 week with continuous illumination and constant shaking (150 rpm) at 25° C. in BG 11 medium (1.5 g/l of NaNO3, 0.04 g/l of K2PO4×3H₂O, 0.075 g/l of MgSO4×H₂O, 0.036 g/l of CaCl2×2H₂O, 0.006 g/l of citric acid, 0.006 g/l of ferric ammonium citrate, 0.001 g/l of EDTA disodium magnesium, 0.04 g/l of Na2CO3, 1 ml of trace metal mix “A5+Co” (2.86 g/l H3BO3, 1.81 g/l of MnCl2×4H2o, 0.222 g/l of ZnSO4×7H2o, 0.39 g/l of NaMoO4×2H2O, 0.079 g/l of CuSO4×5H2O, 0.0494 g/l of Co(NO3)2×6H₂O)), the cells were harvested by centrifugation, frozen in liquid nitrogen and pulverized in a mortar.

Protocol for DNA Isolation from Nostoc PCC7120:

The bacterial cells from a 10 ml liquid culture were pelleted by centrifugation at 8000 rpm for 10 minutes. Subsequently, the bacterial cells were pulverized and ground in liquid nitrogen using a mortar. The cell material was resuspended in 1 ml 10 mM Tris HCl (pH 7.5) and transferred to an Eppendorf reaction vessel (2 ml volume). After addition of 100 μl of proteinase K (conzentration: 20 mg/ml), the cell suspension was incubated for 3 hours at 37° C. Subsequently, the suspension was extracted using 500 μl of phenol. After centrifugation at 13 000 rpm for 5 minutes, the upper, aqueous phase was transferred to a new 2 ml Eppendorf reaction vessel. The extraction with phenol was repeated 3 times. The DNA was precipitated by addition of a 1/10 volume of 3 M sodium acetate (pH 5.2) and 0.6 volume of isopropanol and subsequently washed with 70% ethanol. The DNA pellet was dried at room temperature, taken up in 25 μl of water and dissolved at 65° C. with heating.

The nucleic acid encoding a ketolase from Nostoc PCC 7120 was amplified by means of “polymerase chain reaction” (PCR) from Nostoc sp. PCC 7120 using a sense-specific primer (NOSTF, SEQ ID No. 79) and an antisense-specific primer (NOSTG SEQ ID No. 80).

The PCR conditions used were as below:

The PCR for the amplification of the DNA which codes for a ketolase protein consisting of the entire primary sequence was carried out in a 50 ul reaction batch, in which was comprised:
1 ul of of a Nostoc sp. PCC 7120 DNA (prepared as described above)
0.25 mM dNTPs
0.2 mM NOSTF (SEQ ID No. 79)
0.2 mM NOSTG (SEQ ID No. 80)
5 ul of 10×PCR buffer (TAKARA)
0.25 ul of R Taq polymerase (TAKARA)
25.8 ul of dist. water.

The PCR was carried out under the following cycle conditions:
1×94° C. 2 minutes
35×94° C. 1 minute
55° C. 1 minutes
72° C. 3 minutes
1×72° C. 10 minutes

The PCR amplification with SEQ ID No. 79 and SEQ ID No. 80 resulted in an 805 bp fragment, which codes for a protein consisting of the entire primary sequence (SEQ ID No. 81). Using standard methods, the amplificate was cloned in the PCR cloning vector pGEM-T (Promega) and the clone pNOSTF-G was obtained.

Sequencing of the clone pNOSTF-G using the M13F and the M13R primer confirmed a sequence which is identical with the DNA sequence of 88,886-89,662 of the database entry AP003592. This nucleotide sequence was reproduced in an independent amplification experiment and thus represents the nucleotide sequence in the Nostoc sp. PCC 7120 used.

This clone pNOSTF-G was therefore used for the cloning in the expression vector pJIT117 (Guerineau et al. 1988, Nucl. Acids Res. 16: 11380). The cloning was carried out by isolation of the 799 Bp SphI fragment from pNOSTF-G and ligation in the SphI-cleaved vector pJIT117. The clone which comprises the ketolase of Nostoc sp. PCC 7120 in the correct orientation as an N-terminal translational fusion with the rbcS transit peptide is called pJNOST.

### EXAMPLE 2

Construction of the Plasmid pMCL-CrtYIBZ/idi/gps for the Synthesis of Zeaxanthin in E. coli

The construction of pMCL-CrtYIBZ/idi/gps was carried out in three steps via the intermediate stages pMCL-CrtYIBZ and pMCL-CrtYIBZ/idi. As a vector, the plasmid pMCL200 compatible with high-copy number vectors was used (Nakano, Y., Yoshida, Y., Yamashita, Y. and Koga, T.; Construction of a series of pACYC-derived plasmid vectors; Gene 162 (1995), 157-158).

### Example 2.1

### Construction of pMCL-CrtYIBZ

The biosynthesis genes crtY, crtB, crtI and crtZ originate from the bacterium Erwinia uredovora and were amplified by means of PCR. Genomic DNA from Erwinia uredovora (DSM 30080) was prepared by the German Collection of Microorganisms and Cell Cultures (DSMZ, Brunswick) in a service unit. The PCR reaction was carried out according to the details of the manufacturer (Roche, Long Template PCR: Procedure for amplification of 5-20 kb targets with the expand long template PCR system). The PCR conditions for the amplification of the biosynthesis cluster of Erwinia uredovora were as below:

Master Mix 1:

1.75 ul of dNTPs (final concentration 350 μM)
0.3 μM primer Crt1 (SEQ ID No. 82)
0.3 μM primer Crt2 (SEQ ID No. 83)
250-500 ng of genomic DNA of DSM 30080
   dist. water up to a total volume of 50 μl
   Master Mix 2:
5 ul of 10×PCR buffer 1 (final concentration 1×, comprising 1.75 mM Mg2+)
10×PCR buffer 2 (final concentration 1×, comprising 2.25 mM Mg2+)
10×PCR buffer 3 (final concentration 1×, comprising 2.25 mM Mg2+)
0.75 ul of Expand Long Template Enzyme Mix (final concentration 2.6 units)
   dist. water up to a total volume of 50 μl

The two batches “Master Mix 1” and “Master Mix 2” were pipetted together. The PCR was carried out in a total volume of 50 ul under the following cycle conditions:

1×94° C. 2 minutes

30×94° C. 30 seconds

58° C. 1 minute
68° C. 4 minutes
   1×72° C. 10 minutes

The PCR amplification with SEQ ID No. 82 and SEQ ID No. 83 resulted in a fragment (SEQ ID NO: 84) which codes for the genes CrtY (protein: SEQ ID NO: 85), CrtI (protein: SEQ ID NO: 86), crtB (protein: SEQ ID NO: 87) and CrtZ (iDNA). Using standard methods, the amplificate was cloned in the PCR cloning vector pCR2.1 (Invitrogen) and the clone pCR2.1-CrtYIBZ was obtained.

The plasmid pCR2.1-CrtYIBZ was cleaved by SalI and HindIII, the resulting SalI/HindIII fragments was isolated and transferred by ligation to the SalI/HindIII-cleaved vector pMCL200. The SalI/HindIII fragment from pCR2.1-CrtYIBZ cloned in pMCL 200 is 4624 bp long, codes for the genes CrtY, CrtI, crtB and CrtZ and corresponds to the sequence of position 2295 to 6918 in D90087 (SEQ ID No. 84). The gene CrtZ is transcribed against the reading direction of the genes CrtY, CrtI and CrtB by means of its endogenous promoter. The resulting clone is called pMCL-CrtYIBZ.

### Example 2.2

### Construction of pMCL-CrtYIBZ/idi

The gene idi (isopentenyl phosphate isomerase; IPP isomerase) was amplified from E. coli by means of PCR. The nucleic acid encoding the entire idi gene with idi promoter and ribosome binding site was amplified from E. coli by means of “polymerase chain reaction” (PCR) using a sense-specific primer (5′-idi SEQ ID No. 88) and an antisense-specific primer (3′-idi SEQ ID No. 89).

The PCR conditions were as follows:

The PCR for the amplification of the DNA was carried out in a 50 μl reaction batch, in which was comprised:
1 ul of an E. coli TOP10 suspension
0.25 mM dNTPs
0.2 mM 5′-idi (SEQ ID No. 88)
0.2 mM 3′-idi (SEQ ID No. 89)
5 ul of 10×PCR buffer (TAKARA)
0.25 ul of R Taq polymerase (TAKARA)
28.8 ul of dist. water

The PCR was carried out under the following cycle conditions:

1×94° C. 2 minutes

20×94° C. 1 minute

62° C. 1 minute
72° C. 1 minute
   1×72° C. 10 minutes

The PCR amplification with SEQ ID No. 88 and SEQ ID No. 89 resulted in a 679 bp fragment which codes for a protein consisting of the entire primary sequence (SEQ ID No. 90). Using standard methods, the amplificate was cloned in the PCR cloning vector pCR2.1 (Invitrogen) and the clone pCR2.1-idi was obtained.

Sequencing of the clone pCR2.1-idi confirmed a sequence which did not differ from the published sequence AE000372 in position 8774 to position 9440. This region comprises the promoter region, the potential ribosome binding site and the entire “open reading frame” for the IPP isomerase. The fragment cloned in pCR2.1-idi has, owing to the insertion of an XhoI cleavage site at the 5′-end and a SalI-cleavage site at the 3′-end of the idi gene, a total length of 679 bp.

This clone was therefore used for the cloning of the idi gene in the vector pMCL-CrtYIBZ. The cloning was carried out by isolation of the XhoI/SalI fragment from pCR2.1-idi and ligation in the XhoI/SalI-cleaved vector pMCL-CrtYIBZ. The resulting clone is called pMCL-CrtYIBZ/idi.

### Example 2.3

### Construction of pMCL-CrtYIBZ/idi/gps

The gene gps (geranylgeranyl pyrophosphate synthase; GGPP synthase) was amplified from Archaeoglobus fulgidus by means of PCR. The nucleic acid encoding gps from Archaeoglobus fulgidus was amplified by means of “polymerase chain reaction” (PCR) using a sense-specific primer (5′-gps SEQ ID No. 92) and an antisense-specific primer (3′-gps SEQ ID No. 93).

The DNA from Archaeoglobus fulgidus was prepared by the German Collection of Microorganisms and Cell Cultures (DSMZ, Brunswick) in a service unit. The PCR conditions were as follows:

The PCR for the amplification of the DNA which codes for a GGPP synthase protein consisting of the entire primary sequence was carried out in a 50 μl reaction batch, in which was comprised:
1 ul of an Archaeoglobus fulgidus DNA
0.25 mM dNTPs
0.2 mM 5′-gps (SEQ ID No. 92)
0.2 mM 3′-gps (SEQ ID No. 93)
5 ul of 10×PCR buffer (TAKARA)
0.25 ul of R Taq polymerase (TAKARA)
28.8 ul of dist. water

The PCR was carried out under the following cycle conditions:

1×94° C. 2 minutes

20×94° C. 1 minute

56° C. 1 minute
72° C. 1 minute
   1×72° C. 10 minutes

The DNA fragment amplified by means of PCR and the primers SEQ ID No. 92 and SEQ ID No. 93 was eluted from the agarose gel using methods known per se and cleaved using the restriction enzymes NcoI and HindIII. From this, a 962 bp fragment resulted, which codes for a protein consisting of the entire primary sequence (SEQ ID No. 94). Using standard methods, the NcoI/HindIII-cleaved amplificate was cloned in the vector pCB97-30 and the clone pCB-gps was obtained.

Sequencing of the clone pCB-gps confirmed a sequence for the GGPP synthase from A. fulgidus which differs from the published sequence AF120272 in one nucleotide. By the insertion of an NcoI-cleavage site in the gps gene, the second codon of GGPP synthase was modified. In the published sequence AF120272, CTG (position 4-6) codes for leucine. By means of the amplification with the two primers SEQ ID No. 92 and SEQ ID No. 93, this second codon in GTG, which codes for valine, was modified.

The clone pCB-gps was therefore used for the cloning of the gps gene in the vector pMCL-CrtYIBZ/idi. The cloning was carried out by isolation of the KpnI/XhoI fragment from pCB-gps and ligation in the KpnI- and XhoI-cleaved vector pMCL-CrtYIBZ/idi. The cloned KpnI/XhoI fragment (SEQ ID No. 94) carries the Prrn16 promoter together with a minimal 5′-UTR sequence of rbcL, the first 6 codons of rbcL, which lengthen the GGPP synthase N-terminally, and 3′ from the gps gene the psbA sequence. The N terminus of the GGPP synthase thus has, instead of the natural amino acid sequence with Met-Leu-Lys-Glu (amino acid 1 to 4 from AF120272), the modified amino acid-sequence Met-Thr-Pro-Gin-Thr-Ala-Met-Val-Lys-Glu. It results from this that the recombinant GGPP synthase, beginning with Lys in position 3 (in AF120272) is identical and has no further modifications in the amino acid sequence. The rbcL and psbA sequences were used as in a reference according to Eibl et al. (Plant J. 19. (1999), 1-13). The resulting clone is called pMCL-CrtYIBZ/idi/gps.

### EXAMPLE 3

Biotransformation of Zeaxanthin in Recombinant E. coli Strains

For zeaxanthin biotransformation, recombinant E. coli strains are produced which are equipped for zeaxanthin production by heterologous complementation. Strains of E. coli TOP10 were used as host cells for the complementation experiments with the plasmids pNOSTF-G and pMCL-CrtYIBZ/idi/gps.

In order to produce E. coli strains which make possible the synthesis of zeaxanthin in high concentration, the plasmid pMCL-CrtYIBZ/idi/gps was constructed. The plasmid carries the biosynthesis genes crtY, crtB, crtI and crtY of Erwinia uredovora, the gene gps (for geranylgeranyl pyrophosphate synthastase) from Archaeoglobus fulgidus and the gene idi (isopentenyl phosphate isomerase) from E. coli. With this construct, limiting steps for a high accumulation of carotenoids and their biosynthetic precursors were eliminated. This has been described beforehand by Wang et al. in a similar manner using several plasmids (Wang, C.-W., Oh, M.-K. and Liao, J. C.; Engineered isoprenoid pathway enhances astaxanthin production in Escherichia coli, Biotechnology and Bioengineering 62 (1999), 235-241).

Cultures of E. coli TOP10 were transformed in a manner known per se with the two plasmids pNOSTF-G and pMCL-CrtYIBZ/idi/gps and cultured overnight in LB medium at 30° C. or 37° C. Ampicillin (50 μg/ml), chloramphenicol (50 μg/ml) and isopropyl β-thiogalactoside (1 mmol) were likewise added overnight in a manner customary per se.

For the isolation of the carotenoids from the recombinant strains, the cells were extracted with acetone, the organic solvent was evaporated to dryness and the carotenoids were separated by means of HPLC on a C30 column. The following process conditions were set.

Separating column: Prontosil C30 column, 250×4.6 mm, (Bischoff, Leonberg)

Flow rate: 1.0 ml/min

Eluents: Eluent A—100% methanol

Eluent B—80% methanol, 0.2% ammonium acetate
Eluent C—100% t-butyl methyl ether

Gradient Profile:

| Time | Flow rate | % eluent A | % eluent B | % eluent C |
|---|---|---|---|---|
| 1.00 | 1.0 | 95.0 | 5.0 | 0 |
| 1.05 | 1.0 | 80.0 | 5.0 | 15.0 |
| 14.00 | 1.0 | 42.0 | 5.0 | 53.0 |
| 14.05 | 1.0 | 95.0 | 5.0 | 0 |
| 17.00 | 1.0 | 95.0 | 5.0 | 0 |
| 18.00 | 1.0 | 95.0 | 5.0 | 0 |

Detection: 300-500 nm

The spectra were determined directly from the elution peaks using a photodiode array detector. The substances isolated were identified by means of their absorption spectra and their retention times in comparison with standard samples.

### EXAMPLE 4

Analogously to the previous examples, an E. coli strain was prepared which expresses a ketolase from Haematococcus pluvialis Flotow em. Wille. To this end, the cDNA which codes for the entire primary sequence of the ketolase from Haematococcus pluvialis Flotow em. Wille was amplified and cloned in the same expression vector as in Example 1.

The cDNA which codes for the ketolase from Haematococcus pluvialis was amplified by means of PCR of a Haematococcus pluvialis (strain 192.80 of the “Collection of algal cultures of the University of Göttingen”) suspension culture. For the preparation of total RNA from a suspension culture of Haematococcus pluvialis (strain 192.80), which had been grown for 2 weeks with indirect daylight at room temperature in Haematococcus medium (1.2 g/l of sodium acetate, 2 g/l of yeast extract, 0.2 g/l of MgCl2×6H₂O, 0.02 CaCl2×2H₂O; pH 6.8; after autoclaving addition of 400 mg/l of L-asparagine, 10 mg/l of FeSO4×H₂O), the cells were harvested, frozen in liquid nitrogen and pulverized in the mortar. Subsequently, 100 mg of the frozen, pulverized algal cells were transferred to a reaction vessel and taken up in 0.8 ml of Trizol buffer (LifeTechnologies). The suspension was extracted with 0.2 ml of chloroform. After centrifugation at 12 000 g for 15 minutes, the aqueous supernatant was removed and transferred to a new reaction vessel and extracted with one volume of ethanol. The RNA was precipitated with one volume of isopropanol, washed with 75% ethanol and the pellet was dissolved in DEPC water (overnight incubation of water with 1/1000 volume of diethyl pyrocarbonate at room temperature, subsequently autoclaved). The RNA concentration was determined photometrically.

For the cDNA synthesis, 2.5 ug of total RNA were denatured for 10 min at 60° C., cooled for 2 min on ice and transcribed in cDNA by means of a cDNA kit (Ready-to-go-you-prime-beads, Pharmacia Biotech) according to the manufacturer's instructions using an antisense-specific primer PR1 (gcaagctcga cagctacaaa cc).

The nucleic acid encoding a ketolase from Haematococcus pluvialis (strain 192.80) was amplified by means of polymerase chain reaction (PCR) of Haematococcus pluvialis using a sense-specific primer PR2 (gaagcatgca gctagcagcgacag) and an antisense-specific primer PR1.

The PCR conditions were as follows:

The PCR for the amplification of the cDNA which codes for a ketolase protein consisting of the total primary sequence was carried out in a 50 ml reaction batch, in which was comprised:
4 ml of a Haematococcus pluvialis cDNA (prepared as described above)
0.25 mM dNTPs
0.2 mM PR1
0.2 mM PR2
5 ml 10×PCR buffer (TAKARA)
0.25 ml R Taq polymerase (TAKARA)
25.8 ml dist. water

The PCR was carried out under the following cycle conditions:

1×94° C. 2 minutes

35×94° C. 1 minute

53° C. 2 minutes
72° C. 3 minutes
   1×72° C. 10 minutes

The PCR amplification with PR1 and PR2 resulted in a 1155 bp fragment which codes for a protein consisting of the entire primary sequence:

| | |
|---|---|
| gaagcatgca gctagcagcg acagtaatgt tggagcagct taccggaagc gctgaggcac | 60 |
| tcaaggagaa ggagaaggag gttgcaggca gctctgacgt gttgcgtaca tgggcgaccc | 120 |
| agtactcgct tccgtcagag gagtcagacg cggcccgccc gggactgaag aatgcctaca | 180 |
| agccaccacc ttccgacaca aagggcatca caatggcgct agctgtcatc ggctcctggg | 240 |
| ccgcagtgtt cctccacgcc atttttcaaa tcaagcttcc gacctccttg gaccagctgc | 300 |
| actggctgcc cgtgtcagat gccacagctc agctggttag cggcagcagc agcctgctgc | 360 |
| acatcgtcgt agtattcttt gtcctggagt tcctgtacac aggccttttt atcaccacgc | 420 |
| atgatgctat gcatggcacc atcgccatga gaaacaggca gcttaatgac ttcttgggca | 480 |
| gagtatgcat ctccttgtac gcctggtttg attacaacat gctgcaccgc aagcattggg | 540 |
| agcaccacaa ccacactggc gaggtgggca aggaccctga cttccacagg ggaaaccctg | 600 |
| gcattgtgcc ctggtttgcc agcttcatgt ccagctacat gtcgatgtgg cagtttgcgc | 660 |
| gcctcgcatg gtggacggtg gtcatgcagc tgctgggtgc gccaatggcg aacctgctgg | 720 |
| tgttcatggc ggccgcgccc atcctgtccg ccttccgctt gttctacttt ggcacgtaca | 780 |
| tgccccacaa gcctgagcct ggcgccgcgt caggctcttc accagccgtc atgaactggt | 840 |
| ggaagtcgcg cactagccag gcgtccgacc tggtcagctt tctgacctgc taccacttcg | 900 |
| acctgcactg ggagcaccac cgctggccct ttgccccctg gtgggagctg cccaactgcc | 960 |
| gccgcctgtc tggccgaggt ctggttcctg cctagctgga cacactgcag tgggccctgc | 1020 |
| tgccagctgg gcatgcaggt tgtggcagga ctgggtgagg tgaaaagctg caggcgctgc | 1080 |
| tgccggacac gctgcatggg ctaccctgtg tagctgccgc cactagggga gggggtttgt | 1140 |
| agctgtcgag cttgc | |

Using standard methods, the amplificate was cloned in the PCR cloning vector pGEM-Teasy (Promega) and the clone pGKETO2 was obtained.

Sequencing of the clone pGKETO2 with the T7 and the SP6 primer confirmed a sequence which differed only in the three codons 73,114 and 119 in one base each of the published sequence X86782. These nucleotide exchanges were reproduced in an independent amplification experiment and thus represent the nucleotide sequence in the Haematococcus pluvialis strain 192.80 used.

This clone was used for the expression of the ketolase of Haematococcus pluvialis. The transformation of the E. coli strains, their culturing and the analysis of the carotenoid profile was carried out as described in Example 3.

Table 1 shows a comparison of the amounts of carotenoid produced bacterially:

Table 1: Comparison of the bacterial ketocarotenoid synthesis when using two different ketolases, the NOST ketolase from Nostoc sp. PCC7120 (Example 1) and the ketolase from Haematococcus pluvialis (Example 4). Amounts of carotenoid are indicated in ng/ml of culture fluid.

| | Asta- | Adoni- | Adoni- | Cantha- | Zea- |
|---|---|---|---|---|---|
| Ketolase from | xanthin | rubin | xanthin | xanthin | xanthin |
| Haematococcus pluvialis | 13 | | 102 | | 738 |
| Flotow em. Wille | | | | | |
| Nostoc sp. Strain | 491 | 186 | | 120 | |
| PCC7120 | | | | | |

### EXAMPLE 5

Amplification of a DNA which Encodes the Total Primary Sequence of the NP196 Ketolase from Nostoc punctiforme ATCC 29133

The DNA which codes for the NP196 ketolase from Nostoc punctiforme ATCC 29133 was amplified by means of PCR from Nostoc punctiforme ATCC 29133 (strain of the “American Type Culture Collection”).

For the preparation of genomic DNA from a suspension culture of Nostoc punctiforme ATCC 29133 which had been grown for 1 week with continuous illumination and constant shaking (150 rpm) at 25° C. in BG 11 medium (1.5 g/l of NaNO₃, 0.04 g/l of K₂PO₄×3H₂O, 0.075 g/l of MgSO₄×H₂O, 0.036 g/l of CaCl₂×2H₂O, 0.006 g/l of citric acid, 0.006 g/l of ferric ammonium citrate, 0.001 g/l of EDTA disodium magnesium, 0.04 g/l of Na₂CO₃, 1 ml of Trace Metal Mix “A5+Co” (2.86 g/l of H₃BO₃, 1.81 g/l of MnCl₂×4H₂O, 0.222 g/l of ZnSO₄×7H₂O, 0.39 g/l of NaMoO₄×2H₂O, 0.079 g/l of CuSO₄×5H₂O, 0.0494 g/l of Co(NO₃)₂×6H₂O)), the cells were harvested by centrifugation, frozen in liquid nitrogen and pulverized in the mortar.

Protocol for the Isolation of DNA from Nostoc punctiforme ATCC 29133:

The bacterial cells from a 10 ml liquid culture were pelleted by centrifugation at 8000 rpm for 10 minutes. Subsequently, the bacterial cells were pulverized in liquid nitrogen with a mortar and ground. The cell material was resuspended in 1 ml 10 mM Tris HCl (pH 7.5) and transferred to an Eppendorf reaction vessel (2 ml volume). After addition of 100 ∝l of proteinase K (concentration: 20 mg/ml), the cell suspension was incubated for 3 hours at 37° C. Subsequently, the suspension was extracted with 500 μl of phenol. After centrifugation at 13 000 rpm for 5 minutes, the upper, aqueous phase was transferred to a new 2 ml Eppendorf reaction vessel. The extraction with phenol was repeated 3 times. The DNA was precipitated by addition of a 1/10 volume of 3 M sodium acetate (pH 5.2) and 0.6 volume of isopropanol and subsequently washed with 70% ethanol. The DNA pellet was dried at room temperature, taken up in 25 μl of l water and dissolved with heating at 65° C.

The nucleic acid encoding a ketolase from Nostoc punctiforme ATCC 29133 was amplified by means of “polymerase chain reaction” (PCR) of Nostoc punctiforme ATCC 29133 using a sense-specific primer (NP196-1, SEQ ID No. 100) and an antisense-specific primer (NP196-2 SEQ ID No. 101).

The PCR conditions were as follows:

The PCR for the amplification of the DNA which codes for a ketolase protein consisting of the total primary sequence was carried out in a 50 ul reaction batch, in which was comprised:
1 ul of a Nostoc punctiforme ATCC 29133 DNA (prepared as described above)
0.25 mM dNTPs
0.2 mM NP196-1 (SEQ ID No. 100)
0.2 mM NP196-2 (SEQ ID No. 101)
5 ul of 10×PCR buffer (TAKARA)
0.25 ul of R Taq polymerase (TAKARA)
25.8 ul of dist. water

The PCR was carried out under the following cycle conditions:

1×94° C. 2 minutes

35×94° C. 1 minute

55° C. 1 minutes
72° C. 3 minutes
   1×72° C. 10 minutes

The PCR amplification with SEQ ID No. 100 and SEQ ID No. 101 resulted in a 792 bp fragment which coded for a protein consisting of the entire primary sequence (NP196, SEQ ID No. 102). Using standard methods, the amplificate was cloned in the PCR-cloning vector pCR 2.1 (Invitrogen) and the clone pNP196 was obtained.

Sequencing of the clone pNP196 using the M13F and the M13R primer confirmed a sequence which is identical to the DNA sequence of 140,571-139,810 of the database entry NZ_AABC01000196 (inversely oriented to the published database entry) with the exception that G in position 140,571 was replaced by A in order to produce a standard start codon ATG. This nucleotide sequence was reproduced in an independent amplification experiment and thus represents the nucleotide sequence in the Nostoc punctiforme ATCC 29133 used.

This clone pNP196 was therefore used for cloning in the expression vector pJIT117 (Guerineau et al. 1988, Nucl. Acids Res. 16: 11380).

pJIT117 was modified by replacing the 35S terminator by the OCS terminator (Octopine synthase) of the Ti plasmid pTi15955 of Agrobacterium tumefaciens (database entry X00493 from position 12,541-12,350, Gielen et al. (1984) EMBO J. 3 835-846).

The DNA fragment which comprises the OCS terminator region was prepared by means of PCR using the plasmid pHELLSGATE (database entry AJ311874, Wesley et al. (2001) Plant J. 27 581-590, isolated from E. coli according to standard methods) and the primer OCS-1 (SEQ ID No. 133) and OCS-2 (SEQ ID No. 134).

The PCR conditions were as follows:

The PCR for the amplification of the DNA which comprises the octopine synthase (OCS) terminator region (SEQ ID No. 106) was carried out in a 50 ul reaction batch, in which were comprised:
100 ng of pHELLSGATE plasmid DNA
0.25 mM dNTPs
0.2 mM OCS-1 (SEQ ID No. 104)
0.2 mM OCS-2 (SEQ ID No. 105)
5 ul of 10×PCR buffer (Stratagene)
0.25 ul of Pfu polymerase (Stratagene)
28.8 ul of dist. water

The PCR was carried out under the following cycle conditions:

1×94° C. 2 minutes

35×94° C. 1 minute

50° C. 1 minute
72° C. 1 minute
   1×72° C. 10 minutes

The 210 bp amplificate was cloned using standard methods in the PCR cloning vector pCR 2.1 (Invitrogen) and the plasmid pOCS was obtained.

Sequencing of the clone pOCS confirmed a sequence which corresponded to a sequence section on the Ti plasmid pTi15955 of Agrobacterium tumefaciens (database entry X00493) from position 12,541 to 12,350.

The cloning was carried out by isolation of the 210 bp SalI-XhoI fragment from pOCS and ligation in the SalI-XhoI-cleaved vector pJIT117.

This clone is called pJO and was therefore used for cloning in the expression vector pJONP196.

The cloning was carried out by isolation of the 782 Bp SphI fragment from pNP196 and ligation in the SphI-cleaved vector pJO. The clone which comprises the NP196 ketolase of Nostoc punctiforme in the correct orientation as the N-terminal translational fusion with the rbcS transit peptide is called pJONP196.

### EXAMPLE 6

Preparation of Expression Vectors for the Constitutive Expression of the NP196 Ketolase from Nostoc punctiforme ATCC 29133 in Lycopersicon esculentum and Tagetes erecta.

The expression of the NP196 ketolase from Nostoc punctiforme in L. esculentum and in Tagetes erecta was carried out under the control of the constitutive promoter FNR (ferredoxin-NADPH oxidoreductase, database entry AB011474 position 70127 to 69493; WO03/006660), from Arabidopsis thaliana. The FNR gene begins at base pair 69492 and is annotated by “ferredoxin-NADP+ reductase”. The expression was carried out with the transit peptide rbcS from pea (Anderson et al. 1986, Biochem J. 240:709-715).

The DNA fragment which comprises the FNR promoter region from Arabidopsis thaliana was prepared by means of PCR using genomic DNA (isolated according to standard methods from Arabidopsis thaliana) and also the primers FNR-1 (SEQ ID No. 107) and FNR-2 (SEQ ID No. 108).

The PCR conditions were as follows:

The PCR for the amplification of the DNA which comprises the FNR promoter fragment FNR (SEQ ID No. 109) was carried out in a 50 ul reaction batch, in in which was comprised:
100 ng of genomic DNA from A. thaliana
0.25 mM dNTPs
0.2 mM FNR-1 (SEQ ID No. 107)
0.2 mM FNR-2 (SEQ ID No. 108)
5 ul of 10×PCR buffer (Stratagene)
0.25 ul of Pfu polymerase (Stratagene)
28.8 ul of dist. water

The PCR was carried out under the following cycle conditions:

1×94° C. 2 minutes

35×94° C. 1 minute

50° C. 1 minute
72° C. 1 minute
   1×72° C. 10 minutes

The 652 bp amplificate was cloned using standard methods in the PCR cloning vector pCR 2.1 (Invitrogen) and the plasmid pFNR was obtained.

Sequencing of the clone pFNR confirmed a sequence which corresponded to a sequence section on chromosome 5 of Arabidopsis thaliana (database entry AB011474) from position 70127 to 69493.

This clone is called pFNR and was therefore used for cloning in the expression vector pJONP196 (described in Example 5).

The cloning was carried out by isolation of the 644 bp SmaI-HindIII fragment from pFNR and ligation in the Ecl13611-HindIII-cleaved vector pJONP196. The clone which comprises the promoter FNR instead of the original promoter d35S and the fragment NP196 in the correct orientation as the N-terminal fusion with the rbcS transit peptide is called pJOFNR:NP196.

The preparation of an expression cassette for the Agrobacterium-mediated transformation of the NP196 ketolase from Nostoc in L. esculentum was carried out using the binary vector pSUN3 (WO02/00900).

For the preparation of the expression vector MSP105, the 1,839 bp EcoRI-XhoI fragment from pJOFNR:NP196 was ligated with the EcoRI-XhoI-cleaved vector pSUN3. The expression vector MSP105 comprises fragment FNR promoter the FNR promoter (635 bp), fragment rbcS TP FRAGMENT the rbcS transit peptide from pea (194 bp), fragment NP196 KETO CDS (761 bp), coding for the Nostoc punctiforme NP196 ketolase, fragment OCS terminator (192 bp) the polyadenylation signal of the octopine synthase.

The preparation of an expression cassette for the Agrobacterium-mediated transformation of the expression vector with the NP196 ketolase from Nostoc punctiforme in Tagetes erecta was carried out using the binary vector pSUN5 (WO02/00900).

For the preparation of the Tagetes expression vector MSP106, the 1,839 bp EcoRI-XhoI fragment from pJOFNR:NP196 was ligated with the EcoRI-XhoI-cleaved vector pSUN5. MSP106 comprises fragment FNR promoter the FNR promoter (635 bp), fragment rbcS TP FRAGMENT the rbcS transit peptide from pea (194 bp), fragment NP196 KETO CDS (761 bp), coding for the Nostoc punctiforme NP196 ketolase, fragment OCS terminator (192 bp) the polyadenylation signal of octopine synthase.

### EXAMPLE 7

Preparation of Expression Vectors for the Flower-Specific Expression of the NP196 Ketolase from Nostoc punctiforme ATCC 29133 in Lycopersicon esculentum and Tagetes erecta

The expression of the NP196 ketolase from Nostoc punctiforme in L. esculentum and Tagetes erecta was carried out with the transit peptide rbcS from pea (Anderson et al. 1986, Biochem J. 240:709-715). The expression was carried out under the control of the flower-specific promoter EPSPS from Petunia hybrida (database entry M37029: nucleotide region 7-1787; Benfey et al. (1990) Plant Cell 2: 849-856).

The DNA fragment which comprises the EPSPS promoter region (SEQ ID No. 112) from Petunia hybrida was prepared by means of PCR using genomic DNA (isolated according to standard methods from Petunia hybrida) and the primer EPSPS-1 (SEQ ID No. 110) and EPSPS-2 (SEQ ID No. 111).

The PCR conditions were as follows:

The PCR for the amplification of the DNA which comprises the EPSPS promoter fragment (database entry M37029: nucleotide region 7-1787) was carried out in a 50 μl reaction batch, in which was comprised:
100 ng of genomic DNA from A. thaliana
0.25 mM dNTPs
0.2 mM EPSPS-1 (SEQ ID No. 110)
0.2 mM EPSPS-2 (SEQ ID No. 111)
5 ul of 10×PCR buffer (Stratagene)
0.25 ul of Pfu polymerase (Stratagene)
28.8 ul of dist. water

The PCR was carried out under the following cycle conditions:

1×94° C. 2 minutes

35×94° C. 1 minute

50° C. 1 minute
72° C. 2 minutes
   1×72° C. 10 minutes

The 1773 Bp amplificate was cloned using standard methods in the PCR cloning vector pCR 2.1 (Invitrogen) and the plasmid pEPSPS obtained.

Sequencing of the clone pEPSPS confirmed a sequence which only differed by two deletion (bases ctaagtttcagga in position 46-58 of the sequence M37029; bases aaaaatat in position 1422-1429 of the sequence M37029) and the base exchanges (T instead of G in position 1447 of the sequence M37029; A instead of C in position 1525 of the sequence M37029; A instead of G in position 1627 of the sequence M37029) from the published EPSPS sequence (database entry M37029: nucleotide region 7-1787). The two deletions and the two base exchanges in the positions 1447 and 1627 of the sequence M37029 were reproduced in an independent amplification experiment and thus represent the actual nucleotide sequence in the Petunia hybrida plants used.

The clone pEPSPS was therefore used for cloning in the expression vector pJONP196 (described in Example 5).

The cloning was carried out by isolation of the 1763 bp SacI-HindIII fragment from pEPSPS and ligation in the SacI-HindIII-cleaved vector pJONP196. The clone which comprises the promoter EPSPS instead of the original promoter d35S is called pJOESP:NP196. This expression cassette comprises the fragment NP196 in the correct orientation as the N-terminal fusion with the rbcS transit peptide.

The preparation of an expression vector for the Agrobacterium-mediated transformation of the EPSPS-controlled NP196 ketolase from Nostoc punctiforme ATCC 29133 in L. esculentum was carried out using the binary vector pSUN3 (WO02/00900).

For the preparation of the expression vector MSP107, the 2.961 KB bp SacI-XhoI fragment from pJOESP:NP196 was ligated with the SacI-XhoI-cleaved vector pSUN3. The expression vector MSP107 comprises fragment EPSPS the EPSPS promoter (1761 bp), fragment rbcS TP FRAGMENT the rbcS transit peptide from pea (194 bp), fragment NP196 KETO CDS (761 bp), coding for the Nostoc punctiforme NP196 ketolase, fragment OCS terminator (192 bp) the polyadenylation signal of octopine synthase.

The preparation of an expression vector for the Agrobacterium-mediated transformation of the EPSPS-controlled NP196 ketolase from Nostoc punctiforme in Tagetes erecta was carried out using the binary vector pSUN5 (WO02/00900).

For the preparation of the expression vector MSP108, the 2,961 KB bp SacI-XhoI fragment from pJOESP:NP196 was ligated with the SacI-XhoI cleaved vector pSUN5. The expression vector MSP108 comprises fragment EPSPS the EPSPS promoter (1761 bp), fragment rbcS TP FRAGMENT the rbcS transit peptide from pea (194 bp), fragment NP196 KETO CDS (761 bp), coding for the Nostoc punctiforme NP196 ketolase, fragment OCS terminator (192 bp) the polyadenylation signal of octopine synthase.

### EXAMPLE 8

Amplification of a DNA which Encodes the Total Primary Sequence of the NP195 Ketolase from Nostoc punctiforme ATCC 29133.

The DNA which codes for the NP195 ketolase from Nostoc punctiforme ATCC 29133 was amplified by means of PCR from Nostoc punctiforme ATCC 29133 (strain of the “American Type Culture Collection”). The preparation of genomic DNA from a suspension culture of Nostoc punctiforme ATCC 29133 was described in Example 5.

The nucleic acid encoding a ketolase from Nostoc punctiforme ATCC 29133 was amplified by means of “polymerase chain reaction” (PCR) from Nostoc punctiforme ATCC 29133 using a sense-specific primer (NP195-1, SEQ ID No. 113) and an antisense-specific primer (NP195-2 SEQ ID No. 114).

The PCR conditions were as follows:

The PCR for the amplification of the DNA which codes for a ketolase protein consisting of the total primary sequence was carried out in a 50 ul reaction batch, in which was comprised:
1 ul of a Nostoc punctiforme ATCC 29133 DNA (prepared as described above)
0.25 mM dNTPs
0.2 mM NP195-1 (SEQ ID No. 113)
0.2 mM NP195-2 (SEQ ID No. 114)
5 ul of 10×PCR buffer (TAKARA)
0.25 ul of R Taq polymerase (TAKARA)
25.8 ul of dist. water

The PCR was carried out under the following cycle conditions:

1×94° C. 2 minutes

35×94° C. 1 minute

55° C. 1 minutes
72° C. 3 minutes
   1×72° C. 10 minutes

The PCR amplification with SEQ ID No. 113 and SEQ ID No. 114 resulted in an 819 bp fragment which codes for a protein consisting of the total primary sequence (NP195, SEQ ID No. 115). Using standard methods, the amplificate was cloned in the PCR cloning vector pCR 2.1 (Invitrogen) and the clone pNP195 obtained.

Sequencing of the clone pNP195 with the M13F and the M13R primer confirmed a sequence which is identical with the DNA sequence from 55,604-56,392 of the database entry NZ_AABC010001965, with the exception that T in position 55,604 was replaced by A in order to produce a standard start codon ATG. This nucleotide sequence was reproduced in an independent amplification experiment and thus represents the nucleotide sequence in the Nostoc punctiforme ATCC 29133 used.

This clone pNP195 was therefore used for cloning in the expression vector pJO (described in Example 5). The cloning was carried out by isolation of the 809 Bp SphI fragment from pNP195 and ligation in the SphI-cleaved vector pJO. The clone which comprises the NP195 ketolase from Nostoc punctiforme in the correct orientation as the N-terminal translational fusion with the rbcS transit peptide is called pJONP195.

### EXAMPLE 9

Preparation of Expression Vectors for the Constitutive Expression of the NP195 Ketolase from Nostoc punctiforme ATCC 29133 in Lycopersicon esculentum and Tagetes erecta.

The expression of the NP195 ketolase from Nostoc punctiforme in L. esculentum and in Tagetes erecta was carried out under the control of the constitutive promoter FNR (ferredoxin-NADPH oxidoreductase, database entry AB011474 position 70127 to 69493; WO03/006660), from Arabidopsis thaliana. The FNR gene begins at base pair 69492 and is annotated by “ferredoxin-NADP+ reductase”. The expression was carried out with the transit peptide rbcS from pea (Anderson et al. 1986, Biochem J. 240:709-715).

The clone pFNR (described in Example 6) was therefore used for cloning in the expression vector pJONP195 (described in Example 8).

The cloning was carried out by isolation of the 644 bp Sma-HindIII fragment from pFNR and ligation in the Ecl13611-HindIII-cleaved vector pJONP195. The clone, which comprises the promoter FNR instead of the original promoter d35S and the fragment NP195 in the correct orientation as the N-terminal fusion with the rbcS transit peptide, is called pJOFNR:NP195.

The preparation of an expression cassette for the Agrobacterium-mediated transformation of the NP195 ketolase from Nostoc punctiforme in L. esculentum was carried out using the binary vector pSUN3 (WO02/00900).

For the preparation of the expression vector MSP109, the 1,866 bp EcoRI-XhoI fragment from pJOFNR:NP195 was ligated with the EcoRI-XhoI-cleaved vector pSUN3. The expression vector MSP109 comprises fragment FNR promoter the FNR promoter (635 bp), fragment rbcS TP FRAGMENT the rbcS transit peptide from pea (194 bp), fragment NP195 KETO CDS (789 bp), coding for the Nostoc punctiforme NP195 ketolase, fragment OCS terminator (192 bp) the polyadenylation signal from the octopine synthase.

The preparation of an expression cassette for the Agrobacterium-mediated transformation of the expression vector with the NP195 ketolase from Nostoc punctiforme punctiforme in Tagetes erecta was carried out using the binary vector pSUN5 (WO 02/00900).

For the preparation of the Tagetes expression vector MSP10, the 1,866 bp EcoRI-XhoI fragment from pJOFNR:NP195 was ligated with the EcoRI-XhoI-cleaved vector pSUN5. The expression vector MSP110 comprises fragment FNR promoter the FNR promoter (635 bp), fragment rbcS TP FRAGMENT the rbcS transit peptide from pea (194 bp), fragment NP195 KETO CDS (789 bp), coding for the Nostoc punctiforme NP195 ketolase, fragment OCS terminator (192 bp) the polyadenylation signal of octopine synthase.

### EXAMPLE 10

Preparation of Expression Vectors for the Flower-Specific Expression of the NP195 Ketolase from Nostoc punctiforme ATCC 29133 in Lycopersicon esculentum and Tagetes erecta.

The expression of the NP195 ketolase from Nostoc punctiforme in L. esculentum and Tagetes erecta was carried out with the transit peptide rbcS from pea (Anderson et al. 1986, Biochem J. 240:709-715). The expression was carried out under the control of the flower-specific promoter EPSPS from Petunia hybrida (database entry M37029: nucleotide region 7-1787; Benfey et al. (1990) Plant Cell 2: 849-856).

The clone pEPSPS (described in Example 7) was therefore used for cloning in the expression vector pJONP195 (described in Example 8).

The cloning was carried out by isolation of the 1763 Bp SacI-HindIII fragment from pEPSPS and ligation in the SacI-HindIII-cleaved vector pJONP195. The clone, which comprises the promoter EPSPS instead of the original promoter d35S, is called pJOESP:NP195. This expression cassette comprises the fragment NP195 in the correct orientation as the N-terminal fusion with the rbcS transit peptide.

The preparation of an expression vector for the Agrobacterium-mediated transformation of the EPSPS-controlled NP195 ketolase from Nostoc punctiforme ATCC 29133 in L. esculentum was carried out using the binary vector pSUN3 (WO02/00900).

For the preparation of the expression vector MSP111, the 2,988 KB bp SacI-XhoI fragment from pJOESP:NP195 was ligated with the SacI-XhoI-cleaved vector pSUN3. The expression vector MSP111 comprises fragment EPSPS the EPSPS promoter (1761 bp), fragment rbcS TP FRAGMENT the rbcS transit peptide from pea (194 bp), fragment NP195 KETO CDS (789 bp), coding for the Nostoc punctiforme NP195 ketolase, fragment OCS terminator (192 bp) the polyadenylation signal of octopine synthase.

The preparation of an expression vector for the Agrobacterium-mediated transformation of the EPSPS-controlled NP195 ketolase from Nostoc punctiforme in Tagetes erecta was carried out using the binary vector pSUN5 (WO02/00900).

For the preparation of the expression vector MSP112, the 2,988 KB bp SacI-XhoI fragment from pJOESP:NP195 was ligated with the SacI-XhoI-cleaved vector pSUN5. The expression vector MSP112 comprises fragment EPSPS the EPSPS promoter (1761 bp), fragment rbcS TP FRAGMENT the rbcS transit peptide from pea (194 bp), fragment NP195 KETO CDS (789 bp), coding for the Nostoc punctiforme NP195 ketolase, fragment OCS terminator (192 bp) the polyadenylation signal of octopine synthase.

### EXAMPLE 11

Preparation of an Expression Cassette for the Flower-Specific Overexpression of the Chromatoplast-Specific Beta-Hydroxylase from Lycopersicon esculentum.

The expression of the chromatoplast-specific beta-hydroxylase from Lycopersicon esculentum in Tagetes erecta is carried out under the control of the flower-specific promoter EPSPS from Petunia (Example 7). As the terminator element, LB3 from Vicia faba is used. The sequence of the chromatoplast-specific beta-hydroxylase was prepared by RNA isolation, reverse transcription and PCR.

For the preparation of the LB3 terminator sequence from Vicia faba, genomic DNA from Vicia faba tissue is isolated according to standard methods and employed by genomic PCR using the primers PR206 and PR207. The PCR for the amplification of this LB3 DNA fragment is carried out in a 50 ul reaction batch, in which is comprised:
1 ul of cDNA (prepared as described above)
0.25 mM dNTPs
0.2 uM PR206 (SEQ ID No. 116)
0.2 uM PR207 (SEQ ID No. 117)
5 ul of 10×PCR buffer (TAKARA)
0.25 ul of R Taq polymerase (TAKARA)
28.8 ul of dist. water

The PCR amplification with PR206 and PR207 results in a 0.3 kb fragment which comprises for the LB terminator. The amplificate is cloned in the cloning vector pCR-BluntII (Invitrogen). Sequencings with the primers T7 and M13 confirm a sequence identical to the sequence SEQ ID: 118. This clone is called pTA-LB3 and is therefore used for cloning in the vector pJIT117 (see below).

For the preparation of the beta-hydroxylase sequence, total RNA from tomato is prepared. To this end, 100 mg of the frozen, pulverized flowers are transferred to a reaction vessel and taken up in 0.8 ml of Trizol buffer (LifeTechnologies). The suspension is extracted with 0.2 ml of chloroform. After centrifugation at 12 000 g for 15 minutes, the aqueous supernatant is removed and transferred to a new reaction vessel and extracted with one volume of ethanol. The RNA is precipitated using one volume of isopropanol, washed with 75% ethanol and the pellet is dissolved in DEPC water (overnight incubation of water with a 1/1000 volume of diethyl pyrocarbonate at room temperature, subsequently autoclaved). The RNA concentration is determined photometrically. For the cDNA synthesis, 2.5 ug of total RNA are denatured for 10 min at 60° C., cooled for 2 min on ice and transcribed by means of a cDNA kit (Ready-to-go-you-prime-beads, Pharmacia Biotech) according to manufacturer's details using an antisense-specific primer (PR215 SEQ ID No. 119) in cDNA.

The conditions of the subsequent PCR reactions are as follows:

The PCR for the amplification of the VPR203-PR215 DNA fragment which codes for the beta-hydroxylase is carried out in a 50 ul reaction batch, in which was comprised:
1 ul of cDNA (prepared as described above)
0.25 mM dNTPs
0.2 uM VPR203 (SEQ ID No. 120)
0.2 uM PR215 (SEQ ID No. 119)
5 ul of 10×PCR buffer (TAKARA)
0.25 ul of R Taq polymerase (TAKARA)
28.8 ul of dist. water

The PCR amplification with VPR203 and PR215 results in a 0.9 kb fragment which codes for the beta-hydroxylase. The amplificate is cloned in the cloning vector pCR-BluntII (Invitrogen). Sequencings with the primers T7 and M13 confirm a sequence identical to the sequence SEQ ID No. 121. This clone is called pTA-CrtR-b2 and is therefore used for cloning in the vector pCSP02 (see below).

The EPSPS promoter sequence from Petunia is prepared by PCR amplification using the plasmid MSP107 (see Example 7) and the primers VPR001 and VPR002. The PCR for the amplification of this EPSPS-DNA fragment is carried out in a 50 ul reaction batch, in which is comprised:
1 ul of cDNA (prepared as described above)
0.25 mM dNTPs
0.2 uM VPR001 (SEQ ID No. 122)
0.2 uM VPR002 (SEQ ID No. 123)
5 ul of 10×PCR buffer (TAKARA)
0.25 ul of R Taq polymerase (TAKARA)
28.8 ul of dist. water

The PCR amplification with VPR001 and VPR002 results in a 1.8 kb fragment which encodes the EPSPS promoter. The amplificate is cloned in the cloning vector pCR-BluntII (Invitrogen). Sequencings with the primers T7 and M13 confirm a sequence identical to the sequence SEQ ID: 124. This clone is called pTA-EPSPS and is therefore used for cloning in the vector pCSP03 (see below).

The first cloning step is carried out by isolation of the 0.3 kb PR206-PR207 EcoRI-XhoI fragment from pTA-LB3, derived from the cloning vector pCR-BluntII (Invitrogen), and ligation with the EcoRI-XhoI-cleaved vector pJIT117. The clone, which comprises the 0.3 kb terminator LB3, is called pCSP02.

The second cloning step is carried out by isolation of the 0.9 kb VPR003-PR215 EcoRI-HindIII fragment from pTA-CrtR-b2, derived from the cloning vector pCR-BluntII (Invitrogen), and ligation with the EcoRI-HindIII-cleaved vector pCSP02. The clone, which comprises the 0.9 kb beta-hydroxylase fragment CrtR-b2, is called pCSP03. By means of the ligation, a transcriptional fusion results between the terminator LB3 and the beta-hydroxylase fragment CrtR-b2.

The third cloning step is carried out by isolation of the 1.8 kb VPR001-VPR002 NcoI-SacI fragment from pTA-EPSPS, derived from the cloning vector pCR-BluntII (Invitrogen), and ligation with the NcoI-SacI-cleaved vector pCSP03. The clone, which comprises the 1.8 kb EPSPS promoter fragment, is called pCSP04. By means of the ligation, a transcriptional fusion results between the EPSPS promoter and the beta-hydroxylase fragment CrtR-b2. pCSP04 comprises fragment fragment EPSPS (1792 bp) the EPSPS promoter, the fragment crtRb2 (929 bp) the beta-hydroxylase CrtRb2, fragment LB3 (301 bp) the LB3 terminator.

For the cloning of this hydroxylase-overexpression cassette in expression vectors for the Agrobacterium-mediated transformation of Tagetes erecta, the beta-hydroxylase cassette is isolated as the 3103 bp Ecl136II-XhoI fragment. The filling of the 3′ ends (30 min at 30° C.) is carried out according to standard methods (Klenow fill-in).

The expression vector is called pCSEbhyd

### EXAMPLE 12

Preparation of Expression Vectors for the Flower-Specific Expression of the Chromoplast-Specific Lycopene Beta-Ccyclase from Lycopersicon esculentum Under the Control of the Promoter P76 and for the Flower-Specific Expression of the Ketolase NP196 from Nostoc punctiforme ATCC 29133 Under the Control of the EPSPS Promoter

Isolation of promoter P76 (SEQ ID NO. 125) by means of PCR with genomic DNA from Arabidopsis thaliana as the matrix.

For this, the oligonucleotide primers P76for (SEQ ID NO. 126) and P76rev (SEQ ID NO. 127) were used. The oligonucleotides were provided during the synthesis with a 5′ phosphate residue.

P76 for5′-CCCGGGTGCCAAAGTAACTCTTTAT-3′
P76 rev 5′-GTCGACAGGTGCATGACCAAGTAAC-3′

The genomic DNA was isolated from Arabidopsis thaliana as described (Galbiati M et al. Funct. Integr. Genomics 2000, 20 1:25-34).

The PCR amplification was carried out as follows:

80 ng of genomic DNA

1× Expand Long Template PCR buffer

2.5 mM MgCl2

350 μM each dATP, dCTP, dGTP, dTTp

300 nM each of each primer

2.5 units of Expand Long Template Polymerase

in a final volume of 25 μl

The following temperature program is used:

1 cycle with 120 sec at 94° C.

35 cycles with 94° C. for 10 sec,

48° C. for 30 sec and

68° C. for 3 min

1 cycle with 68° C. for 10 min

The PCR product is separated using agarose gel electrophoresis and the 1032 bp fragment is isolated by gel elution.

The vector pSun5 is digested with the restriction endonuclease EcoRV and likewise purified by means of agarose gel electrophoresis and recovered by gel elution.

The purified PCR product is cloned in the vector treated in this way.

This construct is designated by p76. The fragment 1032 bp long, which represents the promoter P76 from Arabidopsis, was sequenced (Seq ID NO. 131).

The terminator 35ST is obtained from pJIT 117 by digestion with the restriction endonucleases KpnI and SmaI. The 969 bp fragment resulting in this process is purified using agarose gel electrophoresis and isolated by gel elution.

The vector p76 is likewise digested with the restriction endonucleases KpnI and SmaI. The resulting 7276 bp fragment is purified using agarose gel electrophoresis and isolated by gel elution.

The 35ST fragment thus obtained is cloned in the p76 treated in this way.

The resulting vector is designated by p76_{—}35ST.

The isolation of the Bgene (SEQ ID NO. 128) was carried out by means of PCR with genomic DNA from Lycopersicon esculentum as the matrix.

For this, the oligonucleotide primers BgeneFor (SEQ ID NO. 129) and BgeneRev (SEQ ID NO. 130) were used. The oligonucleotides were provided in the synthesis with a 5′ phosphate residue.

SEQ ID NO 129

Bgenefor: 5′-CTATTGCTAGATTGCCAATCAG-3′:

SEQ ID NO 130

Bgenerev:5′-ATGGAAGCTCTTCTCAAG-3′

The genomic DNA was isolated from Lycopersicon esculentum as described (Galbiati M et al. Funct. Integr. Genomics 2000, 20 1:25-34).

The PCR amplification was carried out as follows:

80 ng of genomic DNA

1× Expand Long Template PCR buffer

2.5 mM MgCl2

350 μM each of dATP, dCTP, dGTP, dTTp

300 nM each of each primer

2.5 units of Expand Long Template Polymerase

in a final volume of 25 μl

The following temperature program was used:

1 cycle with 120 sec at 94° C.

35 cycles with 94° C. for 10 sec,

48° C. for 30 sec and

68° C. for 3 min

1 cycle with 68° C. for 10 min

The PCR product was purified using agarose gel electrophoresis and the 1665 bp fragment was isolated by gel elution.

The vector p76_{—}35ST is digested with the restriction endonuclease SmaI and likewise purified by means of agarose gel electrophoresis and recovered by gel elution.

The purified PCR product is cloned in the vector treated in this way.

This construct is designated by pB. The fragment 1486 bp long, which represents the Bgene from tomato, was sequenced and is identical in its nucleotide sequence with the database entry AF254793 (Seq ID NO. 1).

pB is digested with the restriction endonucleases PmeI and SspI and the 3906 bp fragment comprising the promoter P76, Bgene and the 35ST is purified by agarose gel electrophoresis and recovered by gel elution.

MSP108 (Example 7) is digested with the restriction endonuclease Ecl126II, purified by agarose gel electrophoresis and recovered by gel elution.

The purified 3906 bp fragment comprising the promoter P76, Bgene and the 35ST from pB is cloned in the vector MSP108 treated in this way.

This construct is designated by pMKP1.

### EXAMPLE 13

Preparation and Analysis of Transgenic Lycopersicon esculentum Plants

Transformation and regeneration of tomato plants was carried out according to the published method of Ling et al. (Plant Cell Reports (1998), 17:843-847). For the variety Microtom, selection was carried out using a higher kanamycin concentration (100 mg/l). As the starting explant for the transformation, cotyledons and hypocotyls of seedlings of the line Microtom seven to ten days old were used. For germination, the culture medium according to Murashige and Skoog (1962: Murashige and Skoog, 1962, Physiol. Plant 15, 473-) comprising 2% sucrose, pH 6.1 was used. Germination took place at 21° C. with little light (20 to 100 μE). After seven to ten days, the cotyledons were divided diagonally and the hypocotyls were cut into sections about 5 to 10 mm long and placed on the medium MSBN (MS, pH 6.1, 3% sucrose+1 mg/l of BAP, 0.1 mg/l of NAA), which was coated on the day before with suspension-cultured tomato cells. The tomato cells were covered with sterile filter paper in an air bubble-free manner. The preculture of the explants on the described medium was carried out for three to five days. Cells of the strain Agrobacterium tumefaciens LBA4404 were individually transformed with the plasmids. In each case, an overnight culture in YEB medium comprising kanamycin (20 mg/l) of the Agrobacterium strains individually transformed with the binary vectors was cultured at 28 degrees Celsius and the cells were centrifuged. The bacterial pellet was resuspended using liquid MS medium (3% sucrose, pH 6.1) and adjusted to an optical density of 0.3 (at 600 nm). The precultured explants were transferred to the suspension and incubated for 30 minutes at room temperature with slight shaking. Subsequently, the explants were dried using sterile filter paper and replaced on their preculture medium for the three-day coculture (21° C.).

After the coculture, the explants were transferred to MSZ2 medium (MS pH 6.1+3% sucrose, 2 mg/l of zeatin, 100 mg/l of kanamycin, 160 mg/l of timentin) and stored for selective regeneration at 21° C. under weak conditions (20 to 100 μE, light rhythm 16 h/8 h). Every two to three weeks, the transfer of the explants was carried out until sprouts are formed. It was possible to separate off small sprouts from the explant and to root them on MS (pH 6.1+3% sucrose) 160 mg/l of timentin, 30 mg/l of kanamycin, 0.1 mg/l of IAA. Rooted plants were transferred to the greenhouse.

According to the transformation method described above, with the following expression constructs the following lines were obtained:

With MSP105: msp105-1, msp105-2, msp105-3 was obtained

With MSP107: msp107-1, msp107-2, msp107-3 was obtained

With MSP109: msp109-1, msp109-2, msp109-3 was obtained

With MSP111: msp111-1, msp111′-2, msp111-3 was obtained

### EXAMPLE 14

Preparation of Transgenic Tagetes Plants

Tagetes seeds are sterilized and placed on germination medium (MS medium; Murashige and Skoog, Physiol. Plant. 15 (1962), 473-497) pH 5.8, 2% sucrose). Germination is carried out in a temperature/light/time interval of 18 to 28° C./20-200 μE/3 to 16 weeks, but preferably at 21° C., 20 to 70 mE, for 4 to 8 weeks.

All leaves of the in vitro plants developed by then are harvested and cut transversely to the center rib. The leaf explants resulting thereby having a size of 10 to 60 mm² are stored in the course of the preparation in liquid MS medium at room temperature for at most 2 hours.

Any desired Agrobacterium tumefaciens strain, but preferably a supervirulent strain, such as, for example, EHA 05 with an appropriate binary plasmid, which can comprise a selection marker gene (preferably bar or pat) and one or more trait or reporter genes, is cultured overnight and used for the coculturing with the leaf material. The culturing of the bacterial strain can be carried out as follows: An individual colony of the appropriate strain is inoculated into YEB (0.1% yeast extract, 0.5% beef extract, 0.5% peptone, 0.5% sucrose, 0.5% magnesium sulfate×7H₂O) with 25 mg/l of kanamycin and cultured at 28° C. for 16 to 20 hours. Subsequently, the bacterial suspension is harvested by centrifugation at 6000 g for 10 min and resuspended in liquid MS medium in such a way that an OD₆₀₀ of about 0.1 to 0.8 resulted. This suspension is used for the coculturing with the leaf material.

Immediately before the coculturing, the MS medium in which the leaves have been stored is replaced by the bacterial suspension. The incubation of the leaves in the Agrobacteria suspension was carried out for 30 min with slight shaking at room temperature. Subsequently, the infected explants are placed on an agar (e.g. 0.8% plant agar (Duchefa, NL)-solidified MS medium comprising growth regulators, such as, for example, 3 mg/l of benzylaminopurine (BAP) and 1 mg/l of indolylacetic acid (IAA). The orientation of the leaves on the medium is insignificant. The culturing of the explants takes place for 1 to 8 days, but preferably for 6 days, in this connection the following conditions can be used: light intensity: 30 to 80 μMol/m²×sec, temperature: 22 to 24° C., light/dark change of 16/8 hours. Subsequently, the cocultured explants are transferred to fresh MS medium, preferably comprising the same growth regulators, this second medium additionally comprising an antibiotic for suppression of the bacterial growth. Timentin in a concentration of 200 to 500 mg/l is very suitable for this purpose. As a second selective component, one for the selection of the transformation results is employed. Phosphinothricin in a concentration of 1 to 5 mg/l selects very efficiently, but other selective components are also conceivable according to the process to be used.

After one to three weeks in each case, the transfer of the explants to fresh medium is carried out until sprout buds and small sprouts develop, which are then transferred for rooting to the same basal medium including timentin and PPT or alternative components with growth regulators, namely, for example, 0.5 mg/l of indolylbutyric acid (IBA) and 0.5 mg/l of gibberelic acid GA₃. Rooted sprouts can be transferred to the greenhouse.

In addition to the methods described, the following advantageous modifications are possible:
Before the explants are infected with the bacteria, they can be preincubated for 1 to 12 days, preferably 3 to 4, on the medium described above for the coculture. Subsequently, the infection, coculture and selective regeneration is carried out as described above.
The pH for the regeneration (normally 5.8) can be lowered to pH 5.2. The control of the growth of the Agrobacteria is thereby improved.
The addition of AgNO₃ (3 to 10 mg/l) to the regeneration medium improves the condition of the culture including the regeneration itself.
Components which reduce the phenol formation and are known to the person skilled in the art, such as, for example, citric acid, ascorbic acid, PVP and very many others, have a positive effect on the culture.
Liquid culture medium can also be used for the total process. The culture can also be incubated on commercially available carriers, which are are positioned on the liquid medium.

According to the transformation method described above, the following lines were obtained with the following expression constructs:

With MSP106 was obtained: msp106-1, msp106-2, msp106-3

With MSP108 was obtained: msp108-1, msp108-2, msp108-3

With MSP110 was obtained: msp110-1, msp110-2, msp110-3

With MSP112 was obtained: msp112-1, msp112-2, msp112-3

With pCSEbhyd was obtained: csebhyd-1, csebhyd-2, csebhyd-3.

With pMKP1. was obtained: mkp1-1, mkp1-2, mkp1-3.

### EXAMPLE 15

### Enzymatic Lipase-Catalyzed Hydrolysis of Carotenoid Esters from Plant Material and Identification of the Carotenoids

General Working Procedure

a) Plant material ground in a mortar (e.g. petal material) (30-100 mg fresh weight) is extracted with 100% acetone (three times 500 μl; in each case shake for approximately 15 minutes). The solvent is evaporated. Carotenoids are subsequently taken up in 495 μl of acetone, 4.95 ml of potassium phosphate buffer (100 mM, pH 7.4) are added and the solutions are well mixed. The addition of about 17 mg of bile salts (Sigma) and 149 μl of an NaCl/CaCl₂ solution (3M NaCl and 75 mM CaCl₂) is then carried out. The suspension is incubated for 30 minutes at 37° C. For the enzymatic hydrolysis of the carotenoid esters, 595 μl of a lipase solution (50 mg/ml of lipase Type 7 from Candida rugosa (Sigma)) are added and incubated with shaking at 37C. After approximately 21 hours, an addition of 595 μl of lipase with fresh incubation of at least 5 hours at 37° C. was carried out again. Subsequently, approximately about 700 mg of Na₂SO₄ are dissolved in the solution. After addition of 1800 μl of petroleum ether, the carotenoids are extracted into the organic phase by vigorous mixing. This extraction by shaking is repeated until the organic phase remains colorless. The petroleum ether fractions are combined and the petroleum ether is evaporated. Free carotenoids are taken up in 100-120 μl of acetone. By means of HPLC and a C30 reverse-phase column, free carotenoids can be identified on the basis of retention time and UV-VIS spectra.

The bile salts or bile acid salts used are 1:1 mixtures of cholate and deoxycholate.

b) Working Procedure for Working Up if Only Small Amounts of Carotenoid Esters are Present in the Plant Material

Alternatively, the hydrolysis of the carotenoid esters can be achieved by lipase from Candida rugosa after separation by means of thin layer chromatography. To this end, 50-100 mg of plant material are extracted three times with approximately 750 μl of acetone. The solvent extract is concentrated in vacuo in a rotrary evaporator (increased temperatures of 40-50° C. are tolerable). Addition of 300 μl of petroleum ether:acetone (ratio 5:1) and thorough mixing is then carried out. Suspended substances are sedimented by centrifugation (1-2 minutes). The upper phase is transferred to a new reaction vessel. The residue remaining is again extracted with 200 μl of petroleum ether: acetone (ratio 5:1) and suspended substances are removed by centrifugation. The two extracts are brought together (volume 500 μl) and the solvents are evaporated. The residue is resuspended in 30 μl of petroleum ether:acetone (ratio 5:1) and applied to a thin layer plate (silica gel 60, Merck). If more than one application is necessary for preparative/analytical purposes, several aliquots in each case having a fresh weight of 50-100 mg should be prepared in the manner described for the thin layer chromatographic separation.

The thin layer plate is developed in petroleum ether:acetone (ratio 5:1). Carotenoid bands can be identified visually on account of their color. Individual carotenoid bands are scraped off and can be pooled for preparative/analytical purposes. Using acetone, the carotenoids are eluted from the silica material; the solvent is evaporated in vacuo. For the hydrolysis of the carotenoid esters, the residue is dissolved in 495 μl of acetone, 17 mg of bile salts (Sigma), 4.95 ml of 0.1M potassium phosphate buffer (pH 7.4) and 149 μl (3M NaCl, 75 mM CaCl₂) are added. After thorough mixing, the solution is equilibrated for 30 min at 37° C. The addition of 595 μl of lipase of Candida rugosa (Sigma, stock solution of 50 mg/ml in 5 mM CaCl₂) is then carried out. Overnight, the incubation with lipase with shaking at 37° C. is carried out. After approximately 21 hours, the same amount of lipase is added again; the mixture is incubated again at 37° C. with shaking for at least 5 hours. The addition of 700 mg of Na₂SO₄ (anhydrous) is then carried out; the mixture is extracted by shaking with 1800 μl of petroleum ether for about 1 minute and the mixture is centrifuged at 3500 revolutions/minute for 5 minutes. The upper phase is transferred to a new reaction vessel and the extraction with shaking is repeated until the upper phase is colorless. The combined petroleum ether phase is concentrated in vacuo (temperatures of 40-50° C. are possible). The residue is dissolved in 120 μl of acetone, if necessary by means of ultrasound. The dissolved carotenoids can be separated by means of HPLC using a C30 column and quantified with the aid of reference substances.

### EXAMPLE 16

### HPLC Analysis of Free Carotenoids

The analysis of the samples obtained according to the working procedures in Example 15 is carried out under the following conditions:

The following HPLC conditions were set.

Separating column: Prontosil C30 column, 250×4.6 mm, (Bischoff, Leonberg, Germany)

Flow rate: 1.0 ml/min

Eluents: Eluent A—100% methanol

Eluent B—80% methanol, 0.2% ammonium acetate
Eluent C—100% t-butyl methyl ether
   Detection: 300-530 nm

Gradient Profile:

| Time | Flow rate | % eluent A | % eluent B | % eluent C |
|---|---|---|---|---|
| 1.00 | 1.0 | 95.0 | 5.0 | 0 |
| 12.00 | 1.0 | 95.0 | 5.0 | 0 |
| 12.10 | 1.0 | 80.0 | 5.0 | 15.0 |
| 22.00 | 1.0 | 76.0 | 5.0 | 19.0 |
| 22.10 | 1.0 | 66.5 | 5.0 | 28.5 |
| 38.00 | 1.0 | 15.0 | 5.0 | 80.0 |
| 45.00 | 1.0 | 95.0 | 5.0 | 0 |
| 46.0 | 1.0 | 95.0 | 5.0 | 0 |

Some typical retention times for carotenoids formed according to the invention are, for example:

violaxanthin 11.7 min, astaxanthin 17.7 min, adonixanthin 19 min, adonirubin 19.9 min, zeaxanthin 21 min.

1. A process for the preparation of a ketocarotenoid by culturing a genetically modified, nonhuman organism, which in comparison with the wild-type has a modified ketolase activity and a modified β-cyclase activity, and the modified β-cyclase activity is caused by a β-cyclase comprising the amino acid sequence SEQ ID NO: 2 or a sequence derived from this sequence by substitution, insertion or deletion of amino acids, which has an identity of at least 70% at the amino acid level with the sequence SEQ ID NO: 2.

2. The process according to claim 1, wherein a nonhuman organism is used which, as the wild-type, already has a ketolase activity, and the genetic modification causes an increase in the ketolase activity in comparison with the wild-type.

3. The process according to claim 2, wherein for increasing the ketolase activity gene expression of a nucleic acid encoding a ketolase is increased compared to the wild-type.

4. The process according to claim 3, wherein for increasing the gene expression a nucleic acid which encodes a ketolase is inserted into the organism.

5. The process according to claim 4, wherein, as a nucleic acid encoding a ketolase, a nucleic acid is inserted which encodes a ketolase comprising the amino acid sequence SEQ ID NO: 4 or a sequence derived from this sequence by substitution, insertion or deletion of amino acids, which has an identity of at least 70% at the amino acid level with the sequence SEQ ID NO: 4.

6. The process according to claim 1, wherein a nonhuman organism is used which, as the wild-type, has no ketolase activity and the genetic modification causes a ketolase activity in comparison with the wild-type.

7. The process according to claim 6, wherein a genetically modified organism is used which transgenically expresses a ketolase.

8. The process according to claim 6, wherein, for causing gene expression, a nucleic acid which encodes a ketolase is inserted into the organism.

9. The process according to claim 8, wherein a nucleic acid is inserted encoding a ketolase comprising the amino acid sequence of SEQ ID NO: 4 or a sequence derived from this sequence by substitution, insertion or deletion of amino acids, which has an identity of at least 70% at the amino acid level with the sequence of SEQ ID NO: 4.

10-62. (canceled)

63. A genetically modified, nonhuman organism, where the genetic modification,
A for the case where the wild-type organism already has a ketolase activity, increases the activity of a ketolase compared to the wild-type and
B for the case where the wild-type organism has no ketolase activity, causes the activity of a ketolase compared to the wild-type,
and where the genetic modification,
C for the case where the wild-type organism already has a β-cyclase activity, increases the activity of a β-cyclase compared to the wild-type and
D for the case where the wild-type organism has no β-cyclase activity, causes the activity of a β-cyclase compared to the wild-type
and the β-cyclase activity increased according to C or caused according to D is caused by a β-cyclase comprising the amino acid sequence of SEQ ID NO: 2 or a sequence derived from this sequence by substitution, insertion or deletion of amino acids, which has an identity of at least 70% at the amino acid level with the sequence of SEQ ID NO: 2.

64-71. (canceled)

72. The process according to claim 7, wherein, for causing the gene expression, a nucleic acid which encodes a ketolase is inserted into the organism.

73. The process according to claim 72, wherein a nucleic acid is inserted encoding a ketolase comprising the amino acid sequence of SEQ ID NO: 4 or a sequence derived from this sequence by substitution, insertion or deletion of amino acids, which has an identity of at least 70% at the amino acid level with the sequence of SEQ ID NO: 4.
